SEQUENCE LISTING (269 sequences)

SEQ ID NO: 1 - DNA, 19 bp, Artificial Sequence
  Adaptor partner strand
agttgatcctggttataca

SEQ ID NO: 2 - DNA, 31 bp, Artificial Sequence
  Adaptor litigation strand
gtgtataaccaggatcaactcccgtgccagt

SEQ ID NO: 3 - DNA, 33 bp, Artificial Sequence
  Index 1 sequencing primer
gtgaaaaccaggatcaactcccgtgccagtcac

SEQ ID NO: 4 - DNA, 30 bp, Artificial Sequence
  Llbrary-free Forward sequencing primer
gtcatgcaggagttgatcctggttatacac

SEQ ID NO: 5 - DNA, 25 bp, Artificial Sequence
  Post-processing amplification primer
actggcacgggagttgatcctggtt

SEQ ID NO: 6 - DNA, 59 bp, Artificial Sequence
  Library-free forward amplification primer
aatgatacggcgaccaccgagatctacacgtcatgcaggagttgatcctggttatacac

SEQ ID NO: 7 - DNA, 65 bp, Artificial Sequence
  Index N701 reverse primer
caagcagaagacggcatacgagattcgccttagtgactggcacgggagttgatcctggttttcac

SEQ ID NO: 8 - DNA, 63 bp, Artificial Sequence
  Index N702 reverse primer
cagcagaagacggcatacgagatctagtacggtgactggcacgggagttgatcctggtttcac

SEQ ID NO: 9 - DNA, 64 bp, Artificial Sequence
  Index N703 reverse primer
caagcagaagacggcatacgagattctgcctgtgactggcacgggagttgatcctggttttcac

SEQ ID NO: 10 - DNA, 64 bp, Artificial Sequence
  Index N704 reverse primer
cagcagaagacggcatacgagatgctcaggagtgactggcacgggagttgatcctggttttcac

SEQ ID NO: 11 - DNA, 101 bp, Artificial Sequence
  CYP2D6 F
  misc_feature (36)..(41): n is a, c, g, or t
atgtgactggcacgggagttgatcctggttttcacnnnnnnaagcacctagcccccattcctgctgagcaggaggtggcaggtacccagactgggaggtaa

SEQ ID NO: 12 - DNA, 101 bp, Artificial Sequence
  CYP2D6
  misc_feature (36)..(41): n is a, c, g, or t
atgtgactggcacgggagttgatcctggttttcacnnnnnnagtcggtggggccaggatgaggcccagtctgttcacacatggctgctgcctctcagctct

SEQ ID NO: 13 - DNA, 101 bp, Artificial Sequence
  AMY1 F
  misc_feature (36)..(41): n is a, c, g, or t
atgtgactggcacgggagttgatcctggttttcacnnnnnnacctgagtagcatcattgtagttctcgatatctccacttccagttttacatttaccatca

SEQ ID NO: 14 - DNA, 101 bp, Artificial Sequence
  chrX 15 F
  misc_feature (36)..(41): n is a, c, g, or t
atgtgactggcacgggagttgatcctggttttcacnnnnnncctggccctcagccagtacagaaagtcatttgtcaaggccttcagttggcagacgtgctc

SEQ ID NO: 15 - DNA, 101 bp, Artificial Sequence
  chrX 15 R
  misc_feature (36)..(41): n is a, c, g, or t
atgtgactggcacgggagttgatcctggttttcacnnnnnnagaattcattgccagctataaatctgtggaaacgctgccacacaatcttagcacacaaga

SEQ ID NO: 16 - DNA, 101 bp, Artificial Sequence
  chrX 477 F
  misc_feature (36)..(41): n is a, c, g, or t
atgtgactggcacgggagttgatcctggttttcacnnnnnngacttcaaagaaattacaagttgacatcttggactctacccctcgtactttatctcctat

SEQ ID NO: 17 - DNA, 101 bp, Artificial Sequence
  chrX 477 R
  misc_feature (36)..(41): n is a, c, g, or t
atgtgactggcacgggagttgatcctggttttcacnnnnnntctctttggggtcaagaaagaatccctagtggatttgggattctagaggaggtgttataa

SEQ ID NO: 18 - DNA, 101 bp, Artificial Sequence
  chrX 478 F
  misc_feature (36)..(41): n is a, c, g, or t
atgtgactggcacgggagttgatcctggttttcacnnnnnntgcgataccatgctgaagatgagctaacccaaccagccaagcaggcagggctgcgaagga

SEQ ID NO: 19 - DNA, 101 bp, Artificial Sequence
  chrX 478 R
  misc_feature (36)..(41): n is a, c, g, or t
atgtgactggcacgggagttgatcctggttttcacnnnnnnggggtaggtggaaaacccaagtaatgtgattttgtaacatccactgctgcatttgtttgc

SEQ ID NO: 20 - DNA, 101 bp, Artificial Sequence
  chrX 69 F
  misc_feature (36)..(41): n is a, c, g, or t
atgtgactggcacgggagttgatcctggttttcacnnnnnnttacttccctccagttttgttgcttgcaaaacaacagaatcttctctccatgaaatcatg

SEQ ID NO: 21 - DNA, 101 bp, Artificial Sequence
  chrX 69 R
  misc_feature (36)..(41): n is a, c, g, or t
atgtgactggcacgggagttgatcctggttttcacnnnnnncaggggtatctattatccccattttctcacaaaggaaaccaagataaaaggtttaaatgg

SEQ ID NO: 22 - DNA, 101 bp, Artificial Sequence
  PLP1 ex1 F
  misc_feature (36)..(41): n is a, c, g, or t
atgtgactggcacgggagttgatcctggttttcacnnnnnngaaattctcttgtgaattcctgtgtcctcttgaatcttcaatgctaaagtttttgaaact

SEQ ID NO: 23 - DNA, 101 bp, Artificial Sequence
  PLP1 ex2 F
  misc_feature (36)..(41): n is a, c, g, or t
atgtgactggcacgggagttgatcctggttttcacnnnnnngggtttgagtggcatgagctacctactggatgtgcctgactgtttccccttcttcttccc

SEQ ID NO: 24 - DNA, 101 bp, Artificial Sequence
  PLP1 ex2 R
  misc_feature (36)..(41): n is a, c, g, or t
atgtgactggcacgggagttgatcctggttttcacnnnnnnctatctccaggatggagagagggaaaaaaaagatgggtctgtgtgggagggcaggtactt

SEQ ID NO: 25 - DNA, 101 bp, Artificial Sequence
  PLP1 ex3 F
  misc_feature (36)..(41): n is a, c, g, or t
atgtgactggcacgggagttgatcctggttttcacnnnnnngaaagaagccaggtcttcaattaataagattccctggtctcgtttgtctacctgttaatg

SEQ ID NO: 26 - DNA, 101 bp, Artificial Sequence
  PLP1 ex3 M
  misc_feature (36)..(41): n is a, c, g, or t
atgtgactggcacgggagttgatcctggttttcacnnnnnncagactcgcgcccaattttcccccaccccttgttattgccacaaaatcctgaggatgatc

SEQ ID NO: 27 - DNA, 101 bp, Artificial Sequence
  PLP1 ex3 R
  misc_feature (36)..(41): n is a, c, g, or t
atgtgactggcacgggagttgatcctggttttcacnnnnnntctttcttcttcctttatggggccctcctgctggctgagggcttctacaccaccggcgca

SEQ ID NO: 28 - DNA, 101 bp, Artificial Sequence
  PLP1 ex4 F
  misc_feature (36)..(41): n is a, c, g, or t
atgtgactggcacgggagttgatcctggttttcacnnnnnngtttgtgtttctacatctgcaggctgatgctgatttctaaccaccccatgtcaatcattt

SEQ ID NO: 29 - DNA, 101 bp, Artificial Sequence
  PLP1 ex4 R
  misc_feature (36)..(41): n is a, c, g, or t
atgtgactggcacgggagttgatcctggttttcacnnnnnnaaccaaatatatagtgcttccatagtgggtaggagagccaaagcacccgtaccctaactc

SEQ ID NO: 30 - DNA, 101 bp, Artificial Sequence
  PLP1 ex5 F
  misc_feature (36)..(41): n is a, c, g, or t
atgtgactggcacgggagttgatcctggttttcacnnnnnnagtctccatgtggccccgtaactccataaagcttaccctgcttgctttttgtgtcttact

SEQ ID NO: 31 - DNA, 101 bp, Artificial Sequence
  PLP1 ex5 R
  misc_feature (36)..(41): n is a, c, g, or t
atgtgactggcacgggagttgatcctggttttcacnnnnnnccatgggtgtaatttgtatggtattagctactcccttgtaaaataacccaaataacccac

SEQ ID NO: 32 - DNA, 101 bp, Artificial Sequence
  PLP1 ex6 F
  misc_feature (36)..(41): n is a, c, g, or t
atgtgactggcacgggagttgatcctggttttcacnnnnnntttacagtggagcatattactgctgttgcaagaaacagttcttcctctttcattttcctg

SEQ ID NO: 33 - DNA, 101 bp, Artificial Sequence
  PLP1 ex6 R
  misc_feature (36)..(41): n is a, c, g, or t
atgtgactggcacgggagttgatcctggttttcacnnnnnnatagctgtacccacactatctcaggcctatttacttgccaagatcattcaaagtcaactc

SEQ ID NO: 34 - DNA, 101 bp, Artificial Sequence
  PLP1 ex7 F
  misc_feature (36)..(41): n is a, c, g, or t
atgtgactggcacgggagttgatcctggttttcacnnnnnngatttgaggagggagtgctttcttttctactctcattcacattctctcttctgttcccta

SEQ ID NO: 35 - DNA, 101 bp, Artificial Sequence
  PLP1 ex7 R
  misc_feature (36)..(41): n is a, c, g, or t
atgtgactggcacgggagttgatcctggttttcacnnnnnncagcattgtaggctgtgtggttagagcctcgctattagagaaagggggatttctacgggg

SEQ ID NO: 36 - DNA, 101 bp, Artificial Sequence
  KRAS ex1 F
  misc_feature (36)..(41): n is a, c, g, or t
atgtgactggcacgggagttgatcctggttttcacnnnnnntgttacctttaaaagacatctgctttctgccaaaattaatgtgctgaacttaaacttacc

SEQ ID NO: 37 - DNA, 101 bp, Artificial Sequence
  KRAS ex1 R
  misc_feature (36)..(41): n is a, c, g, or t
atgtgactggcacgggagttgatcctggttttcacnnnnnnttcccagtaaattactcttaccaatgcaacagactttaaagaagttgtgttttacaatgc

SEQ ID NO: 38 - DNA, 101 bp, Artificial Sequence
  KRAS ex2 F
  misc_feature (36)..(41): n is a, c, g, or t
atgtgactggcacgggagttgatcctggttttcacnnnnnntaaatgacataacagttatgattttgcagaaaacagatctgtatttatttcagtgttact

SEQ ID NO: 39 - DNA, 101 bp, Artificial Sequence
  KRAS ex2 R
  misc_feature (36)..(41): n is a, c, g, or t
atgtgactggcacgggagttgatcctggttttcacnnnnnngacaggttttgaaagatatttgtgttactaatgactgtgctataacttttttttctttcc

SEQ ID NO: 40 - DNA, 101 bp, Artificial Sequence
  KRAS ex3 F
  misc_feature (36)..(41): n is a, c, g, or t
atgtgactggcacgggagttgatcctggttttcacnnnnnnactcaaaaaataaaaactataattactccttaatgtcagcttattatattcaatttaaac

SEQ ID NO: 41 - DNA, 101 bp, Artificial Sequence
  KRAS ex3 R
  misc_feature (36)..(41): n is a, c, g, or t
atgtgactggcacgggagttgatcctggttttcacnnnnnnaacaccttttttgaagtaaaaggtgcactgtaataatccagactgtgtttctcccttctc

SEQ ID NO: 42 - DNA, 101 bp, Artificial Sequence
  KRAS ex4 F
  misc_feature (36)..(41): n is a, c, g, or t
atgtgactggcacgggagttgatcctggttttcacnnnnnngaaacctttatctgtatcaaagaatggtcctgcaccagtaatatgcatattaaaacaaga

SEQ ID NO: 43 - DNA, 101 bp, Artificial Sequence
  KRAS ex4 R
  misc_feature (36)..(41): n is a, c, g, or t
atgtgactggcacgggagttgatcctggttttcacnnnnnngtgtattaaccttatgtgtgacatgttctaatatagtcacattttcattatttttattat

SEQ ID NO: 44 - DNA, 101 bp, Artificial Sequence
  MYC r1 F1
  misc_feature (36)..(41): n is a, c, g, or t
atgtgactggcacgggagttgatcctggttttcacnnnnnnccccagccagcggtccgcaacccttgccgcatccacgaaactttgcccatagcagcgggc

SEQ ID NO: 45 - DNA, 101 bp, Artificial Sequence
  MYC r1 R1
  misc_feature (36)..(41): n is a, c, g, or t
atgtgactggcacgggagttgatcctggttttcacnnnnnncgactcatctcagcattaaagtgataaaaaaataaattaaaaggcaagtggacttcggtg

SEQ ID NO: 46 - DNA, 101 bp, Artificial Sequence
  MYC r2 F1
  misc_feature (36)..(41): n is a, c, g, or t
atgtgactggcacgggagttgatcctggttttcacnnnnnnctgtggcgcgcactgcgcgctgcgccaggtttccgcaccaagacccctttaactcaagac

SEQ ID NO: 47 - DNA, 101 bp, Artificial Sequence
  MYC r2 F2
  misc_feature (36)..(41): n is a, c, g, or t
atgtgactggcacgggagttgatcctggttttcacnnnnnnttctactgcgacgaggaggagaacttctaccagcagcagcagcagagcgagctgcagccc

SEQ ID NO: 48 - DNA, 101 bp, Artificial Sequence
  MYC r2 F3
  misc_feature (36)..(41): n is a, c, g, or t
atgtgactggcacgggagttgatcctggttttcacnnnnnnaccgagctgctgggaggagacatggtgaaccagagtttcatctgcgacccggacgacgag

SEQ ID NO: 49 - DNA, 101 bp, Artificial Sequence
  MYC r2 F4
  misc_feature (36)..(41): n is a, c, g, or t
atgtgactggcacgggagttgatcctggttttcacnnnnnngccgccgcctcagagtgcatcgacccctcggtggtcttcccctaccctctcaacgacagc

SEQ ID NO: 50 - DNA, 101 bp, Artificial Sequence
  MYC r2 R1
  misc_feature (36)..(41): n is a, c, g, or t
atgtgactggcacgggagttgatcctggttttcacnnnnnnggcggctaggggacaggggcggggtgggcagcagctcgaatttcttccagatatcctcgc

SEQ ID NO: 51 - DNA, 101 bp, Artificial Sequence
  MYC r2 R2
  misc_feature (36)..(41): n is a, c, g, or t
atgtgactggcacgggagttgatcctggttttcacnnnnnnagacgagcttggcggcggccgagaagccgctccacatacagtcctggatgatgatgtttt

SEQ ID NO: 52 - DNA, 101 bp, Artificial Sequence
  MYC r2 R3
  misc_feature (36)..(41): n is a, c, g, or t
atgtgactggcacgggagttgatcctggttttcacnnnnnnaggagagcagagaatccgaggacggagagaaggcgctggagtcttgcgaggcgcaggact

SEQ ID NO: 53 - DNA, 101 bp, Artificial Sequence
  MYC r2 R4
  misc_feature (36)..(41): n is a, c, g, or t
atgtgactggcacgggagttgatcctggttttcacnnnnnntaagagtggcccgttaaataagctgccaatgaaaatgggaaaggtatccagccgcccact

SEQ ID NO: 54 - DNA, 101 bp, Artificial Sequence
  MYC r3 F1
  misc_feature (36)..(41): n is a, c, g, or t
atgtgactggcacgggagttgatcctggttttcacnnnnnnttgtatttgtacagcattaatctggtaattgattattttaatgtaaccttgctaaaggag

SEQ ID NO: 55 - DNA, 101 bp, Artificial Sequence
  MYC r3 F2
  misc_feature (36)..(41): n is a, c, g, or t
atgtgactggcacgggagttgatcctggttttcacnnnnnngaggccacagcaaacctcctcacagcccactggtcctcaagaggtgccacgtctccacac

SEQ ID NO: 56 - DNA, 101 bp, Artificial Sequence
  MYC r3 F3
  misc_feature (36)..(41): n is a, c, g, or t
atgtgactggcacgggagttgatcctggttttcacnnnnnnagaggaggaacgagctaaaacggagcttttttgccctgcgtgaccagatcccggagttgg

SEQ ID NO: 57 - DNA, 101 bp, Artificial Sequence
  MYC r3 R1
  misc_feature (36)..(41): n is a, c, g, or t
atgtgactggcacgggagttgatcctggttttcacnnnnnntccaacttgaccctcttggcagcaggatagtccttccgagtggagggaggcgctgcgtag

SEQ ID NO: 58 - DNA, 101 bp, Artificial Sequence
  MYC r3 R2
  misc_feature (36)..(41): n is a, c, g, or t
atgtgactggcacgggagttgatcctggttttcacnnnnnngcttggacggacaggatgtatgctgtggcttttttaaggataactaccttgggggccttt

SEQ ID NO: 59 - DNA, 101 bp, Artificial Sequence
  MYC r3 R3
  misc_feature (36)..(41): n is a, c, g, or t
atgtgactggcacgggagttgatcctggttttcacnnnnnngcatttgatcatgcatttgaaacaagttcataggtgattgctcaggacatttctgttaga

SEQ ID NO: 60 - DNA, 151 bp, Artificial Sequence
  READ1
acttcaactgtcgaaccctctgtgcattggagtgatgctgctgagtacttctgtgctgtgggtgcgttttcaggaggaggtgctgacggactcacctttggcaaagggactcatctaatcatccagccctgtaagtgcccggtagtgtaga

SEQ ID NO: 61 - DNA, 24 bp, Artificial Sequence
  READ2
gggcacttacagggctggatgatt

SEQ ID NO: 62 - DNA, 89 bp, Artificial Sequence
  TRAJ2_01
  misc_feature (46)..(49): n is a, c, g, or t
cgatgacgatgaccagtccctatagccgcttaagtctacactaccnnnnactcaccagatataatgaatacatgggtccctttcccaaa

SEQ ID NO: 63 - DNA, 89 bp, Artificial Sequence
  TRAJ3_01
  misc_feature (46)..(49): n is a, c, g, or t
cgatgacgatgaccagtccctatagccgcttaagtctacactaccnnnnacttacttggccggatgctgagtctggtccctgatccaaa

SEQ ID NO: 64 - DNA, 89 bp, Artificial Sequence
  TRAJ4_01
  misc_feature (46)..(49): n is a, c, g, or t
cgatgacgatgaccagtccctatagccgcttaagtctacactaccnnnnactcacatgggtgtacagccagcctggtccctgctccaaa

SEQ ID NO: 65 - DNA, 89 bp, Artificial Sequence
  TRAJ5_01
  misc_feature (46)..(49): n is a, c, g, or t
cgatgacgatgaccagtccctatagccgcttaagtctacactaccnnnnacttacttggttgcacttggagtcttgttccactcccaaa

SEQ ID NO: 66 - DNA, 89 bp, Artificial Sequence
  TRAJ6_01
  misc_feature (46)..(49): n is a, c, g, or t
cgatgacgatgaccagtccctatagccgcttaagtctacactaccnnnnacttacacggatgaacaataaggctggttcctcttccaaa

SEQ ID NO: 67 - DNA, 89 bp, Artificial Sequence
  TRAJ7_01
  misc_feature (46)..(49): n is a, c, g, or t
cgatgacgatgaccagtccctatagccgcttaagtctacactaccnnnnacttacttggtatgaccaccacttggttccccttcccaaa

SEQ ID NO: 68 - DNA, 89 bp, Artificial Sequence
  TRAJ8_01
  misc_feature (46)..(49): n is a, c, g, or t
cgatgacgatgaccagtccctatagccgcttaagtctacactaccnnnnacttacttggactgaccagaagtcaggtgccagttccaaa

SEQ ID NO: 69 - DNA, 89 bp, Artificial Sequence
  TRAJ9_01
  misc_feature (46)..(49): n is a, c, g, or t
cgatgacgatgaccagtccctatagccgcttaagtctacactaccnnnnacttacttgctttaacaaatagtcttgttcctgctccaaa

SEQ ID NO: 70 - DNA, 89 bp, Artificial Sequence
  TRAJ10_01
  misc_feature (46)..(49): n is a, c, g, or t
cgatgacgatgaccagtccctatagccgcttaagtctacactaccnnnnacttactgagttccacttttagctgagtgcctgtcccaaa

SEQ ID NO: 71 - DNA, 89 bp, Artificial Sequence
  TRAJ11_01
  misc_feature (46)..(49): n is a, c, g, or t
cgatgacgatgaccagtccctatagccgcttaagtctacactaccnnnnatgtacctggagagactagaagcatagtccccttcccaaa

SEQ ID NO: 72 - DNA, 89 bp, Artificial Sequence
  TRAJ12_01
  misc_feature (46)..(49): n is a, c, g, or t
cgatgacgatgaccagtccctatagccgcttaagtctacactaccnnnnacttaccaggcctgaccagcagtctggtcccactcccgaa

SEQ ID NO: 73 - DNA, 89 bp, Artificial Sequence
  TRAJ13_01
  misc_feature (46)..(49): n is a, c, g, or t
cgatgacgatgaccagtccctatagccgcttaagtctacactaccnnnnactcacttgggatgacttggagctttgttccaattccaaa

SEQ ID NO: 74 - DNA, 89 bp, Artificial Sequence
  TRAJ13_02
  misc_feature (46)..(49): n is a, c, g, or t
cgatgacgatgaccagtccctatagccgcttaagtctacactaccnnnnactcacttgggatgacttggagctttgttccagttccaaa

SEQ ID NO: 75 - DNA, 89 bp, Artificial Sequence
  TRAJ14_01
  misc_feature (46)..(49): n is a, c, g, or t
cgatgacgatgaccagtccctatagccgcttaagtctacactaccnnnnacttaccaggttttactgataatcttgtcccactcccaaa

SEQ ID NO: 76 - DNA, 89 bp, Artificial Sequence
  TRAJ15_01
  misc_feature (46)..(49): n is a, c, g, or t
cgatgacgatgaccagtccctatagccgcttaagtctacactaccnnnnacttactggaactcactgataaggtggttcccttcccaaa

SEQ ID NO: 77 - DNA, 89 bp, Artificial Sequence
  TRAJ15_02
  misc_feature (46)..(49): n is a, c, g, or t
cgatgacgatgaccagtccctatagccgcttaagtctacactaccnnnnacttactggaactcactgataggtgggttcccttcccaaa

SEQ ID NO: 78 - DNA, 89 bp, Artificial Sequence
  TRAJ16_01
  misc_feature (46)..(49): n is a, c, g, or t
cgatgacgatgaccagtccctatagccgcttaagtctacactaccnnnnacttactaagatccacctttaacatggtcccccttgcaaa

SEQ ID NO: 79 - DNA, 89 bp, Artificial Sequence
  TRAJ17_01
  misc_feature (46)..(49): n is a, c, g, or t
cgatgacgatgaccagtccctatagccgcttaagtctacactaccnnnnactcacttggtttaactagcaccctggttcctcctccaaa

SEQ ID NO: 80 - DNA, 89 bp, Artificial Sequence
  TRAJ18_01
  misc_feature (46)..(49): n is a, c, g, or t
cgatgacgatgaccagtccctatagccgcttaagtctacactaccnnnnactcaccaggccagacagtcaactgagttcctcttccaaa

SEQ ID NO: 81 - DNA, 89 bp, Artificial Sequence
  TRAJ20_01
  misc_feature (46)..(49): n is a, c, g, or t
cgatgacgatgaccagtccctatagccgcttaagtctacactaccnnnnacttacttgctcttacagttactgtggttccggctccaaa

SEQ ID NO: 82 - DNA, 89 bp, Artificial Sequence
  TRAJ21_01
  misc_feature (46)..(49): n is a, c, g, or t
cgatgacgatgaccagtccctatagccgcttaagtctacactaccnnnnacttacttggttttacattgagtttggtcccagatccaaa

SEQ ID NO: 83 - DNA, 89 bp, Artificial Sequence
  TRAJ22_01
  misc_feature (46)..(49): n is a, c, g, or t
cgatgacgatgaccagtccctatagccgcttaagtctacactaccnnnnccagatccaaaggtcagttgccttgcagaaccagaagaaa

SEQ ID NO: 84 - DNA, 89 bp, Artificial Sequence
  TRAJ23_01
  misc_feature (46)..(49): n is a, c, g, or t
cgatgacgatgaccagtccctatagccgcttaagtctacactaccnnnnacttactgggtttcacagataactccgttccctgtccgaa

SEQ ID NO: 85 - DNA, 89 bp, Artificial Sequence
  TRAJ23_02
  misc_feature (46)..(49): n is a, c, g, or t
cgatgacgatgaccagtccctatagccgcttaagtctacactaccnnnnacttactgggtttcacagatagctccgttccctgtccgaa

SEQ ID NO: 86 - DNA, 89 bp, Artificial Sequence
  TRAJ24_01
  misc_feature (46)..(49): n is a, c, g, or t
cgatgacgatgaccagtccctatagccgcttaagtctacactaccnnnngcttacctggggtgaccacaacctgggtccctgctccaaa

SEQ ID NO: 87 - DNA, 89 bp, Artificial Sequence
  TRAJ26_01
  misc_feature (46)..(49): n is a, c, g, or t
cgatgacgatgaccagtccctatagccgcttaagtctacactaccnnnnacttacagggcagcacggacaatctggttccgggaccaaa

SEQ ID NO: 88 - DNA, 89 bp, Artificial Sequence
  TRAJ27_01
  misc_feature (46)..(49): n is a, c, g, or t
cgatgacgatgaccagtccctatagccgcttaagtctacactaccnnnnacttacttggcttcacagtgagcgtagtcccatccccaaa

SEQ ID NO: 89 - DNA, 89 bp, Artificial Sequence
  TRAJ28_01
  misc_feature (46)..(49): n is a, c, g, or t
cgatgacgatgaccagtccctatagccgcttaagtctacactaccnnnnacttacttggtatgaccgagagtttggtccccttcccgaa

SEQ ID NO: 90 - DNA, 89 bp, Artificial Sequence
  TRAJ29_01
  misc_feature (46)..(49): n is a, c, g, or t
cgatgacgatgaccagtccctatagccgcttaagtctacactaccnnnnacttacttgcaatcacagaaagtcttgtgccctttccaaa

SEQ ID NO: 91 - DNA, 89 bp, Artificial Sequence
  TRAJ30_01
  misc_feature (46)..(49): n is a, c, g, or t
cgatgacgatgaccagtccctatagccgcttaagtctacactaccnnnnacttactggggagaatatgaagtcgtgtcccttttccaaa

SEQ ID NO: 92 - DNA, 89 bp, Artificial Sequence
  TRAJ31_01
  misc_feature (46)..(49): n is a, c, g, or t
cgatgacgatgaccagtccctatagccgcttaagtctacactaccnnnnacttactgggcttcaccaccagctgagttccatctccaaa

SEQ ID NO: 93 - DNA, 89 bp, Artificial Sequence
  TRAJ32_01
  misc_feature (46)..(49): n is a, c, g, or t
cgatgacgatgaccagtccctatagccgcttaagtctacactaccnnnnacgtacttggctggacagcaagcagagtgccagttccaaa

SEQ ID NO: 94 - DNA, 89 bp, Artificial Sequence
  TRAJ33_01
  misc_feature (46)..(49): n is a, c, g, or t
cgatgacgatgaccagtccctatagccgcttaagtctacactaccnnnnacttacctggctttataattagcttggtcccagcgcccca

SEQ ID NO: 95 - DNA, 89 bp, Artificial Sequence
  TRAJ34_01
  misc_feature (46)..(49): n is a, c, g, or t
cgatgacgatgaccagtccctatagccgcttaagtctacactaccnnnnacttacttggaaagacttgtaatctggtcccagtcccaaa

SEQ ID NO: 96 - DNA, 89 bp, Artificial Sequence
  TRAJ36_01
  misc_feature (46)..(49): n is a, c, g, or t
cgatgacgatgaccagtccctatagccgcttaagtctacactaccnnnnacttacagggaataacggtgagtctcgttccagtcccaaa

SEQ ID NO: 97 - DNA, 89 bp, Artificial Sequence
  TRAJ37_01
  misc_feature (46)..(49): n is a, c, g, or t
cgatgacgatgaccagtccctatagccgcttaagtctacactaccnnnnacctacctggttttacttgtaaagttgtcccttgcccaaa

SEQ ID NO: 98 - DNA, 89 bp, Artificial Sequence
  TRAJ38_01
  misc_feature (46)..(49): n is a, c, g, or t
cgatgacgatgaccagtccctatagccgcttaagtctacactaccnnnnactcactcggatttactgccaggcttgttcccaatcccca

SEQ ID NO: 99 - DNA, 89 bp, Artificial Sequence
  TRAJ39_01
  misc_feature (46)..(49): n is a, c, g, or t
cgatgacgatgaccagtccctatagccgcttaagtctacactaccnnnnactcacggggtttgaccattaaccttgttccccctccaaa

SEQ ID NO: 100 - DNA, 89 bp, Artificial Sequence
  TRAJ40_01
  misc_feature (46)..(49): n is a, c, g, or t
cgatgacgatgaccagtccctatagccgcttaagtctacactaccnnnnactcacttgctaaaaccttcagcctggtgcctgttccaaa

SEQ ID NO: 101 - DNA, 89 bp, Artificial Sequence
  TRAJ41_01
  misc_feature (46)..(49): n is a, c, g, or t
cgatgacgatgaccagtccctatagccgcttaagtctacactaccnnnnactcacggggtgtgaccaacagcgaggtgcctttgccgaa

SEQ ID NO: 102 - DNA, 89 bp, Artificial Sequence
  TRAJ42_01
  misc_feature (46)..(49): n is a, c, g, or t
cgatgacgatgaccagtccctatagccgcttaagtctacactaccnnnnacttacttggtttaacagagagtttagtgccttttccaaa

SEQ ID NO: 103 - DNA, 89 bp, Artificial Sequence
  TRAJ43_01
  misc_feature (46)..(49): n is a, c, g, or t
cgatgacgatgaccagtccctatagccgcttaagtctacactaccnnnnacttacttggttttactgtcagtctggtccctgctccaaa

SEQ ID NO: 104 - DNA, 89 bp, Artificial Sequence
  TRAJ44_01
  misc_feature (46)..(49): n is a, c, g, or t
cgatgacgatgaccagtccctatagccgcttaagtctacactaccnnnnacctaccgagcgtgacctgaagtcttgttccagtcccaaa

SEQ ID NO: 105 - DNA, 89 bp, Artificial Sequence
  TRAJ45_01
  misc_feature (46)..(49): n is a, c, g, or t
cgatgacgatgaccagtccctatagccgcttaagtctacactaccnnnnacttacagggctggatgattagatgagtccctttgccaaa

SEQ ID NO: 106 - DNA, 89 bp, Artificial Sequence
  TRAJ46_01
  misc_feature (46)..(49): n is a, c, g, or t
cgatgacgatgaccagtccctatagccgcttaagtctacactaccnnnnacttactgggcctaactgctaaacgagtcccggtcccaaa

SEQ ID NO: 107 - DNA, 89 bp, Artificial Sequence
  TRAJ47_01
  misc_feature (46)..(49): n is a, c, g, or t
cgatgacgatgaccagtccctatagccgcttaagtctacactaccnnnnactcacaggacttgactctcagaatggttcctgcgccaaa

SEQ ID NO: 108 - DNA, 89 bp, Artificial Sequence
  TRAJ48_01
  misc_feature (46)..(49): n is a, c, g, or t
cgatgacgatgaccagtccctatagccgcttaagtctacactaccnnnnacttactgggtatgatggtgagtcttgttccagtcccaaa

SEQ ID NO: 109 - DNA, 89 bp, Artificial Sequence
  TRAJ49_01
  misc_feature (46)..(49): n is a, c, g, or t
cgatgacgatgaccagtccctatagccgcttaagtctacactaccnnnnacttacttggaatgaccgtcaaacttgtccctgtcccaaa

SEQ ID NO: 110 - DNA, 89 bp, Artificial Sequence
  TRAJ50_01
  misc_feature (46)..(49): n is a, c, g, or t
cgatgacgatgaccagtccctatagccgcttaagtctacactaccnnnnacttacttggaatgactgataagcttgtccctggcccaaa

SEQ ID NO: 111 - DNA, 89 bp, Artificial Sequence
  TRAJ52_01
  misc_feature (46)..(49): n is a, c, g, or t
cgatgacgatgaccagtccctatagccgcttaagtctacactaccnnnnacttacttggatggacagtcaagatggtcccttgtccaaa

SEQ ID NO: 112 - DNA, 89 bp, Artificial Sequence
  TRAJ53_01
  misc_feature (46)..(49): n is a, c, g, or t
cgatgacgatgaccagtccctatagccgcttaagtctacactaccnnnnacttacttggattcacggttaagagagttccttttccaaa

SEQ ID NO: 113 - DNA, 89 bp, Artificial Sequence
  TRAJ54_01
  misc_feature (46)..(49): n is a, c, g, or t
cgatgacgatgaccagtccctatagccgcttaagtctacactaccnnnnacttacttgggttgatagtcagcctggttccttggccaaa

SEQ ID NO: 114 - DNA, 89 bp, Artificial Sequence
  TRAJ56_01
  misc_feature (46)..(49): n is a, c, g, or t
cgatgacgatgaccagtccctatagccgcttaagtctacactaccnnnnacatacctggtctaacactcagagttattccttttccaaa

SEQ ID NO: 115 - DNA, 89 bp, Artificial Sequence
  TRAJ57_01
  misc_feature (46)..(49): n is a, c, g, or t
cgatgacgatgaccagtccctatagccgcttaagtctacactaccnnnnacttacatgggtttactgtcagtttcgttccctttccaaa

SEQ ID NO: 116 - DNA, 89 bp, Artificial Sequence
  TRBJ1-1_V2
  misc_feature (46)..(49): n is a, c, g, or t
cgatgacgatgaccagtccctatagccgcttaagtctacactaccnnnnatgtcttacctacaactgtgagtctggtgccttgtccaaa

SEQ ID NO: 117 - DNA, 89 bp, Artificial Sequence
  TRBJ1-2_V2
  misc_feature (46)..(49): n is a, c, g, or t
cgatgacgatgaccagtccctatagccgcttaagtctacactaccnnnncagccttacctacaacggttaacctggtccccgaaccgaa

SEQ ID NO: 118 - DNA, 89 bp, Artificial Sequence
  TRBJ1-3_V2
  misc_feature (46)..(49): n is a, c, g, or t
cgatgacgatgaccagtccctatagccgcttaagtctacactaccnnnncttactcacctacaacagtgagccaacttccctctccaaa

SEQ ID NO: 119 - DNA, 89 bp, Artificial Sequence
  TRBJ1-4_V2
  misc_feature (46)..(49): n is a, c, g, or t
cgatgacgatgaccagtccctatagccgcttaagtctacactaccnnnntttacatacccaagacagagagctgggttccactgccaaa

SEQ ID NO: 120 - DNA, 89 bp, Artificial Sequence
  TRBJ1-5_V2
  misc_feature (46)..(49): n is a, c, g, or t
cgatgacgatgaccagtccctatagccgcttaagtctacactaccnnnngcaacttacctaggatggagagtcgagtcccatcaccaaa

SEQ ID NO: 121 - DNA, 89 bp, Artificial Sequence
  TRBJ1-6_V2
  misc_feature (46)..(49): n is a, c, g, or t
cgatgacgatgaccagtccctatagccgcttaagtctacactaccnnnncccccatacctgtcacagtgagcctggtcccgttcccaaa

SEQ ID NO: 122 - DNA, 89 bp, Artificial Sequence
  TRBJ2-1_V2
  misc_feature (46)..(49): n is a, c, g, or t
cgatgacgatgaccagtccctatagccgcttaagtctacactaccnnnnccttcttacctagcacggtgagccgtgtccctggcccgaa

SEQ ID NO: 123 - DNA, 89 bp, Artificial Sequence
  TRBJ2-2_V2
  misc_feature (46)..(49): n is a, c, g, or t
cgatgacgatgaccagtccctatagccgcttaagtctacactaccnnnncctccttacccagtacggtcagcctagagccttctccaaa

SEQ ID NO: 124 - DNA, 89 bp, Artificial Sequence
  TRBJ2-3_V2
  misc_feature (46)..(49): n is a, c, g, or t
cgatgacgatgaccagtccctatagccgcttaagtctacactaccnnnncccgcttaccgagcactgtcagccgggtgcctgggccaaa

SEQ ID NO: 125 - DNA, 89 bp, Artificial Sequence
  TRBJ2-4_V2
  misc_feature (46)..(49): n is a, c, g, or t
cgatgacgatgaccagtccctatagccgcttaagtctacactaccnnnnccagcttacccagcactgagagccgggtcccggcgccgaa

SEQ ID NO: 126 - DNA, 89 bp, Artificial Sequence
  TRBJ2-5_V2
  misc_feature (46)..(49): n is a, c, g, or t
cgatgacgatgaccagtccctatagccgcttaagtctacactaccnnnncgcgctcaccgagcaccaggagccgcgtgcctggcccgaa

SEQ ID NO: 127 - DNA, 89 bp, Artificial Sequence
  TRBJ2-6_V2
  misc_feature (46)..(49): n is a, c, g, or t
cgatgacgatgaccagtccctatagccgcttaagtctacactaccnnnnaaaactcacccagcacggtcagcctgctgccggccccgaa

SEQ ID NO: 128 - DNA, 89 bp, Artificial Sequence
  TRBJ2-7_V2
  misc_feature (46)..(49): n is a, c, g, or t
cgatgacgatgaccagtccctatagccgcttaagtctacactaccnnnngaatctcacctgtgaccgtgagcctggtgcccggcccgaa

SEQ ID NO: 129 - DNA, 101 bp, Artificial Sequence
  TRAV1-1
  misc_feature (48)..(51): n is a, c, g, or t
agctcatctgagatgtgactggcacgggagttgatcctggttttcacnnnnacgtctagacacaggagctccagatgaaagactctgcctcttacttctgc

SEQ ID NO: 130 - DNA, 101 bp, Artificial Sequence
  TRAV1-2
  misc_feature (48)..(51): n is a, c, g, or t
agctcatctgagatgtgactggcacgggagttgatcctggttttcacnnnnctacgcgattgaaggagctccagatgaaagactctgcctcttacctctgt

SEQ ID NO: 131 - DNA, 101 bp, Artificial Sequence
  TRAV2
  misc_feature (48)..(51): n is a, c, g, or t
agctcatctgagatgtgactggcacgggagttgatcctggttttcacnnnngacatatcggcctccaggtgcgggaggcagatgctgctgtttactactgt

SEQ ID NO: 132 - DNA, 101 bp, Artificial Sequence
  TRAV3
  misc_feature (48)..(51): n is a, c, g, or t
agctcatctgagatgtgactggcacgggagttgatcctggttttcacnnnntgtgagctcaaccatctgcccttgtgagcgactccgctttgtacttctgt

SEQ ID NO: 133 - DNA, 101 bp, Artificial Sequence
  TRAV4
  misc_feature (48)..(51): n is a, c, g, or t
agctcatctgagatgtgactggcacgggagttgatcctggttttcacnnnnagattacggcgccccgggtttccctgagcgacactgctgtgtactactgc

SEQ ID NO: 134 - DNA, 101 bp, Artificial Sequence
  TRAV5
  misc_feature (48)..(51): n is a, c, g, or t
agctcatctgagatgtgactggcacgggagttgatcctggttttcacnnnncatcctgaagtgcagacacccagactggggactcagctatctacttctgt

SEQ ID NO: 135 - DNA, 101 bp, Artificial Sequence
  TRAV6
  misc_feature (48)..(51): n is a, c, g, or t
agctcatctgagatgtgactggcacgggagttgatcctggttttcacnnnngtgaagtcctcacagcctcccagcctgcagactcagctacctacctctgt

SEQ ID NO: 136 - DNA, 101 bp, Artificial Sequence
  TRAV7
  misc_feature (48)..(51): n is a, c, g, or t
agctcatctgagatgtgactggcacgggagttgatcctggttttcacnnnntccggcattatacagccgtgcagcctgaagattcagccacctatttctgt

SEQ ID NO: 137 - DNA, 101 bp, Artificial Sequence
  TRAV8-1
  misc_feature (48)..(51): n is a, c, g, or t
agctcatctgagatgtgactggcacgggagttgatcctggttttcacnnnnaccgatagctaccctctgtgcagtggagtgacacagctgagtacttctgt

SEQ ID NO: 138 - DNA, 101 bp, Artificial Sequence
  TRAV8-2
  misc_feature (48)..(51): n is a, c, g, or t
agctcatctgagatgtgactggcacgggagttgatcctggttttcacnnnngttagcgatcaccctcagcccatatgagcgacgcggctgagtacttctgt

SEQ ID NO: 139 - DNA, 101 bp, Artificial Sequence
  TRAV8-3
  misc_feature (48)..(51): n is a, c, g, or t
agctcatctgagatgtgactggcacgggagttgatcctggttttcacnnnncaactgtcgaaccctctgtgcattggagtgatgctgctgagtacttctgt

SEQ ID NO: 140 - DNA, 101 bp, Artificial Sequence
  TRAV8-6
  misc_feature (48)..(51): n is a, c, g, or t
agctcatctgagatgtgactggcacgggagttgatcctggttttcacnnnntggtcactagaccctcagtccatataagcgacacggctgagtacttctgt

SEQ ID NO: 141 - DNA, 101 bp, Artificial Sequence
  TRAV9-1
  misc_feature (48)..(51): n is a, c, g, or t
agctcatctgagatgtgactggcacgggagttgatcctggttttcacnnnnagcgatgtcaagactcagttcaagagtcagactccgctgtgtacttctgt

SEQ ID NO: 142 - DNA, 101 bp, Artificial Sequence
  TRAV9-2
  misc_feature (48)..(51): n is a, c, g, or t
agctcatctgagatgtgactggcacgggagttgatcctggttttcacnnnncttacgactgaggctcagttcaagtgtcagactcagcggtgtacttctgt

SEQ ID NO: 143 - DNA, 101 bp, Artificial Sequence
  TRAV10
  misc_feature (48)..(51): n is a, c, g, or t
agctcatctgagatgtgactggcacgggagttgatcctggttttcacnnnngagctacagtcacagcctcccagctcagcgattcagcctcctacatctgt

SEQ ID NO: 144 - DNA, 101 bp, Artificial Sequence
  TRAV12-1
  misc_feature (48)..(51): n is a, c, g, or t
agctcatctgagatgtgactggcacgggagttgatcctggttttcacnnnntcatgctgaccagagactccaagctcagtgattcagccacctacctctgt

SEQ ID NO: 145 - DNA, 101 bp, Artificial Sequence
  TRAV12-2
  misc_feature (48)..(51): n is a, c, g, or t
agctcatctgagatgtgactggcacgggagttgatcctggttttcacnnnnaccttcgagacagagactcccagcccagtgattcagccacctacctctgt

SEQ ID NO: 146 - DNA, 101 bp, Artificial Sequence
  TRAV12-3
  misc_feature (48)..(51): n is a, c, g, or t
agctcatctgagatgtgactggcacgggagttgatcctggttttcacnnnncttcgtagaccagagactcacagcccagtgattcagccacctacctctgt

SEQ ID NO: 147 - DNA, 101 bp, Artificial Sequence
  TRAV13-1
  misc_feature (48)..(51): n is a, c, g, or t
agctcatctgagatgtgactggcacgggagttgatcctggttttcacnnnngaggaactctcacagagacccaacctgaagactcggctgtctacttctgt

SEQ ID NO: 148 - DNA, 101 bp, Artificial Sequence
  TRAV13-2
  misc_feature (48)..(51): n is a, c, g, or t
agctcatctgagatgtgactggcacgggagttgatcctggttttcacnnnntgaacgtctgtgcagctactcaacctggagactcagctgtctacttttgt

SEQ ID NO: 149 - DNA, 101 bp, Artificial Sequence
  TRAV14/DV4
  misc_feature (48)..(51): n is a, c, g, or t
agctcatctgagatgtgactggcacgggagttgatcctggttttcacnnnnaggactcagtctccgcttcacaactgggggactcagcaatgtatttctgt

SEQ ID NO: 150 - DNA, 101 bp, Artificial Sequence
  TRAV14/DV4
  misc_feature (48)..(51): n is a, c, g, or t
agctcatctgagatgtgactggcacgggagttgatcctggttttcacnnnncaagtgtcacctccgcttcacaactgggggactcagcaatgtatttctgt

SEQ ID NO: 151 - DNA, 101 bp, Artificial Sequence
  TRAV16
  misc_feature (48)..(51): n is a, c, g, or t
agctcatctgagatgtgactggcacgggagttgatcctggttttcacnnnngtctgagtcaaccatttgctcaagaggaagactcagccatgtattactgt

SEQ ID NO: 152 - DNA, 101 bp, Artificial Sequence
  TRAV17
  misc_feature (48)..(51): n is a, c, g, or t
agctcatctgagatgtgactggcacgggagttgatcctggttttcacnnnntctcacagtgcacggcttcccgggcagcagacactgcttcttacttctgt

SEQ ID NO: 153 - DNA, 101 bp, Artificial Sequence
  TRAV18
  misc_feature (48)..(51): n is a, c, g, or t
agctcatctgagatgtgactggcacgggagttgatcctggttttcacnnnnaccaggatctgccctcggtgcagctgtcggactctgccgtgtactactgc

SEQ ID NO: 154 - DNA, 101 bp, Artificial Sequence
  TRAV19
  misc_feature (48)..(51): n is a, c, g, or t
agctcatctgagatgtgactggcacgggagttgatcctggttttcacnnnngttgaacgtccacagcctcacaagtcgtggactcagcagtatacttctgt

SEQ ID NO: 155 - DNA, 101 bp, Artificial Sequence
  TRAV20
  misc_feature (48)..(51): n is a, c, g, or t
agctcatctgagatgtgactggcacgggagttgatcctggttttcacnnnncagtcctagacacagcccctaaacctgaagactcagccacttatctctgt

SEQ ID NO: 156 - DNA, 101 bp, Artificial Sequence
  TRAV21
  misc_feature (48)..(51): n is a, c, g, or t
agctcatctgagatgtgactggcacgggagttgatcctggttttcacnnnntgacttgcagtgcagcttctcagcctggtgactcagccacctacctctgt

SEQ ID NO: 157 - DNA, 101 bp, Artificial Sequence
  TRAV22
  misc_feature (48)..(51): n is a, c, g, or t
agctcatctgagatgtgactggcacgggagttgatcctggttttcacnnnnaggacgacttttcctcttcccagaccacagactcaggcgtttatttctgt

SEQ ID NO: 158 - DNA, 101 bp, Artificial Sequence
  TRAV23/DV6
  misc_feature (48)..(51): n is a, c, g, or t
agctcatctgagatgtgactggcacgggagttgatcctggttttcacnnnnctagtactcgcatggattcccagcctggagactcagccacctacttctgt

SEQ ID NO: 159 - DNA, 101 bp, Artificial Sequence
  TRAV24
  misc_feature (48)..(51): n is a, c, g, or t
agctcatctgagatgtgactggcacgggagttgatcctggttttcacnnnngactgctagacaaaggatcccagcctgaagactcagccacatacctctgt

SEQ ID NO: 160 - DNA, 101 bp, Artificial Sequence
  TRAV25
  misc_feature (48)..(51): n is a, c, g, or t
agctcatctgagatgtgactggcacgggagttgatcctggttttcacnnnntctcatggaccacagccacccagactacagatgtaggaacctacttctgt

SEQ ID NO: 161 - DNA, 101 bp, Artificial Sequence
  TRAV26-1
  misc_feature (48)..(51): n is a, c, g, or t
agctcatctgagatgtgactggcacgggagttgatcctggttttcacnnnnacgttcagcagccccacgctacgctgagagacactgctgtgtactattgc

SEQ ID NO: 162 - DNA, 101 bp, Artificial Sequence
  TRAV26-2
  misc_feature (48)..(51): n is a, c, g, or t
agctcatctgagatgtgactggcacgggagttgatcctggttttcacnnnnctacgttagcgcaccgtgctaccttgagagatgctgctgtgtactactgc

SEQ ID NO: 163 - DNA, 101 bp, Artificial Sequence
  TRAV27
  misc_feature (48)..(51): n is a, c, g, or t
agctcatctgagatgtgactggcacgggagttgatcctggttttcacnnnngacaaggcttcactgcagcccagcctggtgatacaggcctctacctctgt

SEQ ID NO: 164 - DNA, 101 bp, Artificial Sequence
  TRAV29/DV5
  misc_feature (48)..(51): n is a, c, g, or t
agctcatctgagatgtgactggcacgggagttgatcctggttttcacnnnntgtgcactagtgtgccctcccagcctggagactctgcagtgtacttctgt

SEQ ID NO: 165 - DNA, 101 bp, Artificial Sequence
  TRAV30
  misc_feature (48)..(51): n is a, c, g, or t
agctcatctgagatgtgactggcacgggagttgatcctggttttcacnnnnagaatgcctgtacggcctcccagctcagttactcaggaacctacttctgc

SEQ ID NO: 166 - DNA, 101 bp, Artificial Sequence
  TRAV34
  misc_feature (48)..(51): n is a, c, g, or t
agctcatctgagatgtgactggcacgggagttgatcctggttttcacnnnncagtcagtcacacagcctcccagcccagccatgcaggcatctacctctgt

SEQ ID NO: 167 - DNA, 101 bp, Artificial Sequence
  TRAV35
  misc_feature (48)..(51): n is a, c, g, or t
agctcatctgagatgtgactggcacgggagttgatcctggttttcacnnnngttgactagcctcagcatccatacctagtgatgtaggcatctacttctgt

SEQ ID NO: 168 - DNA, 101 bp, Artificial Sequence
  TRAV36/DV7
  misc_feature (48)..(51): n is a, c, g, or t
agctcatctgagatgtgactggcacgggagttgatcctggttttcacnnnntcccgtagatcacagccacccagaccggagactcggccatctacctctgt

SEQ ID NO: 169 - DNA, 101 bp, Artificial Sequence
  TRAV38-1
  misc_feature (48)..(51): n is a, c, g, or t
agctcatctgagatgtgactggcacgggagttgatcctggttttcacnnnnacgctcgtaactcagactcacagctgggggacactgcgatgtatttctgt

SEQ ID NO: 170 - DNA, 101 bp, Artificial Sequence
  TRAV38-2/DV8
  misc_feature (48)..(51): n is a, c, g, or t
agctcatctgagatgtgactggcacgggagttgatcctggttttcacnnnngtatggactcctcagactcacagctgggggatgccgcgatgtatttctgt

SEQ ID NO: 171 - DNA, 101 bp, Artificial Sequence
  TRAV39
  misc_feature (48)..(51): n is a, c, g, or t
agctcatctgagatgtgactggcacgggagttgatcctggttttcacnnnncacgatcagtcacagctgccgtgcatgacctctctgccacctacttctgt

SEQ ID NO: 172 - DNA, 101 bp, Artificial Sequence
  TRAV40
  misc_feature (48)..(51): n is a, c, g, or t
agctcatctgagatgtgactggcacgggagttgatcctggttttcacnnnntgtacatgcgatattcagtccaggtatcagactcagccgtgtactactgt

SEQ ID NO: 173 - DNA, 101 bp, Artificial Sequence
  TRAV41
  misc_feature (48)..(51): n is a, c, g, or t
agctcatctgagatgtgactggcacgggagttgatcctggttttcacnnnnagacgacttgcacagcctcccatcccagagactctgccgtctacatctgt

SEQ ID NO: 174 - DNA, 101 bp, Artificial Sequence
  TRBV2_01
  misc_feature (48)..(51): n is a, c, g, or t
agctcatctgagatgtgactggcacgggagttgatcctggttttcacnnnntcgcctatagtccggtccacaaagctggaggactcagccatgtacttctg

SEQ ID NO: 175 - DNA, 101 bp, Artificial Sequence
  TRBV3-1_01
  misc_feature (48)..(51): n is a, c, g, or t
agctcatctgagatgtgactggcacgggagttgatcctggttttcacnnnngtctgacagttcaattccctggagcttggtgactctgctgtgtatttctg

SEQ ID NO: 176 - DNA, 101 bp, Artificial Sequence
  TRBV4-1_01
  misc_feature (48)..(51): n is a, c, g, or t
agctcatctgagatgtgactggcacgggagttgatcctggttttcacnnnncataagtgcctacacgccctgcagccagaagactcagccctgtatctctg

SEQ ID NO: 177 - DNA, 101 bp, Artificial Sequence
  TRBV4-2_01
  misc_feature (48)..(51): n is a, c, g, or t
agctcatctgagatgtgactggcacgggagttgatcctggttttcacnnnnagagtcgctatacacaccctgcagccagaagactcggccctgtatctctg

SEQ ID NO: 178 - DNA, 101 bp, Artificial Sequence
  TRBV5-1_01
  misc_feature (48)..(51): n is a, c, g, or t
agctcatctgagatgtgactggcacgggagttgatcctggttttcacnnnntcgaactctggtgagcaccttggagctgggggactcggccctttatcttt

SEQ ID NO: 179 - DNA, 101 bp, Artificial Sequence
  TRBV5-4_01
  misc_feature (48)..(51): n is a, c, g, or t
agctcatctgagatgtgactggcacgggagttgatcctggttttcacnnnngtcgtgatacgtgaacgccttggagctggacgactcggccctgtatctct

SEQ ID NO: 180 - DNA, 101 bp, Artificial Sequence
  TRBV5-5_01
  misc_feature (48)..(51): n is a, c, g, or t
agctcatctgagatgtgactggcacgggagttgatcctggttttcacnnnncaacctgagtgtgaacgccttgttgctgggggactcggccctgtatctct

SEQ ID NO: 181 - DNA, 101 bp, Artificial Sequence
  TRBV5-5_01b
  misc_feature (48)..(51): n is a, c, g, or t
agctcatctgagatgtgactggcacgggagttgatcctggttttcacnnnnagttgacgcagtgaacgccttgttgctgggggactcggccctgtatctct

SEQ ID NO: 182 - DNA, 101 bp, Artificial Sequence
  TRBV5-5_01c
  misc_feature (48)..(51): n is a, c, g, or t
agctcatctgagatgtgactggcacgggagttgatcctggttttcacnnnntccctgagtagtgaacgccttgttgctgggggactcggccctgtatctct

SEQ ID NO: 183 - DNA, 101 bp, Artificial Sequence
  TRBV5-5_01d
  misc_feature (48)..(51): n is a, c, g, or t
agctcatctgagatgtgactggcacgggagttgatcctggttttcacnnnngtggactcatgtgaacgccttgttgctgggggactcggccctgtatctct

SEQ ID NO: 184 - DNA, 101 bp, Artificial Sequence
  TRBV5-6_01
  misc_feature (48)..(51): n is a, c, g, or t
agctcatctgagatgtgactggcacgggagttgatcctggttttcacnnnncatagtcagcgtgaacgccttgttgctgggggactcggccctctatctct

SEQ ID NO: 185 - DNA, 101 bp, Artificial Sequence
  TRBV5-8_01
  misc_feature (48)..(51): n is a, c, g, or t
agctcatctgagatgtgactggcacgggagttgatcctggttttcacnnnnagatcagtcggtgaacgccttggagctggaggactcggccctgtatctct

SEQ ID NO: 186 - DNA, 101 bp, Artificial Sequence
  TRBV6-1_01
  misc_feature (48)..(51): n is a, c, g, or t
agctcatctgagatgtgactggcacgggagttgatcctggttttcacnnnntcagcgattctggagtcggctgctccctcccagacatctgtgtacttctg

SEQ ID NO: 187 - DNA, 101 bp, Artificial Sequence
  TRBV6-2_01
  misc_feature (48)..(51): n is a, c, g, or t
agctcatctgagatgtgactggcacgggagttgatcctggttttcacnnnngtcttcgaagtggagtcggctgctccctcccaaacatctgtgtacttctg

SEQ ID NO: 188 - DNA, 101 bp, Artificial Sequence
  TRBV6-4_01
  misc_feature (48)..(51): n is a, c, g, or t
agctcatctgagatgtgactggcacgggagttgatcctggttttcacnnnncatcatcggatggcgtctgctgtaccctctcagacatctgtgtacttctg

SEQ ID NO: 189 - DNA, 101 bp, Artificial Sequence
  TRBV6-5_01
  misc_feature (48)..(51): n is a, c, g, or t
agctcatctgagatgtgactggcacgggagttgatcctggttttcacnnnnaggagatccttgctgtcggctgctccctcccagacatctgtgtacttctg

SEQ ID NO: 190 - DNA, 101 bp, Artificial Sequence
  TRBV6-6_01
  misc_feature (48)..(51): n is a, c, g, or t
agctcatctgagatgtgactggcacgggagttgatcctggttttcacnnnntccttcgaagtggagttggctgctccctcccagacatctgtgtacttctg

SEQ ID NO: 191 - DNA, 101 bp, Artificial Sequence
  TRBV6-8_01
  misc_feature (48)..(51): n is a, c, g, or t
agctcatctgagatgtgactggcacgggagttgatcctggttttcacnnnngtgaagcttctggtgtcggctgctccctcccagacatctgtgtacttgtg

SEQ ID NO: 192 - DNA, 101 bp, Artificial Sequence
  TRBV6-9_01
  misc_feature (48)..(51): n is a, c, g, or t
agctcatctgagatgtgactggcacgggagttgatcctggttttcacnnnncatggtaccatggagtcagctgctccctcccagacatctgtatacttctg

SEQ ID NO: 193 - DNA, 101 bp, Artificial Sequence
  TRBV7-2_01
  misc_feature (48)..(51): n is a, c, g, or t
agctcatctgagatgtgactggcacgggagttgatcctggttttcacnnnnagaccatggttccagcgcacacagcaggaggactcggccgtgtatctctg

SEQ ID NO: 194 - DNA, 101 bp, Artificial Sequence
  TRBV7-3_01
  misc_feature (48)..(51): n is a, c, g, or t
agctcatctgagatgtgactggcacgggagttgatcctggttttcacnnnntcgctgcaattccagcgcacagagcggggggactcagccgtgtatctctg

SEQ ID NO: 195 - DNA, 101 bp, Artificial Sequence
  TRBV7-4_01
  misc_feature (48)..(51): n is a, c, g, or t
agctcatctgagatgtgactggcacgggagttgatcctggttttcacnnnngttgacgctatccagcgcacagagcagggggactcagctgtgtatctctg

SEQ ID NO: 196 - DNA, 101 bp, Artificial Sequence
  TRBV7-6_01
  misc_feature (48)..(51): n is a, c, g, or t
agctcatctgagatgtgactggcacgggagttgatcctggttttcacnnnncacagattcgtccagcgcacagagcagcgggactcggccatgtatcgctg

SEQ ID NO: 197 - DNA, 101 bp, Artificial Sequence
  TRBV7-7_01
  misc_feature (48)..(51): n is a, c, g, or t
agctcatctgagatgtgactggcacgggagttgatcctggttttcacnnnnagatctaggcttcagcgcacagagcagcgggactcagccatgtatcgctg

SEQ ID NO: 198 - DNA, 101 bp, Artificial Sequence
  TRBV7-8_01
  misc_feature (48)..(51): n is a, c, g, or t
agctcatctgagatgtgactggcacgggagttgatcctggttttcacnnnntcgccattagtccagcgcacacagcaggaggactccgccgtgtatctctg

SEQ ID NO: 199 - DNA, 101 bp, Artificial Sequence
  TRBV7-9_01
  misc_feature (48)..(51): n is a, c, g, or t
agctcatctgagatgtgactggcacgggagttgatcctggttttcacnnnngtcgtgaatctccagcgcacagagcagggggactcggccatgtatctctg

SEQ ID NO: 200 - DNA, 101 bp, Artificial Sequence
  TRBV9_01
  misc_feature (48)..(51): n is a, c, g, or t
agctcatctgagatgtgactggcacgggagttgatcctggttttcacnnnncataacggctctgagctctctggagctgggggactcagctttgtatttct

SEQ ID NO: 201 - DNA, 101 bp, Artificial Sequence
  TRBV10-1_01
  misc_feature (48)..(51): n is a, c, g, or t
agctcatctgagatgtgactggcacgggagttgatcctggttttcacnnnnagatgtccgatggagtctgctgcctcctcccagacatctgtatatttctg

SEQ ID NO: 202 - DNA, 101 bp, Artificial Sequence
  TRBV10-2_01
  misc_feature (48)..(51): n is a, c, g, or t
agctcatctgagatgtgactggcacgggagttgatcctggttttcacnnnntcactaggtctggagtcagctacccgctcccagacatctgtgtatttctg

SEQ ID NO: 203 - DNA, 101 bp, Artificial Sequence
  TRBV10-3_01
  misc_feature (48)..(51): n is a, c, g, or t
agctcatctgagatgtgactggcacgggagttgatcctggttttcacnnnngttagtccgatggagtccgctaccagctcccagacatctgtgtacttctg

SEQ ID NO: 204 - DNA, 101 bp, Artificial Sequence
  TRBV11-1_01
  misc_feature (48)..(51): n is a, c, g, or t
agctcatctgagatgtgactggcacgggagttgatcctggttttcacnnnncagtcgaacttccagcctgcagagcttggggactcggccatgtatctctg

SEQ ID NO: 205 - DNA, 101 bp, Artificial Sequence
  TRBV11-2_01
  misc_feature (48)..(51): n is a, c, g, or t
agctcatctgagatgtgactggcacgggagttgatcctggttttcacnnnnagcgacttagtccagcctgcaaagcttgaggactcggccgtgtatctctg

SEQ ID NO: 206 - DNA, 101 bp, Artificial Sequence
  TRBV11-3_01
  misc_feature (48)..(51): n is a, c, g, or t
agctcatctgagatgtgactggcacgggagttgatcctggttttcacnnnntcgcgtcatatccagcctgcagagcttggggactcggccgtgtatctctg

SEQ ID NO: 207 - DNA, 101 bp, Artificial Sequence
  TRBV12-3_01
  misc_feature (48)..(51): n is a, c, g, or t
agctcatctgagatgtgactggcacgggagttgatcctggttttcacnnnngttatgacgctccagccctcagaacccagggactcagctgtgtacttctg

SEQ ID NO: 208 - DNA, 101 bp, Artificial Sequence
  TRBV12-5_01
  misc_feature (48)..(51): n is a, c, g, or t
agctcatctgagatgtgactggcacgggagttgatcctggttttcacnnnncaatactgcgtccagccctcagaacccagggactcagctgtgtatttttg

SEQ ID NO: 209 - DNA, 101 bp, Artificial Sequence
  TRBV13_01
  misc_feature (48)..(51): n is a, c, g, or t
agctcatctgagatgtgactggcacgggagttgatcctggttttcacnnnnagcgcagtattgagctccttggagctgggggactcagccctgtacttctg

SEQ ID NO: 210 - DNA, 101 bp, Artificial Sequence
  TRBV14_01
  misc_feature (48)..(51): n is a, c, g, or t
agctcatctgagatgtgactggcacgggagttgatcctggttttcacnnnntcgtagactctgcagcctgcagaactggaggattctggagtttatttctg

SEQ ID NO: 211 - DNA, 101 bp, Artificial Sequence
  TRBV15_01
  misc_feature (48)..(51): n is a, c, g, or t
agctcatctgagatgtgactggcacgggagttgatcctggttttcacnnnngtagtcctgatccgctcaccaggcctgggggacacagccatgtacctgtg

SEQ ID NO: 212 - DNA, 101 bp, Artificial Sequence
  TRBV16_01
  misc_feature (48)..(51): n is a, c, g, or t
agctcatctgagatgtgactggcacgggagttgatcctggttttcacnnnncatcgagacttccaggctacgaagcttgaggattcagcagtgtatttttg

SEQ ID NO: 213 - DNA, 101 bp, Artificial Sequence
  TRBV18_01
  misc_feature (48)..(51): n is a, c, g, or t
agctcatctgagatgtgactggcacgggagttgatcctggttttcacnnnnagcacttgagtccagcaggtagtgcgaggagattcggcagcttatttctg

SEQ ID NO: 214 - DNA, 101 bp, Artificial Sequence
  TRBV19_01
  misc_feature (48)..(51): n is a, c, g, or t
agctcatctgagatgtgactggcacgggagttgatcctggttttcacnnnntccaagttgctgacatcggcccaaaagaacccgacagctttctatctctg

SEQ ID NO: 215 - DNA, 101 bp, Artificial Sequence
  TRBV20-1_01
  misc_feature (48)..(51): n is a, c, g, or t
agctcatctgagatgtgactggcacgggagttgatcctggttttcacnnnngtactcggtacagtgaccagtgcccatcctgaagacagcagcttctacat

SEQ ID NO: 216 - DNA, 101 bp, Artificial Sequence
  TRBV20-1_01b
  misc_feature (48)..(51): n is a, c, g, or t
agctcatctgagatgtgactggcacgggagttgatcctggttttcacnnnncatggaccatcagtgaccagtgcccatcctgaagacagcagcttctacat

SEQ ID NO: 217 - DNA, 101 bp, Artificial Sequence
  TRBV20-1_01c
  misc_feature (48)..(51): n is a, c, g, or t
agctcatctgagatgtgactggcacgggagttgatcctggttttcacnnnnaggtctaacgcagtgaccagtgcccatcctgaagacagcagcttctacat

SEQ ID NO: 218 - DNA, 101 bp, Artificial Sequence
  TRBV20-1_01d
  misc_feature (48)..(51): n is a, c, g, or t
agctcatctgagatgtgactggcacgggagttgatcctggttttcacnnnntgagatgctccagtgaccagtgcccatcctgaagacagcagcttctacat

SEQ ID NO: 219 - DNA, 101 bp, Artificial Sequence
  TRBV24-1_01
  misc_feature (48)..(51): n is a, c, g, or t
agctcatctgagatgtgactggcacgggagttgatcctggttttcacnnnngatctacgagagagtctgccatccccaaccagacagctctttacttctgt

SEQ ID NO: 220 - DNA, 101 bp, Artificial Sequence
  TRBV25-1_01
  misc_feature (48)..(51): n is a, c, g, or t
agctcatctgagatgtgactggcacgggagttgatcctggttttcacnnnnctctcgtagatggagtctgccaggccctcacatacctctcagtacctctg

SEQ ID NO: 221 - DNA, 101 bp, Artificial Sequence
  TRBV27_01
  misc_feature (48)..(51): n is a, c, g, or t
agctcatctgagatgtgactggcacgggagttgatcctggttttcacnnnnacgagcatcttggagtcgcccagccccaaccagacctctctgtacttctg

SEQ ID NO: 222 - DNA, 101 bp, Artificial Sequence
  TRBV28_01
  misc_feature (48)..(51): n is a, c, g, or t
agctcatctgagatgtgactggcacgggagttgatcctggttttcacnnnntgcttcgaagtggagtccgccagcaccaaccagacatctatgtacctctg

SEQ ID NO: 223 - DNA, 101 bp, Artificial Sequence
  TRBV29-1_01
  misc_feature (48)..(51): n is a, c, g, or t
agctcatctgagatgtgactggcacgggagttgatcctggttttcacnnnngagaagcttcctgtgagcaacatgagccctgaagacagcagcatatatct

SEQ ID NO: 224 - DNA, 101 bp, Artificial Sequence
  TRBV29-1_01b
  misc_feature (48)..(51): n is a, c, g, or t
agctcatctgagatgtgactggcacgggagttgatcctggttttcacnnnncttggtaccactgtgagcaacatgagccctgaagacagcagcatatatct

SEQ ID NO: 225 - DNA, 101 bp, Artificial Sequence
  TRBV29-1_01c
  misc_feature (48)..(51): n is a, c, g, or t
agctcatctgagatgtgactggcacgggagttgatcctggttttcacnnnnacaccatggtctgtgagcaacatgagccctgaagacagcagcatatatct

SEQ ID NO: 226 - DNA, 101 bp, Artificial Sequence
  TRBV29-1_01d
  misc_feature (48)..(51): n is a, c, g, or t
agctcatctgagatgtgactggcacgggagttgatcctggttttcacnnnntgatcacgtgctgtgagcaacatgagccctgaagacagcagcatatatct

SEQ ID NO: 227 - DNA, 101 bp, Artificial Sequence
  TRBV30_01
  misc_feature (48)..(51): n is a, c, g, or t
agctcatctgagatgtgactggcacgggagttgatcctggttttcacnnnngacatggtacgttctaagaagctccttctcagtgactctggcttctatct

SEQ ID NO: 228 - DNA, 27 bp, Artificial Sequence
  ACC4_27
cctatagccgcttaagtctacactacc

SEQ ID NO: 229 - DNA, 62 bp, Artificial Sequence
  CAC3 FLFP
aatgatacggcgaccaccgagatctacacgtgactggcacgggagttgatcctggttttcac

SEQ ID NO: 230 - DNA, 33 bp, Artificial Sequence
  TCR_FSP
gtgactggcacgggagttgatcctggttttcac

SEQ ID NO: 231 - DNA, 35 bp, Artificial Sequence
  TCR-HT_RSP
acacgtcacctatagccgcttaagtctacactacc

SEQ ID NO: 232 - DNA, 45 bp, Artificial Sequence
  J-probe complement
ggtagtgtagacttaagcggctatagggactggtcatcgtcatcg

SEQ ID NO: 233 - DNA, 19 bp, Artificial Sequence
  J-probe-part
ccgcttaagtctacactac

SEQ ID NO: 234 - DNA, 27 bp, Artificial Sequence
  J-probe-lig
ggtagtgtagacttaagcggctatagg

SEQ ID NO: 235 - DNA, 35 bp, Artificial Sequence
  TCR-HT ISP
ggtagtgtagacttaagcggctataggtgacgtgt

SEQ ID NO: 236 - DNA, 67 bp, Artificial Sequence
  TCR-HT ACC4 FLRIP-1
caagcagaagacggcatacgagatacgatgctacacgtcacctatagccgcttaagtctacactacc

SEQ ID NO: 237 - DNA, 67 bp, Artificial Sequence
  TCR-HT ACC4 FLRIP-2
caagcagaagacggcatacgagatagtctgacacacgtcacctatagccgcttaagtctacactacc

SEQ ID NO: 238 - DNA, 67 bp, Artificial Sequence
  TCR-HT ACC4 FLRIP-3
caagcagaagacggcatacgagatccaggattacacgtcacctatagccgcttaagtctacactacc

SEQ ID NO: 239 - DNA, 67 bp, Artificial Sequence
  TCR-HT ACC4 FLRIP-4
caagcagaagacggcatacgagattcggatcaacacgtcacctatagccgcttaagtctacactacc

SEQ ID NO: 240 - DNA, 67 bp, Artificial Sequence
  TCR-HT ACC4 FLRIP-5
caagcagaagacggcatacgagataagccgttacacgtcacctatagccgcttaagtctacactacc

SEQ ID NO: 241 - DNA, 67 bp, Artificial Sequence
  TCR-HT ACC4 FLRIP-6
caagcagaagacggcatacgagatcacgtagtacacgtcacctatagccgcttaagtctacactacc

SEQ ID NO: 242 - DNA, 67 bp, Artificial Sequence
  TCR-HT ACC4 FLRIP-7
caagcagaagacggcatacgagatagtcctagacacgtcacctatagccgcttaagtctacactacc

SEQ ID NO: 243 - DNA, 67 bp, Artificial Sequence
  TCR-HT ACC4 FLRIP-8
caagcagaagacggcatacgagatcgcattagacacgtcacctatagccgcttaagtctacactacc

SEQ ID NO: 244 - DNA, 67 bp, Artificial Sequence
  TCR-HT ACC4 FLRIP-9
caagcagaagacggcatacgagatttggaccaacacgtcacctatagccgcttaagtctacactacc

SEQ ID NO: 245 - DNA, 67 bp, Artificial Sequence
  TCR-HT ACC4 FLRIP-10
caagcagaagacggcatacgagattgatgcacacacgtcacctatagccgcttaagtctacactacc

SEQ ID NO: 246 - DNA, 67 bp, Artificial Sequence
  TCR-HT ACC4 FLRIP-11
caagcagaagacggcatacgagataacgctgtacacgtcacctatagccgcttaagtctacactacc

SEQ ID NO: 247 - DNA, 67 bp, Artificial Sequence
  TCR-HT ACC4 FLRIP-12
caagcagaagacggcatacgagattgatgaccacacgtcacctatagccgcttaagtctacactacc

SEQ ID NO: 248 - DNA, 67 bp, Artificial Sequence
  TCR-HT ACC4 FLRIP-13
caagcagaagacggcatacgagatcataggtcacacgtcacctatagccgcttaagtctacactacc

SEQ ID NO: 249 - DNA, 67 bp, Artificial Sequence
  TCR-HT ACC4 FLRIP-14
caagcagaagacggcatacgagatcttcgagaacacgtcacctatagccgcttaagtctacactacc

SEQ ID NO: 250 - DNA, 67 bp, Artificial Sequence
  TCR-HT ACC4 FLRIP-15
caagcagaagacggcatacgagattactgcgaacacgtcacctatagccgcttaagtctacactacc

SEQ ID NO: 251 - DNA, 67 bp, Artificial Sequence
  TCR-HT ACC4 FLRIP-16
caagcagaagacggcatacgagatgcttagacacacgtcacctatagccgcttaagtctacactacc

SEQ ID NO: 252 - DNA, 67 bp, Artificial Sequence; identical to SEQ ID NO: 236
  TCR-HT ACC4 FLRMIP-1

SEQ ID NO: 253 - DNA, 67 bp, Artificial Sequence; identical to SEQ ID NO: 237
  TCR-HT ACC4 FLRMIP-2

SEQ ID NO: 254 - DNA, 67 bp, Artificial Sequence; identical to SEQ ID NO: 238
  TCR-HT ACC4 FLRMIP-3

SEQ ID NO: 255 - DNA, 67 bp, Artificial Sequence; identical to SEQ ID NO: 239
  TCR-HT ACC4 FLRMIP-4

SEQ ID NO: 256 - DNA, 67 bp, Artificial Sequence; identical to SEQ ID NO: 240
  TCR-HT ACC4 FLRMIP-5

SEQ ID NO: 257 - DNA, 67 bp, Artificial Sequence; identical to SEQ ID NO: 241
  TCR-HT ACC4 FLRMIP-6

SEQ ID NO: 258 - DNA, 67 bp, Artificial Sequence; identical to SEQ ID NO: 242
  TCR-HT ACC4 FLRMIP-7

SEQ ID NO: 259 - DNA, 67 bp, Artificial Sequence; identical to SEQ ID NO: 243
  TCR-HT ACC4 FLRMIP-8

SEQ ID NO: 260 - DNA, 67 bp, Artificial Sequence; identical to SEQ ID NO: 244
  TCR-HT ACC4 FLRMIP-9

SEQ ID NO: 261 - DNA, 67 bp, Artificial Sequence; identical to SEQ ID NO: 245
  TCR-HT ACC4 FLRMIP-10

SEQ ID NO: 262 - DNA, 67 bp, Artificial Sequence; identical to SEQ ID NO: 246
  TCR-HT ACC4 FLRMIP-11

SEQ ID NO: 263 - DNA, 67 bp, Artificial Sequence; identical to SEQ ID NO: 247
  TCR-HT ACC4 FLRMIP-12

SEQ ID NO: 264 - DNA, 67 bp, Artificial Sequence; identical to SEQ ID NO: 248
  TCR-HT ACC4 FLRMIP-13

SEQ ID NO: 265 - DNA, 67 bp, Artificial Sequence; identical to SEQ ID NO: 249
  TCR-HT ACC4 FLRMIP-14

SEQ ID NO: 266 - DNA, 67 bp, Artificial Sequence; identical to SEQ ID NO: 250
  TCR-HT ACC4 FLRMIP-15

SEQ ID NO: 267 - DNA, 67 bp, Artificial Sequence; identical to SEQ ID NO: 251
  TCR-HT ACC4 FLRMIP-16

SEQ ID NO: 268 - DNA, 15 bp, Artificial Sequence
  Terminal vector
gccgtcttctgcttg

SEQ ID NO: 269 - DNA, 15 bp, Artificial Sequence
  J probe ACC4 primer
ggtagtgtagactta

(19) United States
(12) Patent Application Publication
(10) Pub. No.: US 2022/0259659 A1
(43) Pub. Date: Aug. 18, 2022

(54) TARGETED HYBRID CAPTURE METHODS FOR DETERMINATION OF T CELL REPERTOIRES

(71) Applicant: RESOLUTION BIOSCIENCE, INC., Kirkland, WA (US)

(72) Inventors: Chris RAYMOND, Kirkland, WA (US); Jennifer HERNANDEZ, Kirkland, WA (US); Tristan SHAFFER, Kirkland, WA (US)

(21) Appl. No.: 17/627,535

(22) PCT Filed: Jun. 18, 2020
(86) PCT No.: PCT/US2020/038474

§ 371 Date: Jan. 14, 2022

Related U.S. Application Data
(60) Provisional application No. 62/887,938, filed on Aug. 16, 2019.

(51) Int. Cl. C12Q 1/6883; C12N 15/10

(52) U.S. Cl. CPC C12Q 1/6883; C12Q 1/6827; C12N 15/1065

(57) ABSTRACT

The present disclosure relates generally to methods for targeted hybrid capture of rearranged T cell receptors. More particularly, some embodiments relate to a method for direct and quantitative, error-corrected counting of genomic sequences for determining immune response gene repertoires.

### FIELD

The present disclosure relates generally to methods for targeted hybrid capture of rearranged T cell receptors. More particularly, some embodiments relate to a method for direct and quantitative, error-corrected counting of genomic sequences. Some embodiments also relate to specific counts of T cell populations that are present in a sample.

### BACKGROUND

T cells are integral mediators of the adaptive immune response in vertebrate organisms. They control the production of antibodies by co-stimulating B cells, and they mediate direct clearance of pathogen-infected and physiologically-defective cells by direct physical engagement between the T cell and the distressed target cell. The cell-to-cell interaction between T cells and targets is undeniably complex, yet central to the process is engagement of T cell receptors (TCRs) found on the surface of the T cell surface and major histocompatibility complex (MHC) molecules displayed on the surface of target cells. The genes encoding TCRs are assembled from a pre-existing array of possible gene segments that are present as germline sequences in all cells. During T cell development, this array is assembled by site-specific recombinases into potential T cell receptor sequences (TCRs). Those cells that produce a functional TCR that does not recognize self eventually mature and become part an individual's T cell repertoire.

The introduction of therapies that rely on the stimulation of innate T cells to treat cancers has garnered well-deserved attention. Some treated patients have experienced complete and durable responses for disease indications that previously had dismal survival prognoses. The current goal of clinical research is to understand how these T cells become activated. Similarly, in the context of clinical therapy there remains a need to determine if and when efficacious T cell populations become mobilized in the eradication of cancerous tissues.

### SUMMARY

It is therefore an aspect of this disclosure to provide methods for profiling adaptive immune response genes in a sample.

Some embodiments provided herein relate to methods of identifying a rearranged adaptive immune response gene. In some embodiments, the method comprises: obtaining a sample comprising genomic DNA; isolating genomic DNA from the sample; capturing a rearranged adaptive immune response gene from the isolated genomic DNA by sequential hybridization; amplifying the second extended sequence; and/or sequencing the second extended sequence. In some embodiments, the sequential hybridization comprises: hybridizing the genomic DNA with a first set of probes specific to a first portion of the rearranged adaptive immune response gene to generate a hybridized sequence; extending the first set of probes to generate a first extended sequence; purifying or isolating the first extended sequence; hybridizing the purified first extended sequence with a second set of probes specific to a second portion of the rearranged adaptive immune response gene; and/or extending the second set of probes to generate a second extended sequence.

In some embodiments, the sample is obtained from a tissue or a biofluid. In some embodiments, the sample is obtained from a tumor tissue, a region proximal to a tumor tissue, an organ tissue, peripheral tissue, lymph, urine, cerebral spinal fluid, a buffy coat isolate, whole blood, peripheral blood, bone marrow, amniotic fluid, breast milk, plasma, serum, aqueous humor, vitreous humor, cochlear fluid, saliva, stool, sweat, vaginal secretions, semen, bile, tears, mucus, sputum, and/or vomit. In some embodiments, the sample comprises adaptive immune cells. In some embodiments, the sample comprises one or more immune cells, such as T cells.

In some embodiments, the rearranged adaptive immune response gene is encoded by the T cell receptor (TCR) alpha gene (TRA), the TCR beta gene (TRB), the TCR delta gene (TRD), the TCR gamma gene (TRG), the antibody heavy chain gene (IGH), the kappa light chain antibody gene (IGK), and/or the lambda light chain antibody gene (IGL).

In some embodiments, the first portion of the rearranged adaptive immune response gene is a CDR3-encoding region, comprising a V, D, or J region of the rearranged adaptive immune response gene. In some embodiments, the first extended sequence is copied with T4 DNA polymerase and T4 gene 32 protein.

In some embodiments, extending is performed in a solution containing polyethylene glycol (PEG). In some embodiments, the PEG has an average molecular weight of 8000 Daltons (PEG₈₀₀₀). In some embodiments, PEG is present in an amount of 2-40% w/v, such as 2, 2.5, 3, 3.5, 4, 4.5, 5, 5.5, 6, 6.5, 7, 7.5, 8, 8.5, 9, 9.5, 10, 15, 20, 25, 30, 35, or 40% w/v, or an amount within a range defined by any two of the aforementioned values.

In some embodiments, the method further comprises fragmenting and end-repairing the genomic DNA prior to sequential hybridization. In some embodiments, the method further comprises ligating an amplification adaptor to the first extended sequence. In some embodiments, the amplifying is performed by polymerase chain reaction (PCR).

In some embodiments, the first set of probes comprises J region sequences of human TCR alpha (TRA), human TCR beta (TRB), human TCR gamma (TRG), human TCR delta (TRG), a human antibody heavy chain (IGH), a human kappa light chain antibody (IGK), and/or a human lambda light chain antibody (IGL). In some embodiments, the first set of probes comprises V region sequences of human TRA, human TRB, human TRG, human TRD, human IGH, human IGK, and/or human IGL. In some embodiments, the second set of probes comprises J region sequences of human TRA, human TRB, human TRG, human TRD, human IGH, human IGK, and/or human IGL. In some embodiments, the second set of probes comprises V region sequences of human TRA, human TRB, human TRG, human TRD, human IGH, human IGK, and/or human IGL.

In some embodiments, the first set of probes comprises a DNA sequence tag for identification of specific clones. In some embodiments, the DNA sequence tag is a nucleic acid sequence from including 2-10 nucleic acids, such as 2, 3, 4, 5, 6, 7, 8, 9, or 10 nucleic acids selected at random. In some embodiments, the DNA sequence tag includes a sequence of NN, NNN, NNNN, NNNNN, NNNNNN, NNNNNNN, NNNNNNNN, NNNNNNNNN, or NNNNNNNNNN, wherein N is A, T, G, or C. In some embodiments, the DNA sequence tags, the first and second set of probes, and the captured sequences are all used in informatic identification of clones. In some embodiments, the sample comprises a plurality of rearranged genomic sequences.

In some embodiments, the method further comprises determining the frequency of specific T cell clones, B cell clones, or both in the sample to determine a T cell immune repertoire, a B cell repertoire, or both in the sample. In some embodiments, the method further comprises profiling circulating nucleic acids, TCR repertoire, and/or Ab repertoire in a whole blood sample. In some embodiments, the profiling comprises a determination of the characteristics of a population of nucleic acids, TCR repertoire, and/or Ab repertoire in a sample.

In some embodiments, the method further comprises assessing both circulating nucleic acid and immune repertoire from a single whole blood sample. In some embodiments, an amount of single cell genomic DNA is increased by whole genome amplification prior to analysis. In some embodiments, single cell analysis is used to identify pairing between alpha and beta chain TCR within a single cell. In some embodiments, the first set of probes comprises a nucleic acid having at least 90% sequence identity to any sequence defined by any one or more of SEQ ID NOs: 62-128. In some embodiments, the second set of probes comprises a nucleic acid having at least 90% sequence identity to any sequence defined by any one or more of SEQ ID NOs: 129-227.

### BRIEF DESCRIPTION OF THE DRAWINGS

FIG. 1 depicts a schematic representation TCR gene maturation that occurs during T cell development.

FIG. 2 illustrates the nucleotide sequence (top) and inferred amino acid sequence (bottom) composition of all functional TCR chains (alpha or beta) having a conserved cysteine (C or Cys) residue contributed by the V region on one end and a conserved phenylalanine (F or Phe) residue contributed by the J region on the other end.

FIG. 3 depicts a schematic representation of steps for TCR profiling by target enrichment in one embodiment.

FIG. 4 depicts a schematic representation showing enrichment of genomic clones with J regions, as outlined in step 3 of FIG. 3.

FIG. 5 depicts a schematic representation showing purification of J region clones and primer extension, as outlined in step 4 of FIG. 3.

FIG. 6 depicts a schematic representation showing ligation of an amplification segment to J region clones and subsequent PCR amplification, as outlined in step 5 of FIG. 3.

FIG. 7 depicts a schematic representation showing hybridization of enriched J regions with V region probes, purification, and primer extension steps, as outlined in steps 6 and 7 of FIG. 3.

FIGS. 8A-8C depict schematic representations showing amplification and indexing of V-CDR3-J region containing clones from samples. FIG. 8A depicts full length forward primer (FLFP). FIG. 8B depicts sequencing of the amplification product in three steps using specific sequencing primers. FIG. 8C depicts a copy-of-a-copy of the original genomic fragment (circled).

FIG. 9 illustrates a V region probe (left) that includes a 47 nucleotide tail sequence complementary to biotinylated oligo 587, a tag, a 10 nucleotide spacer sequence, and a 40 nucleotide genomic V region sequence. FIG. 9 also illustrates a J region probe (right) that includes a 45 nucleotide tail sequence complementary to biotinylated oligo 588, a tag, and a 40 nucleotide J region probe.

FIG. 10 illustrates a heat map of TCRs for T cell repertoire data analysis. The number of clones at each of 2430 possible V/J combinations is shown, with dark regions showing low TCR numbers observed at a specific combination and bright regions showing high TCR numbers observed at a specific combination.

FIG. 11 depicts a schematic representation of germline genome (top) and rearranged T cell genome (bottom).

FIGS. 12A-12D depict schematic representations of a method of tagging and capture of all J regions with J region probes. In FIG. 12A, a majority of captured J regions are unrearranged genomic segments, with rare clones having rearranged CDR3 sequences. In The capture products are amplified to enrich for J region-containing capture clones (FIG. 12B). In FIG. 12C, a second round of capture targets V regions. The second round of capture products is amplified for sequencing (FIG. 12D).

FIGS. 13A-13B depicts a schematic representation of a read configuration. FIG. 13A shows read elements and FIG. 13B shows the observed sequence output for READ1 (SEQ ID NO: 60) and READ2 (SEQ ID NO: 61).

FIG. 14 depicts a schematic representation showing that the 3′ to 5′ exonuclease activity of T4 DNA polymerase is capable of generating a blunt end on unoccupied probes, which then becomes a substrate for ligation to the P1 adaptor sequence.

FIG. 15 depicts oligonucleotides that enable post-processing suppressive PCR, full-length amplification, and sequencing, including SEQ ID NOs: 1-10.

FIG. 16 depicts tagged V2 set probes having hexamer tags to establish independent capture events with the same sequencing start site from sibling clones that arise during post-capture amplification, and include the sequences as defined in SEQ ID NOs: 11-59.

FIG. 17 shows a gel image of raw and sonicated gDNAs used in library free experiments. F, S, C, and L represent four different gDNAs.

FIG. 18 graphically depicts an amplification plot of four library-free test samples shown in quadruplicate.

FIGS. 19A-19B show gel images from a library free amplification reaction. FIG. 19A shows a gel image of raw PCR product from library free amplification reaction. FIG. 19B shows a bead-cleaned PCR product from library free amplification reaction.

FIG. 20 shows a qPCR analysis of library-free samples libraries.

FIG. 21 graphically depicts an amplification plot, showing experiments with polymerase (P), ligase (L), or gene 32 protein (32), or combinations thereof. The combination of all three enzymes shows robust production of amplifiable library material.

FIG. 22 shows a gel image of capture PCR product with P, L, or 32, or combinations thereof. The combination of all three enzymes shows efficient production of capture PCR product.

FIG. 23 shows a gel image of individual samples of a library-free sequencing library.

FIG. 24 graphically depicts a copy number variable PLP1 in relation to the normalizing autosomal loci KRAS and MYC across samples with variable dosages of X, showing CNV for PLP1 in relation to the normalizing autosomal loci KRAS and MYC across samples with variable dosages of the X chromosome. Samples were prepared using library free methods.

FIG. 25 graphically depicts DNA sequence start points for chrX region 15 in a 4× dosage sample relative to the capture probe sequence. Reads go from left to right and samples were prepared using library free methods.

### DETAILED DESCRIPTION

Embodiments provided herein relate to methods for profiling adaptive immune response genes in a sample, including determination of adaptive immune response gene repertoires in a sample.

TCRs are a unique signature for each T cell, and therefore the determination of TCR repertoires provides direct insight into the activities of the adaptive immune response. There are several other clinical applications of TCR profiling that include minimal residual disease monitoring in T cell lymphomas, individual response to vaccines meant to stimulate the adaptive immune system, and adaptive immune responses to infectious diseases.

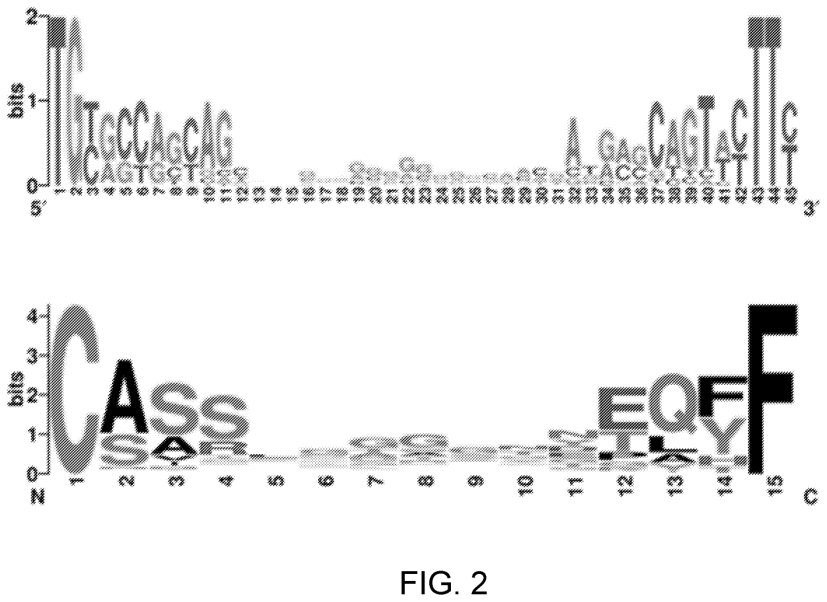

As shown in FIG. 2, the nucleotide sequence and inferred amino acid sequence composition of all functional TCR chains (alpha or beta) include a conserved cysteine (C or Cys) residue contributed by the V region on one end and a conserved phenylalanine (F or Phe) residue contributed by the J region on the other end. A “CDR3 diversity region” is the sequence in between that is unique to each CDR3.

Methods have been described in which TCR-specific PCR primers are used amplify and sequence rearranged TCR segments from genomic DNA (Robins H, Desmarais C, Matthis J, Livingston R, Andriesen J, Reijonen H, et al. Ultra-sensitive detection of rare T cell clones. J Immunol Methods. 2012 Jan. 31; 375(1-2):14-9, expressly incorporated herein by reference in its entirety). Several commercially-available methods take advantage of the fact that rearranged TCR are expressed as messenger RNAs, and they use RNA-seq methods to monitor TCR repertoires (e.g. Immunoverse from Archer Dx, Immune repertoire-seq from CD-Genomics, Full-Length V(D)J Sequences from 10× genomics). The use of molecular identifiers has been used to provide error-correction and a quantitative framework for analysis (Shugay M, Britanova O V, Merzlyak E M, Turchaninova M A, Mamedov I Z, Tuganbaev T R, et al. Towards error-free profiling of immune repertoires. Nat Methods. 2014 June; 11(6):653-5, expressly incorporated herein by reference in its entirety). Both genomic PCR and mRNA profiling, even with molecular tags, are indirect measurements of T cell repertoires. The genomic methods rely on multiplex PCR and are subject to amplification biases. Moreover, they lack error-correcting strategies and are therefore prone to over-estimates of TCR diversity. Expression-based methods measure TCR expression levels rather than T cell populations, and the well-established observation that TCR expression is governed by T cell activation (Paillard F, Sterkers G, and Vaquero C. Transcriptional and post-transcriptional regulation of TCR, CD4 and CD8 gene expression during activation of normal human T lymphocytes. EMBO J. 1990 June; 9(6): 1867-1872, expressly incorporated herein by reference in its entirety) is likely to provide a distorted view of T cell populations. This is a particularly critical consideration in the context of oncology where the efficacy of immune checkpoint inhibitors relies on a pre-existing population of inactive but potentially responsive tumor-specific killer T cells.

Some embodiments provided herein relate to a method to tag, retrieve, and/or quantify TCR repertoires. The next generation sequencing (NGS) readout is an accurate census of T cells that are present in the analysis sample. The method utilizes targeted hybrid capture technology. In the current context, tagged capture probes are used to retrieve and copy one of the partner gene segments that is rearranged to a functional TCR gene in T cells. Notably, this first capture step captures all possible gene segments, including the vast majority that is not rearranged in cells other than T cells. In a second capture step, probes specific for the other partner gene segment, which are brought in close proximity to the first partner during TCR gene development, are used to retrieve rearranged TCR genes from the initial library. In some embodiments, the method of using two capture steps is referred to herein as “sequential capture.” In some embodiments, this method provides readouts of the highly-diverse, antigen-binding CDR3 regions as a signature of individual T cells. Importantly, the TCR repertoires collected from within one individual over short periods of time may be highly similar while the repertoires collected from different individuals may differ substantially. In some embodiments, the method is both reproducible and specific.

In some embodiments, sequential capture (e.g., comprising the aforementioned two capture steps) may be used for determination of adaptive immune response gene repertoires of adaptive immune systems that undergo gene rearrangements. In some embodiments, for example, sequential capture may be used with TCR alpha and TCR beta gene targets for determination of TCR repertoires. However, the methods described herein may be used on other targets, such as other TCRs (e.g. gamma and delta chains) present on T cells that generally inhabit the digestive system. Antibody-producing B cells also possess repertoires of genes produced by genomic rearrangement. In some embodiments, methods described herein are applicable to profiling of these cell populations as well.

In some embodiments, the method of immune repertoire profiling is conducted on circulating alpha and beta chain bearing T cells. In some embodiments, the method of immune repertoire profiling is conducted on antibody producing B cells and gastric T cell delta gamma repertoires. In some embodiments, the method of immune repertoire profiling is nucleic acid hybridization and capture based. Significantly, the methods described herein differ from other profiling methods, which are PCR based. The methods described herein may use PCR to amplify DNA, but “sequential hybridization” with a first probe to one end of the TCR gene (for example, the J region or the V region), enrichment of these clones, and a second probe for the other end of the TCR (J→V, or V→J) of the enriched clones differentiates the present disclosure from standard techniques.

In addition, in some embodiments, the method for immune repertoire profiling is a genomic method that interrogates genomic DNA. In contrast, other commercially available technologies rely on mRNA transcript analysis, where mRNA is converted to cDNA and then enriched by specific PCR primers. One problem with these standards techniques is that clinicians care about T cell populations rather than expression levels of TCRs. Another issue that these standard techniques present is inaccurate test results. By way of example, consider a system having two populations of T cells, where one population is fighting off an infection. This population would be transcribing TCR message at a furious rate. The other population can fight off cancer, but the tumor is down-regulating its response. This population is making TCR message in minute quantities. If the TCR repertoire is profiled based on messenger RNA, a false conclusion would be that there are far more infection fighting cells than cancer fighting cells, even though in reality they are equal populations.

Some embodiments provided herein relate to methods that quantitatively analyze or count individual T cell clones by introducing a tag at the first hybridization step. This tag persists throughout the hybridization, capture, and sequencing steps and is used in post-sequence analysis to count T cell clones. The methods provided herein are not amenable to standard PCR-based profiling methods.

In some embodiments, these tags serve a purpose of eliminating false TCR clones. Using PCR only, it is not possible to tell the difference between a true positive clone that is rare versus a false positive clone that is the result of an error, such as a sequencing error. These false positive clones are particularly troublesome in the face of next-generation sequencing that generates millions of sequences. With the significant amount of data that is generated, errors can create functional TCR sequences that were not actually present in the biological sample being analyzed. However, the methods described herein using tags allow for identification of related sequences that arise by post-sample, error-driven processes.

Quantitative analysis of T cell clones is important for profiling T cell repertoire, and changes thereof. For example, profiling the T cell repertoire before and after an immunotherapy administration is useful for monitoring efficacy during treatment. Without wishing to be bound by theory, but by way of example, many of the newest class of immunotherapies rely on stimulating a preexisting set of TCR clones that have been inactivated by immune checkpoint molecules, such as PD-L1. By blocking the influence of PD-L1 (for example, with monoclonal antibodies), it is possible to activate the anti-tumor T cell repertoire. The course of therapy can be followed by profiling the T cell repertoire before and after administration of the PD-L1 checkpoint inhibitor. The methods described herein are useful for monitoring efficacy during methods of therapy, such as methods of treatment or inhibition of diseases such as cancer, which is valuable because some tumors respond to activation and others do not.

While still not wishing to be bound by theory, each DNA:DNA hybridization reaction is independent of a different reaction that involves a different set of sequences. By extension, it is possible to conduct thousands of probe:genomic-target capture steps simultaneously within a single reaction vessel, as long as each reaction is a simple bimolecular complex. Still further, methods described herein, including the capture methods are capable of capturing and removing TCRs, Ab-producing genes, MHC genes, tumor-related cancer genes and other adaptive immune response genes in a single reaction. In contrast, PCR-based methods rely only on the specificity of a trimolecular hybridization in which the genomic fragment, the first primer, and the second primer all specifically interact on the same genomic sequence. PCR is a far more complex reaction because subtle interactions between highly concentrated PCR primers can dominate the hybridization outcome. Thus, multiplex PCR systems are very limited and complex. The hybridization-based methods described herein operate on fundamentally different principles than existing multiplex PCR methods.

### I. Definitions

Unless defined otherwise, all technical and scientific terms used herein have the same meaning as is commonly understood by one of ordinary skill in the art. All patents, applications, published applications and other publications referenced herein are expressly incorporated by reference in their entireties unless stated otherwise. In the event that there is a plurality of definitions for a term herein, those in this section prevail unless stated otherwise.

As used herein, the term “adaptive immune system” has its ordinary meaning as understood in light of the specification, and refers to highly specialized, systemic cells and processes that eliminate pathogenic challenges. The cells of the adaptive immune system are a type of leukocyte, called a lymphocyte. B cells and T cells are the major types of lymphocytes.

As used herein, the term “immune cell” has its ordinary meaning as understood in light of the specification, and refers to cells that play a role in the immune response. Immune cells are of hematopoietic origin, and include lymphocytes, such as B cells and T cells; natural killer (NK) cells; myeloid cells, such as monocytes, macrophages, eosinophils, mast cells, basophils, and/or granulocytes.

As used herein, the term “T cell” has its ordinary meaning as understood in light of the specification, and includes CD4+ T cells and CD8+ T cells. The term T cell also includes T helper 1 type T cells, T helper 2 type T cells, T helper 17 type T cells and/or inhibitory T cells. The term “antigen presenting cell” includes antigen presenting cells (e.g., B lymphocytes, monocytes, dendritic cells, and/or Langerhans cells), as well as, other antigen presenting cells (e.g., keratinocytes, endothelial cells, astrocytes, fibroblasts, and/or oligodendrocytes). Some embodiments provided herein relate to providing or administering T cells to subjects in need of an immune response. Some embodiments provided herein relate to profiling of T cell compartments. The sorting of T cells using surface-specific markers coupled to fluorescence-activated cell sorting is a fundamental technology in immunological research. As used herein, the term “T cell compartments” has its ordinary meaning as understood in light of the specification, and refers to specific sets of T cells that all have the same surface markers.

As used herein, the term “immune response” has its ordinary meaning as understood in light of the specification, and includes T cell mediated and/or B cell mediated immune responses that are influenced by modulation of T cell co-stimulation. Exemplary immune responses include T cell responses, e.g., cytokine production, and/or cellular cytotoxicity. In addition, the term immune response includes immune responses that are indirectly affected by T cell activation, e.g., antibody production (humoral responses) and/or activation of cytokine responsive cells, e.g., macrophages. In the adaptive immune response, antigens are recognized by hypervariable molecules, such as antibodies or TCRs, which are expressed with sufficiently diverse structures to be able to recognize any antigen. Antibodies can bind to any part of the surface of an antigen. TCRs, however, are restricted to binding to short peptides bound to class I or class II molecules of the major histocompatibility complex (MHC) on the surface of APCs. TCR recognition of a peptide/MHC complex triggers activation (clonal expansion) of the T cell.

As used herein, “T cell receptor (TCR)” has its ordinary meaning as understood in light of the specification, and refers to a T cell receptor or a T cell antigen receptor, or a receptor expressed on a cell membrane of a T cell that regulates an immune system, and recognizes an antigen. There are α chain, β chain, γ chain and δ chain, constituting an αβ or γδ dimer. A TCR consisting of the former combination is called an αβ TCR and a TCR consisting of the latter combination is called a γδ TCR. T cells having such TCRs are called αβ T cell or γδ T cell. The structure is very similar to a Fab fragment of an antibody produced by a B cell, and recognizes an antigen molecule bound to an MHC molecule. Since a TCR gene of a mature T cell has undergone gene rearrangement, an individual has a diverse TCR and is able to recognize various antigens. A TCR further binds to an invariable CD3 molecule present in a cell membrane to form a complex. CD3 has an amino acid sequence called the ITAM (immunoreceptor tyrosine-based activation motif) in an intracellular region. This motif is considered to be involved in intracellular signaling. Each TCR chain is composed of a variable section (V) and a constant section (C). The constant section penetrates through the cell membrane and has a short cytoplasm portion. The variable section is present extracellularly and binds to an antigen-MHC complex. The variable section has three regions called a hypervariable section or a complementarity determining region (CDR), which binds to an antigen-MHC complex. The three CDRs are each called CDR1, CDR2, and CDR3. For a TCR, CDR1 and CDR2 are considered to bind to an MHC, while CDR3 is considered to bind to an antigen. Gene rearrangement of a TCR is similar to the process for a B cell receptor known as an immunoglobulin. In gene rearrangement of an αβ TCR, VDJ rearrangement of a β chain is first performed and then VJ rearrangement of an α chain is performed. Since a gene of a δ chain is deleted from a chromosome in rearrangement of an α chain, a T cell having an αβ TCR would not simultaneously have a γδ TCR. In contrast, in a T cell having a γδ TCR, a signal mediated by this TCR suppresses expression of a β chain. Thus, a T cell having a γδ TCR would not simultaneously have an αβ TCR.

As used herein, “B cell receptor (BCR)” has its ordinary meaning as understood in light of the specification, and is also called a B cell receptor or B cell antigen receptor and refers to those composed of an Igα/Igβ (CD79a/CD79b) heterodimer (α/β) conjugated with a membrane-bound immunoglobulin (mIg). An mIg subunit binds to an antigen to induce aggregation of the receptors, while an α/β subunit transmits a signal to the inside of a cell. BCRs, when aggregated, are understood to quickly activate Lyn, Blk, and Fyn of Src family kinases as in Syk and Btk of tyrosine kinases. Results greatly differ depending on the complexity of BCR signaling, the results including survival, resistance (allergy; lack of hypersensitivity reaction to antigen) or apoptosis, cell division, differentiation into antibody-producing cell or memory B cell and the like. Several hundred million types of T cells with a different TCR variable region sequence are produced and several hundred million types of B cells with a different BCR (or antibody) variable region sequence are produced. Individual sequences of TCRs and BCRs vary due to an introduced mutation or rearrangement of the genomic sequence. Thus, it is possible to obtain a clue for antigen specificity of a T cell or a B cell by determining a genomic sequence of TCR/BCR or a sequence of an mRNA (cDNA).

As used herein, “V region” has its ordinary meaning as understood in light of the specification, and refers to a variable section (V) of a variable region of a TCR chain or a BCR chain. As used herein, “D region” has its ordinary meaning as understood in light of the specification, and refers to a D region of a variable region of a TCR chain or a BCR chain. As used herein, “J region” has its ordinary meaning as understood in light of the specification, and refers to a J region of a variable region of a TCR chain or a BCR chain. As used herein, “C region” has its ordinary meaning as understood in light of the specification, and refers to a constant section (C) region of a TCR chain or a BCR chain.

The combinatorial joining of V and J segments in α chains and V, D and J segments in β chains produces a large number of possible molecules, thereby creating a diversity of TCRs. Diversity is also achieved in TCRs by alternative joining of gene segments. In contrast to Ig, β and δ gene segments can be joined in alternative ways. RSS flanking gene segments in β and δ gene segments can generate VJ and VDJ in the β chain, and VJ, VDJ, and VDDJ on the δ chain. As in the case of Ig, diversity is also produced by variability in the joining of gene segments. Some embodiments provided herein relate to gene segments, including T cell receptor alpha chain V region (TRAV), T cell receptor beta chain V region (TRBV) T cell receptor alpha chain J region (TRAJ), or T cell receptor beta chain J region (TRBJ).

In some embodiments, adaptive immune response genes may include TCR alpha gene (TRA), the TCR beta gene (TRB), the TCR delta gene (TRD), the TCR gamma gene (TRG), the antibody heavy chain gene (IGH), the kappa light chain antibody gene (IGK), and/or the lambda light chain antibody gene (IGL).

As used herein, the term “rearranged” has its ordinary meaning as understood in light of the specification, and refers to a configuration of a heavy chain or light chain immunoglobulin locus wherein a V segment is positioned immediately adjacent to a D-J or J segment in a conformation encoding essentially a complete VH and VL domain, respectively. A rearranged immunoglobulin gene locus can be identified by comparison to germline DNA; a rearranged locus will have at least one recombined heptamer/nonamer homology element.

As used herein, the term “unrearranged” or “germline configuration” in reference to a V segment has its ordinary meaning as understood in light of the specification, and refers to the configuration wherein the V segment is not recombined so as to be immediately adjacent to a D or J segment.

The term “gene” has its ordinary meaning as understood in light of the specification, and includes the segment of DNA involved in producing a polypeptide chain. Specifically, a gene includes, without limitation, regions preceding and following the coding region, such as the promoter and 3′-untranslated region, respectively, as well as intervening sequences (introns) between individual coding segments (exons). As used herein, “genomic DNA” refers to chromosomal DNA, as opposed to complementary DNA copied from an RNA transcript. “Genomic DNA”, as used herein, may be all of the DNA present in a single cell, or may be a portion of the DNA in a single cell.

The term “nucleic acid” or “polynucleotide” has its ordinary meaning as understood in light of the specification, and includes deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994), each of which is expressly incorporated herein by reference in its entirety). The term nucleic acid is used interchangeably with gene, cDNA, and mRNA encoded by a gene.

As used herein, the terms “nucleic acid” and “polynucleotide” are interchangeable and has its ordinary meaning as understood in light of the specification, and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate and/or sulfone linkages, or combinations of such linkages. The terms “nucleic acid” and “polynucleotide” has its ordinary meaning as understood in light of the specification, and also specifically include nucleic acids composed of bases other than the five biologically occurring bases (adenine, guanine, thymine, cytosine and uracil).

As used herein, the term “antibody” has its ordinary meaning as understood in light of the specification, and includes whole antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen.

As used herein, “CDR3” has its ordinary meaning as understood in light of the specification, and refers to the third complementarity-determining region (CDR). In this regard, CDR is a region that directly contacts an antigen and undergoes a particularly large change among variable regions, and is referred to as a hypervariable region. Each variable region of a light chain and a heavy chain has three CDRs (CDR1-CDR3) and 4 FRs (FR1-FR4) surrounding the three CDRs. Because a CDR3 region is considered to be present across V region, D region and J region, it is considered as an important key for a variable region, and is thus used as a subject of analysis. As used herein, “front of CDR3 on a reference V region” refers to a sequence corresponding to the front of CDR3 in a V region targeted by the present disclosure. As used herein, “end of CDR3 on a reference J” refers to a sequence corresponding to the end of CDR3 in a J region targeted by the present disclosure.

As used herein, the term “antigen-binding portion” of an antibody (or simply “antibody portion”), has its ordinary meaning as understood in light of the specification, and refers to one or more fragments of an antibody that retain the ability to specifically bind to an antigen (e.g., PD-1, PD-L1, and/or PD-L2). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VH, VL, CL and CH1 domains; (ii) a F(ab′)2fragment, a bivalent fragment comprising two Fab fragments linked by a disulfide bridge at the hinge region; (iii) a Fd fragment consisting of the VH and CH1 domains; (iv) a Fv fragment consisting of the VH and VL domains of a single arm of an antibody, (v) a dAb fragment, which consists of a VH domain; and (vi) an isolated complementarity determining region (CDR) or (vii) a combination of two or more isolated CDRs which may optionally be joined by a synthetic linker.

As used herein, the term “variant” has its ordinary meaning as understood in light of the specification, and refers to a polynucleotide (or polypeptide) having a sequence substantially similar to a reference polynucleotide (or polypeptide). In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least 50%, 55%, 60%, 65%, 70%, 75%, 80%, 85%, 90%, 91%, 92%, 93%, 94%, 95%, 96%, 97%, 98%, or 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least 60%, 65%, 70%, 75%, 80%, 85%, 90%, 91%, 92%, 93%, 94%, 95%, 96%, 97%, 98%, 99% or more sequence identity to the reference polypeptide as determined by sequence alignment programs known by skilled artisans.

As used herein, the term “profile” has its ordinary meaning as understood in light of the specification, and includes any set of data that represents the distinctive features or characteristics associated with a tumor, tumor cell, and/or cancer. The term encompasses a “nucleic acid profile” that analyzes one or more genetic markers, a “protein profile” that analyzes one or more biochemical or serological markers, and combinations thereof. Examples of nucleic acid profiles include, but are not limited to, a genotypic profile, gene copy number profile, gene expression profile, DNA methylation profile, and combinations thereof. Non-limiting examples of protein profiles include a protein expression profile, protein activation profile, and combinations thereof. For example, a “genotypic profile” includes a set of genotypic data that represents the genotype of one or more genes associated with a tumor, tumor cell, and/or cancer. Similarly, a “gene copy number profile” includes a set of gene copy number data that represents the amplification of one or more genes associated with a tumor, tumor cell, and/or cancer. Likewise, a “gene expression profile” includes a set of gene expression data that represents the mRNA levels of one or more genes associated with a tumor, tumor cell, and/or cancer. In addition, a “DNA methylation profile” includes a set of methylation data that represents the DNA methylation levels (e.g., methylation status) of one or more genes associated with a tumor, tumor cell, and/or cancer. Furthermore, a “protein expression profile” includes a set of protein expression data that represents the levels of one or more proteins associated with a tumor, tumor cell, and/or cancer. Moreover, a “protein activation profile” includes a set of data that represents the activation (e.g., phosphorylation status) of one or more proteins associated with a tumor, tumor cell, and/or cancer.

As used herein, “repertoire of a variable region” refers to a collection of V(D)J regions created in any manner by gene rearrangement in a TCR or BCR. The terms such as TCR repertoire and BCR repertoire are used, which are also called, for example, T cell repertoire, B cell repertoire or the like in some cases. For instance, “T cell repertoire” refers to a collection of lymphocytes characterized by expression of a T cell receptor (TCR) serving an important role in antigen recognition. A change in a T cell repertoire provides a significant indicator of an immune status in a physiological condition and disease condition. In some embodiments provided herein, a repertoire determination may include determination of a T cell immune repertoire, a B cell repertoire, circulating nucleic acids repertoire, TCR repertoire, and/or Ab repertoire.

The term “identifying” has its ordinary meaning as understood in light of the specification, and refers to assessing, determining, or ascertaining the presence, absence, identity, quality, and/or quantity of an endpoint of interest. For example, identifying a rearranged adaptive immune response gene may refer to a determination of the presence and/or quantity of an adaptive immune response gene in a sample, including a determination of the identity of the adaptive immune response gene.

The term “sample” has its ordinary meaning as understood in light of the specification, and includes any biological specimen obtained from a subject. Samples include, without limitation, a biofluid, whole blood, peripheral blood, plasma, serum, red blood cells, white blood cells (e.g., peripheral blood mononuclear cells), saliva, urine, stool, sweat, tears, vaginal secretions, nipple aspirate, amniotic fluid, breast milk, semen, bile, mucus, sputum, vomit, lymph, fine needle aspirate, cerebrospinal fluid, a buffy coat isolate, aqueous humor, vitreous humor, cochlear fluid, any other bodily fluid, bone marrow, a tissue sample, a tumor tissue, a region proximal to a tumor tissue, an organ tissue, peripheral tissue, and/or cellular extracts thereof. In some embodiments, the sample is whole blood or a fractional component thereof such as plasma, serum, or a cell pellet.

### II. T Cells

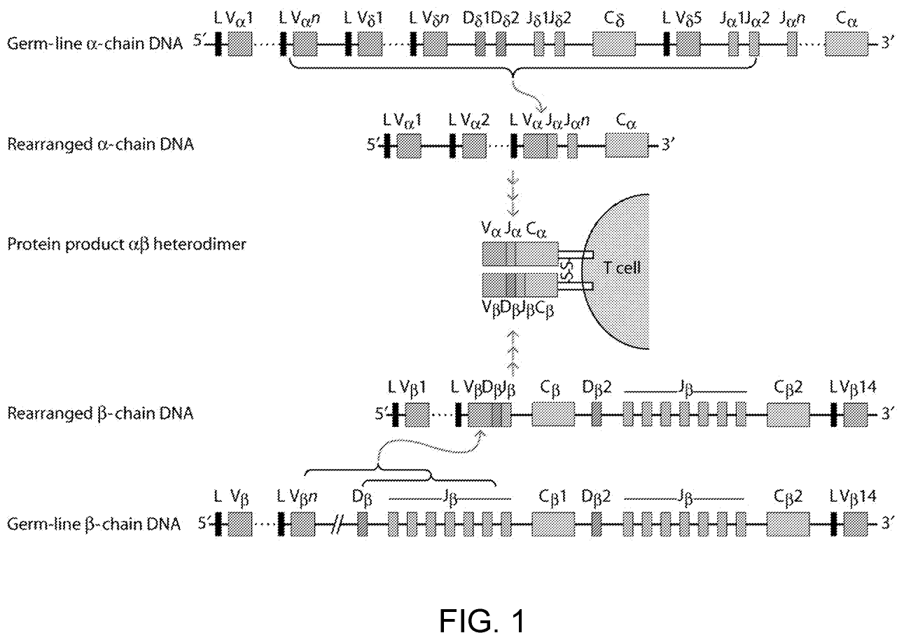

Each T cell has a unique T cell receptor (TCR). The TCRs are protein dimers on the cell surface—either α and β chains in the case of circulating T cells or γ and δ chains in T cells localized to the gut (there are yet more chains expressed during development). FIG. 1 depicts the TCR gene maturation that occurs during T cell development. These cells are part of the adaptive immune system that fights off infections and potentially cancerous cells. Therapies that activate T cells against tumors have shown great promise. B cells produce antibodies as the other major arm of the adaptive immune response. There are many clinical applications in which knowledge of B cell repertoires are also of significant utility. T cells with α and β TCRs circulate throughout the body and are responsible for fighting off cancerous cells and non-gut infections, and are relevant to oncology.

There are at least two goals to immune repertoire profiling. First, a determination the unique sequences of TCRs. The CDR3 regions are the protein segments that give each T cell its unique recognition specificity. The CDR3 coding sequence is created when V regions join with J regions. Occasionally, a small D region may exist between the V and J regions. The join between V and J is error prone by design, such that when these segments are fused, there is an intentional process where random DNA bases are inserted. This process further elaborates the TCR diversity. In some embodiments, the methods provided herein provide a determination of the DNA sequences of the V-J region across many different T cells.

Second, a count of T cell clones is determined. During an infection, certain T cell clones (as defined by their TCRs) are expanded because they are effective against an invader. Counting the numbers of each clone, even if they have the same TCR, provides a profile of the TCRs.

When genomic DNA is isolated from a sample, such as from a whole blood sample that contains T cells, for example, a molecular DNA tag is added to each genomic fragment before amplification of the genomic DNA. In this way, each TCR gene has a unique tag. Even if the TCR sequence is the same, the tag allows distinguishing of clones from different T cells versus those that are replicates from the same cell.

Normally all of the V segments and J segments are separated from one another by large, intervening genomic sequences. Only in adaptive immune response genes, such as TCR genes or antibody encoding genes, are the V and J sequences brought together in close proximity. By selecting for short genomic fragments that have both a V region and a J region on the same fragment, it is possible to enrich for functional TCR genes. A short genomic fraction can include a fraction of less than about 400 base pairs, such as less than 400, less than 350, less than 300, less than 250, less than 200, less than 150, less than 100, less than 90, less than 80, less than 70, less than 60, less than 50, or less than 40 base pairs or within a range defined by any two of the aforementioned values. Enrichment of a functional TCR gene is achieved by a sequential hybridization strategy in which all J regions are retrieved with J region specific probes. A majority of the sequences may be unrearranged, germline J segments. Following amplification of this J region enriched clone pool, fragments that also contain V regions are retrieved from the initial J pool using V region specific probes.

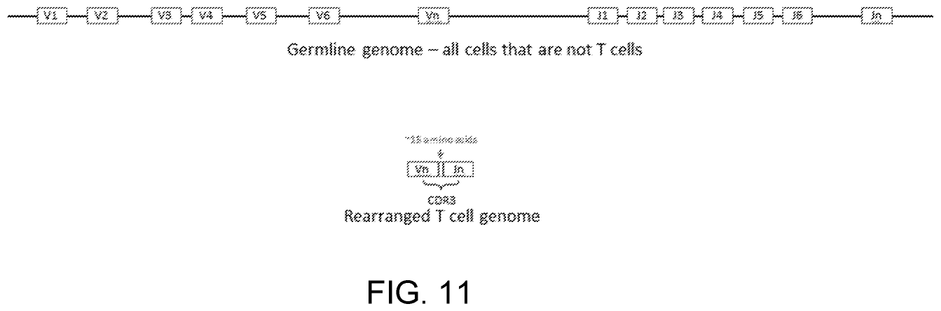

FIG. 11 illustrates differences in germline genome and rearranged T cell genome. Each T cell has a T cell receptor (TCR). The TCRs may have two chains, the α chain and the β chain. These two chains are created by similar processes where one of many V region segments is joined to one of many J region segments in a process that adds about 15 random amino acids (about 45 random nucleotides of coding sequence) between the two. The V-random-J coding region is often referred to as the CDR3 region. By counting unique CDR3 sequences, individual T cells may be counted.

### III. Target Hybrid Capture-Based TCR Enrichment

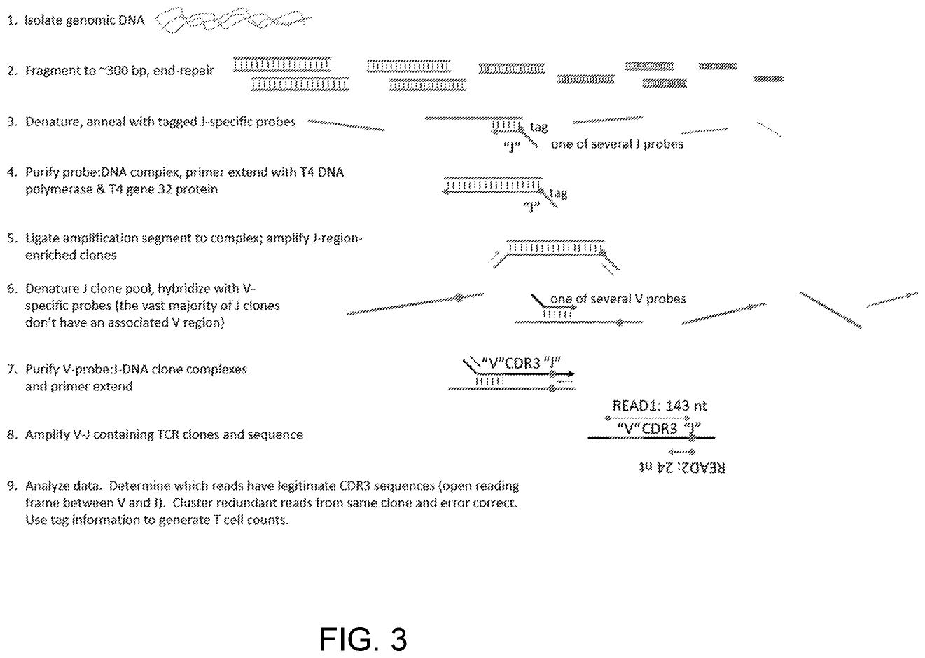

Some embodiments provided herein relate to methods and systems for target hybrid capture-based TCR enrichment. FIG. 3 schematically outlines one embodiment for target hybrid TCR enrichment. In some embodiments, the steps may include:

1. Extraction of genomic DNA from a sample. The sample is obtained from a tumor tissue, a region proximal to a tumor tissue, an organ tissue, peripheral tissue, lymph, urine, cerebral spinal fluid, a buffy coat isolate, whole blood, peripheral blood, bone marrow, amniotic fluid, breast milk, plasma, serum, aqueous humor, vitreous humor, cochlear fluid, saliva, stool, sweat, vaginal secretions, semen, bile, tears, mucus, sputum, or vomit, or any other specimen thought to contain T cells. Genomic DNA is extracted by methods known in the art, including, for example, salting-out methods, organic extraction methods, cesium chloride density gradient methods, anion-exchange methods, and silica-based methods (Green, M. R. and Sambrook J., 2012, Molecular Cloning (4th ed.), Cold Spring Harbor, N.Y.: Cold Spring Harbor Laboratory Press).

2. Fragmentation of genomic DNA to an average size of about 300 bp or 300 bp followed by end repair. Because V and J regions are normally separated by large distances (>1000 bp) in unrearranged genomes and they only move into close proximity (<100 bp) in rearranged TCR genes, this fragmentation and the subsequent demand that a fragment have both a J region and a V region heavily enriches for TCR-encoding genes. Fragmentation can be performed by standard fragmentation techniques, including, for example, shearing, sonication, or enzymatic digestion; including restriction digests, as well as other methods or combinations of these approaches. In particular embodiments, any method known in the art for fragmenting DNA can be employed with the present disclosure.

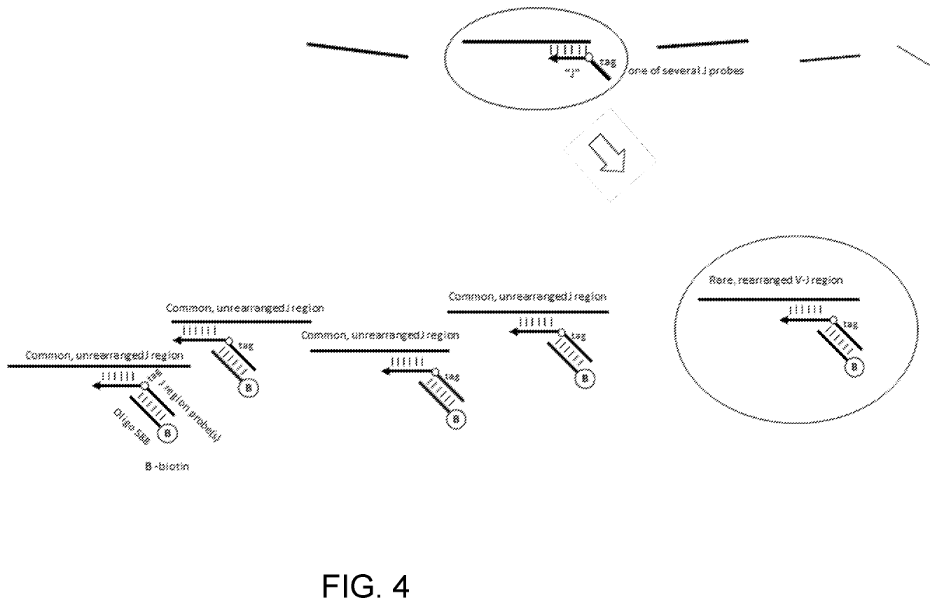

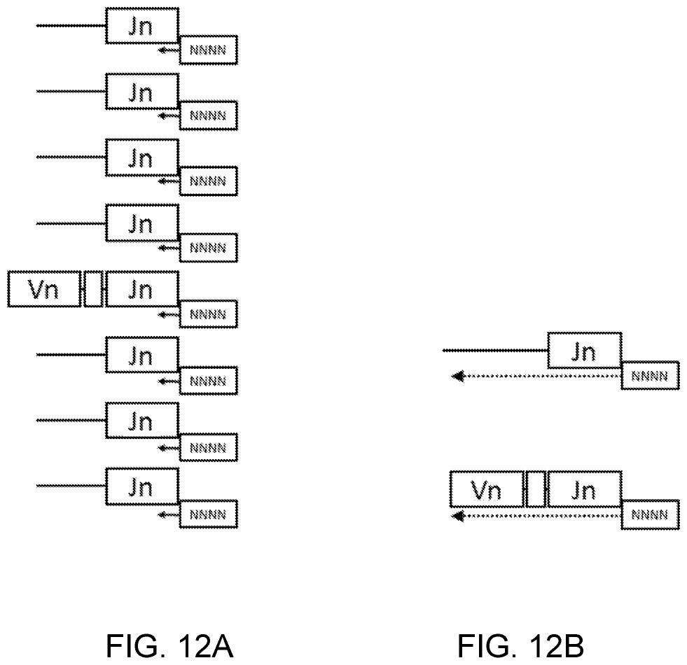

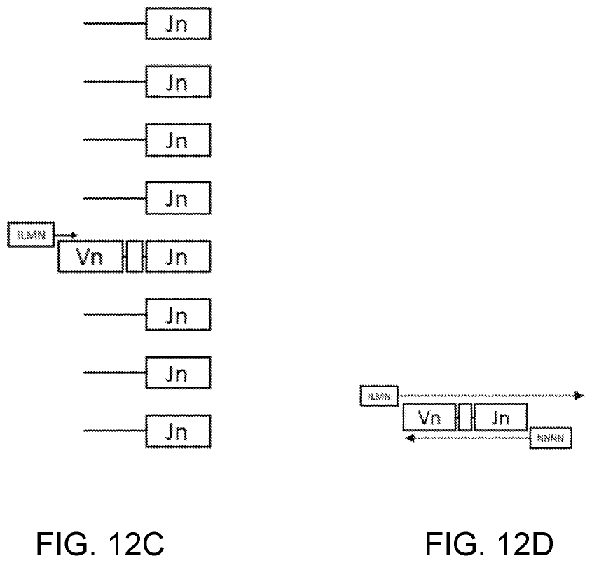

3. As shown in FIG. 4, the fragmented DNA is denatured and annealed with tagged J-specific probes. A unique molecular ID tag is included in the J region probes. In this way, every fragment that hybridizes to a J probe is uniquely marked. There are many genomic regions containing J sequences. The vast majority are un-rearranged J segments (FIG. 12A). The position of the J region within genomic fragments is variable. A rare few are rearranged J sequences in T cells. All of these J region anneal to J probes (see Table 1). Every J probe has a tag sequence. This tag sequence is important in downstream bioinformatics analysis where it is used to count T cells. Identical sequence reads with the same tag are presumed to be duplicate clones from the same original T cell. Sequence reads that have the same V-CDR3-J region sequence but a different tag are presumed to be derived from a separate T cell clone. Since T cells proliferate in response to insults, it is not unusual to find several T cells that have the exact same V-CDR3-J sequence. Primer extension creates a tagged copy of all captured J regions. Because J region probes are used first, the J probe tag (for example, a simple NNNN tetramer sequence) serves as the unique molecular identifier for TCRs.

J region probes may be 89 nt in length. They may include a 45 nt tail that is complementary to biotinylated oligo 588 (e.g., SEQ ID NO: 232). This may be followed currently by a 4 nt random sequence (NNNN). More specific and longer sequences may be used. The 40 nt J region probes may be a combination of the J coding region that comes after the conserved triplet codon for F (inclusive of the F triplet). However, the J coding region is short, so these probes also include the genomic sequences found just 3′ of the J coding regions.

The J probes may have a tail sequence that is annealed to a complementary, biotinylated sequence (e.g., 588 J-probe complement, GGTAGTGTAGACTTAAGCGGCTATAGGGACTGGTCATCGTCATCG/3BioTEG/, SEQ ID NO: 232, Table 3). The biotin moiety is used for purification by attachment of the probe:genomic DNA complex to streptavidin-coated magnetic beads.

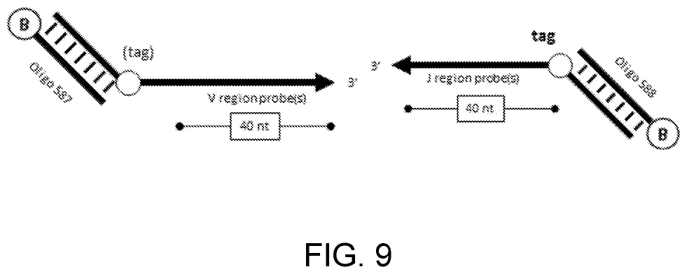

TCR J probes (FIG. 9, right) may include a 45 nucleotide tail sequence, followed by a tag of random nucleotides (e.g., NNNN), wherein N is A, T, C, or G, and wherein the tag can be 2-10 nucleotides in length, such as 2, 3, 4, 5, 6, 7, 8, 9, or 10 nucleotides in length, following by a J region probe sequences, as shown in Table 1.

TABLE 1
TCR J Probes.

| | | SEQ ID |
|---|---|---|
| TCR J Probe | Sequence | NO |
| TRAJ2_01 | CGATGACGATGACCAGTCCCTATAGCCGCTTAAGTCTACACTACCNNNN | SEQ ID |
| | ACTCACCAGATATAATGAATACATGGGTCCCTTTCCCAAA | NO: 62 |
| TRAJ3_01 | CGATGACGATGACCAGTCCCTATAGCCGCTTAAGTCTACACTACCNNNN | SEQ ID |
| | ACTTACTTGGCCGGATGCTGAGTCTGGTCCCTGATCCAAA | NO: 63 |
| TRAJ4_01 | CGATGACGATGACCAGTCCCTATAGCCGCTTAAGTCTACACTACCNNNN | SEQ ID |
| | ACTCACATGGGTGTACAGCCAGCCTGGTCCCTGCTCCAAA | NO: 64 |
| TRAJ5_01 | CGATGACGATGACCAGTCCCTATAGCCGCTTAAGTCTACACTACCNNNN | SEQ ID |
| | ACTTACTTGGTTGCACTTGGAGTCTTGTTCCACTCCCAAA | NO: 65 |
| TRAJ6_01 | CGATGACGATGACCAGTCCCTATAGCCGCTTAAGTCTACACTACCNNNN | SEQ ID |
| | ACTTACACGGATGAACAATAAGGCTGGTTCCTCTTCCAAA | NO: 66 |
| TRAJ7_01 | CGATGACGATGACCAGTCCCTATAGCCGCTTAAGTCTACACTACCNNNN | SEQ ID |
| | ACTTACTTGGTATGACCACCACTTGGTTCCCCTTCCCAAA | NO: 67 |
| TRAJ8_01 | CGATGACGATGACCAGTCCCTATAGCCGCTTAAGTCTACACTACCNNNN | SEQ ID |
| | ACTTACTTGGACTGACCAGAAGTCAGGTGCCAGTTCCAAA | NO: 68 |
| TRAJ9_01 | CGATGACGATGACCAGTCCCTATAGCCGCTTAAGTCTACACTACCNNNN | SEQ ID |
| | ACTTACTTGCTTTAACAAATAGTCTTGTTCCTGCTCCAAA | NO: 69 |
| TRAJ10_01 | CGATGACGATGACCAGTCCCTATAGCCGCTTAAGTCTACACTACCNNNN | SEQ ID |
| | ACTTACTGAGTTCCACTTTTAGCTGAGTGCCTGTCCCAAA | NO: 70 |
| TRAJ11_01 | CGATGACGATGACCAGTCCCTATAGCCGCTTAAGTCTACACTACCNNNN | SEQ ID |
| | ATGTACCTGGAGAGACTAGAAGCATAGTCCCCTTCCCAAA | NO: 71 |
| TRAJ12_01 | CGATGACGATGACCAGTCCCTATAGCCGCTTAAGTCTACACTACCNNNN | SEQ ID |
| | ACTTACCAGGCCTGACCAGCAGTCTGGTCCCACTCCCGAA | NO: 72 |
| TRAJ13_01 | CGATGACGATGACCAGTCCCTATAGCCGCTTAAGTCTACACTACCNNNN | SEQ ID |
| | ACTCACTTGGGATGACTTGGAGCTTTGTTCCAATTCCAAA | NO: 73 |
| TRAJ13_02 | CGATGACGATGACCAGTCCCTATAGCCGCTTAAGTCTACACTACCNNNN | SEQ ID |
| | ACTCACTTGGGATGACTTGGAGCTTTGTTCCAGTTCCAAA | NO: 74 |
| TRAJ14_01 | CGATGACGATGACCAGTCCCTATAGCCGCTTAAGTCTACACTACCNNNN | SEQ ID |
| | ACTTACCAGGTTTTACTGATAATCTTGTCCCACTCCCAAA | NO: 75 |
| TRAJ15_01 | CGATGACGATGACCAGTCCCTATAGCCGCTTAAGTCTACACTACCNNNN | SEQ ID |
| | ACTTACTGGAACTCACTGATAAGGTGGTTCCCTTCCCAAA | NO: 76 |
| TRAJ15_02 | CGATGACGATGACCAGTCCCTATAGCCGCTTAAGTCTACACTACCNNNN | SEQ ID |
| | ACTTACTGGAACTCACTGATAGGTGGGTTCCCTTCCCAAA | NO: 77 |
| TRAJ16_01 | CGATGACGATGACCAGTCCCTATAGCCGCTTAAGTCTACACTACCNNNN | SEQ ID |
| | ACTTACTAAGATCCACCTTTAACATGGTCCCCCTTGCAAA | NO: 78 |
| TRAJ17_01 | CGATGACGATGACCAGTCCCTATAGCCGCTTAAGTCTACACTACCNNNN | SEQ ID |
| | ACTCACTTGGTTTAACTAGCACCCTGGTTCCTCCTCCAAA | NO: 79 |
| TRAJ18_01 | CGATGACGATGACCAGTCCCTATAGCCGCTTAAGTCTACACTACCNNNN | SEQ ID |
| | ACTCACCAGGCCAGACAGTCAACTGAGTTCCTCTTCCAAA | NO: 80 |
| TRAJ20_01 | CGATGACGATGACCAGTCCCTATAGCCGCTTAAGTCTACACTACCNNNN | SEQ ID |
| | ACTTACTTGCTCTTACAGTTACTGTGGTTCCGGCTCCAAA | NO: 81 |
| TRAJ21_01 | CGATGACGATGACCAGTCCCTATAGCCGCTTAAGTCTACACTACCNNNN | SEQ ID |
| | ACTTACTTGGTTTTACATTGAGTTTGGTCCCAGATCCAAA | NO: 82 |
| TRAJ22_01 | CGATGACGATGACCAGTCCCTATAGCCGCTTAAGTCTACACTACCNNNN | SEQ ID |
| | CCAGATCCAAAGGTCAGTTGCCTTGCAGAACCAGAAGAAA | NO: 83 |
| TRAJ23_01 | CGATGACGATGACCAGTCCCTATAGCCGCTTAAGTCTACACTACCNNNN | SEQ ID |
| | ACTTACTGGGTTTCACAGATAACTCCGTTCCCTGTCCGAA | NO: 84 |
| TRAJ23_02 | CGATGACGATGACCAGTCCCTATAGCCGCTTAAGTCTACACTACCNNNN | SEQ ID |
| | ACTTACTGGGTTTCACAGATAGCTCCGTTCCCTGTCCGAA | NO: 85 |
| TRAJ24_01 | CGATGACGATGACCAGTCCCTATAGCCGCTTAAGTCTACACTACCNNNN | SEQ ID |
| | GCTTACCTGGGGTGACCACAACCTGGGTCCCTGCTCCAAA | NO: 86 |
| TRAJ26_01 | CGATGACGATGACCAGTCCCTATAGCCGCTTAAGTCTACACTACCNNNN | SEQ ID |
| | ACTTACAGGGCAGCACGGACAATCTGGTTCCGGGACCAAA | NO: 87 |
| TRAJ27_01 | CGATGACGATGACCAGTCCCTATAGCCGCTTAAGTCTACACTACCNNNN | SEQ ID |
| | ACTTACTTGGCTTCACAGTGAGCGTAGTCCCATCCCCAAA | NO: 88 |
| TRAJ28_01 | CGATGACGATGACCAGTCCCTATAGCCGCTTAAGTCTACACTACCNNNN | SEQ ID |
| | ACTTACTTGGTATGACCGAGAGTTTGGTCCCCTTCCCGAA | NO: 89 |
| TRAJ29_01 | CGATGACGATGACCAGTCCCTATAGCCGCTTAAGTCTACACTACCNNNN | SEQ ID |
| | ACTTACTTGCAATCACAGAAAGTCTTGTGCCCTTTCCAAA | NO: 90 |
| TRAJ30_01 | CGATGACGATGACCAGTCCCTATAGCCGCTTAAGTCTACACTACCNNNN | SEQ ID |
| | ACTTACTGGGGAGAATATGAAGTCGTGTCCCTTTTCCAAA | NO: 91 |
| TRAJ31_01 | CGATGACGATGACCAGTCCCTATAGCCGCTTAAGTCTACACTACCNNNN | SEQ ID |
| | ACTTACTGGGCTTCACCACCAGCTGAGTTCCATCTCCAAA | NO: 92 |
| TRAJ32_01 | CGATGACGATGACCAGTCCCTATAGCCGCTTAAGTCTACACTACCNNNN | SEQ ID |
| | ACGTACTTGGCTGGACAGCAAGCAGAGTGCCAGTTCCAAA | NO: 93 |
| TRAJ33_01 | CGATGACGATGACCAGTCCCTATAGCCGCTTAAGTCTACACTACCNNNN | SEQ ID |
| | ACTTACCTGGCTTTATAATTAGCTTGGTCCCAGCGCCCCA | NO: 94 |
| TRAJ34_01 | CGATGACGATGACCAGTCCCTATAGCCGCTTAAGTCTACACTACCNNNN | SEQ ID |
| | ACTTACTTGGAAAGACTTGTAATCTGGTCCCAGTCCCAAA | NO: 95 |
| TRAJ36_01 | CGATGACGATGACCAGTCCCTATAGCCGCTTAAGTCTACACTACCNNNN | SEQ ID |
| | ACTTACAGGGAATAACGGTGAGTCTCGTTCCAGTCCCAAA | NO: 96 |
| TRAJ37_01 | CGATGACGATGACCAGTCCCTATAGCCGCTTAAGTCTACACTACCNNNN | SEQ ID |
| | ACCTACCTGGTTTTACTTGTAAAGTTGTCCCTTGCCCAAA | NO: 97 |
| TRAJ38_01 | CGATGACGATGACCAGTCCCTATAGCCGCTTAAGTCTACACTACCNNNN | SEQ ID |
| | ACTCACTCGGATTTACTGCCAGGCTTGTTCCCAATCCCCA | NO: 98 |
| TRAJ39_01 | CGATGACGATGACCAGTCCCTATAGCCGCTTAAGTCTACACTACCNNNN | SEQ ID |
| | ACTCACGGGGTTTGACCATTAACCTTGTTCCCCCTCCAAA | NO: 99 |
| TRAJ40_01 | CGATGACGATGACCAGTCCCTATAGCCGCTTAAGTCTACACTACCNNNN | SEQ ID |
| | ACTCACTTGCTAAAACCTTCAGCCTGGTGCCTGTTCCAAA | NO: 100 |
| TRAJ41_01 | CGATGACGATGACCAGTCCCTATAGCCGCTTAAGTCTACACTACCNNNN | SEQ ID |
| | ACTCACGGGGTGTGACCAACAGCGAGGTGCCTTTGCCGAA | NO: 101 |
| TRAJ42_01 | CGATGACGATGACCAGTCCCTATAGCCGCTTAAGTCTACACTACCNNNN | SEQ ID |
| | ACTTACTTGGTTTAACAGAGAGTTTAGTGCCTTTTCCAAA | NO: 102 |
| TRAJ43_01 | CGATGACGATGACCAGTCCCTATAGCCGCTTAAGTCTACACTACCNNNN | SEQ ID |
| | ACTTACTTGGTTTTACTGTCAGTCTGGTCCCTGCTCCAAA | NO: 103 |
| TRAJ44_01 | CGATGACGATGACCAGTCCCTATAGCCGCTTAAGTCTACACTACCNNNN | SEQ ID |
| | ACCTACCGAGCGTGACCTGAAGTCTTGTTCCAGTCCCAAA | NO: 104 |
| TRAJ45_01 | CGATGACGATGACCAGTCCCTATAGCCGCTTAAGTCTACACTACCNNNN | SEQ ID |
| | ACTTACAGGGCTGGATGATTAGATGAGTCCCTTTGCCAAA | NO: 105 |
| TRAJ46_01 | CGATGACGATGACCAGTCCCTATAGCCGCTTAAGTCTACACTACCNNNN | SEQ ID |
| | ACTTACTGGGCCTAACTGCTAAACGAGTCCCGGTCCCAAA | NO: 106 |
| TRAJ47_01 | CGATGACGATGACCAGTCCCTATAGCCGCTTAAGTCTACACTACCNNNN | SEQ ID |
| | ACTCACAGGACTTGACTCTCAGAATGGTTCCTGCGCCAAA | NO: 107 |
| TRAJ48_01 | CGATGACGATGACCAGTCCCTATAGCCGCTTAAGTCTACACTACCNNNN | SEQ ID |
| | ACTTACTGGGTATGATGGTGAGTCTTGTTCCAGTCCCAAA | NO: 108 |
| TRAJ49_01 | CGATGACGATGACCAGTCCCTATAGCCGCTTAAGTCTACACTACCNNNN | SEQ ID |
| | ACTTACTTGGAATGACCGTCAAACTTGTCCCTGTCCCAAA | NO: 109 |
| TRAJ50_01 | CGATGACGATGACCAGTCCCTATAGCCGCTTAAGTCTACACTACCNNNN | SEQ ID |
| | ACTTACTTGGAATGACTGATAAGCTTGTCCCTGGCCCAAA | NO: 110 |
| TRAJ52_01 | CGATGACGATGACCAGTCCCTATAGCCGCTTAAGTCTACACTACCNNNN | SEQ ID |
| | ACTTACTTGGATGGACAGTCAAGATGGTCCCTTGTCCAAA | NO: 111 |
| TRAJ53_01 | CGATGACGATGACCAGTCCCTATAGCCGCTTAAGTCTACACTACCNNNN | SEQ ID |
| | ACTTACTTGGATTCACGGTTAAGAGAGTTCCTTTTCCAAA | NO: 112 |
| TRAJ54_01 | CGATGACGATGACCAGTCCCTATAGCCGCTTAAGTCTACACTACCNNNN | SEQ ID |
| | ACTTACTTGGGTTGATAGTCAGCCTGGTTCCTTGGCCAAA | NO: 113 |
| TRAJ56_01 | CGATGACGATGACCAGTCCCTATAGCCGCTTAAGTCTACACTACCNNNN | SEQ ID |
| | ACATACCTGGTCTAACACTCAGAGTTATTCCTTTTCCAAA | NO: 114 |
| TRAJ57_01 | CGATGACGATGACCAGTCCCTATAGCCGCTTAAGTCTACACTACCNNNN | SEQ ID |
| | ACTTACATGGGTTTACTGTCAGTTTCGTTCCCTTTCCAAA | NO: 115 |
| TRBJ1-1_V2 | CGATGACGATGACCAGTCCCTATAGCCGCTTAAGTCTACACTACCNNNN | SEQ ID |
| | ATGTCTTACCTACAACTGTGAGTCTGGTGCCTTGTCCAAA | NO: 116 |
| TRBJ1-2_V2 | CGATGACGATGACCAGTCCCTATAGCCGCTTAAGTCTACACTACCNNNN | SEQ ID |
| | CAGCCTTACCTACAACGGTTAACCTGGTCCCCGAACCGAA | NO: 117 |
| TRBJ1-3_V2 | CGATGACGATGACCAGTCCCTATAGCCGCTTAAGTCTACACTACCNNNN | SEQ ID |
| | CTTACTCACCTACAACAGTGAGCCAACTTCCCTCTCCAAA | NO: 118 |
| TRBJ1-4_V2 | CGATGACGATGACCAGTCCCTATAGCCGCTTAAGTCTACACTACCNNNN | SEQ ID |
| | TTTACATACCCAAGACAGAGAGCTGGGTTCCACTGCCAAA | NO: 119 |
| TRBJ1-5_V2 | CGATGACGATGACCAGTCCCTATAGCCGCTTAAGTCTACACTACCNNNN | SEQ ID |
| | GCAACTTACCTAGGATGGAGAGTCGAGTCCCATCACCAAA | NO: 120 |
| TRBJ1-6_V2 | CGATGACGATGACCAGTCCCTATAGCCGCTTAAGTCTACACTACCNNNN | SEQ ID |
| | CCCCCATACCTGTCACAGTGAGCCTGGTCCCGTTCCCAAA | NO: 121 |
| TRBJ2-1_V2 | CGATGACGATGACCAGTCCCTATAGCCGCTTAAGTCTACACTACCNNNN | SEQ ID |
| | CCTTCTTACCTAGCACGGTGAGCCGTGTCCCTGGCCCGAA | NO: 122 |
| TRBJ2-2_V2 | CGATGACGATGACCAGTCCCTATAGCCGCTTAAGTCTACACTACCNNNN | SEQ ID |
| | CCTCCTTACCCAGTACGGTCAGCCTAGAGCCTTCTCCAAA | NO: 123 |
| TRBJ2-3_V2 | CGATGACGATGACCAGTCCCTATAGCCGCTTAAGTCTACACTACCNNNN | SEQ ID |
| | CCCGCTTACCGAGCACTGTCAGCCGGGTGCCTGGGCCAAA | NO: 124 |
| TRBJ2-4_V2 | CGATGACGATGACCAGTCCCTATAGCCGCTTAAGTCTACACTACCNNNN | SEQ ID |
| | CCAGCTTACCCAGCACTGAGAGCCGGGTCCCGGCGCCGAA | NO: 125 |
| TRBJ2-5_V2 | CGATGACGATGACCAGTCCCTATAGCCGCTTAAGTCTACACTACCNNNN | SEQ ID |
| | CGCGCTCACCGAGCACCAGGAGCCGCGTGCCTGGCCCGAA | NO: 126 |
| TRBJ2-6_V2 | CGATGACGATGACCAGTCCCTATAGCCGCTTAAGTCTACACTACCNNNN | SEQ ID |
| | AAAACTCACCCAGCACGGTCAGCCTGCTGCCGGCCCCGAA | NO: 127 |
| TRBJ2-7_V2 | CGATGACGATGACCAGTCCCTATAGCCGCTTAAGTCTACACTACCNNNN | SEQ ID |
| | GAATCTCACCTGTGACCGTGAGCCTGGTGCCCGGCCCGAA | NO: 128 |

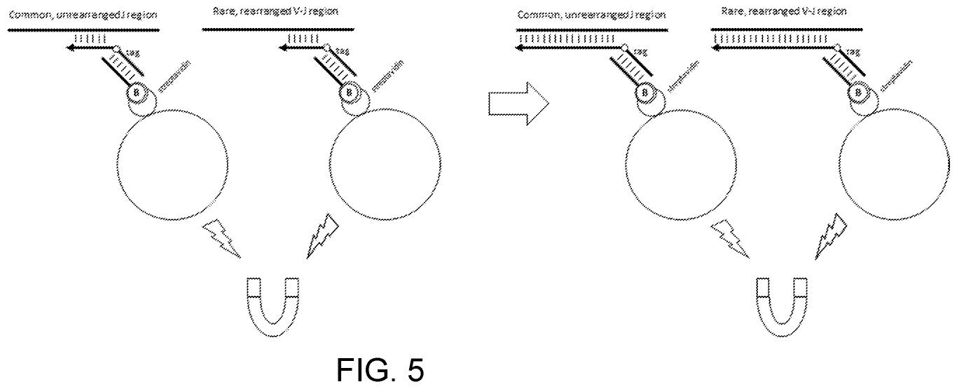

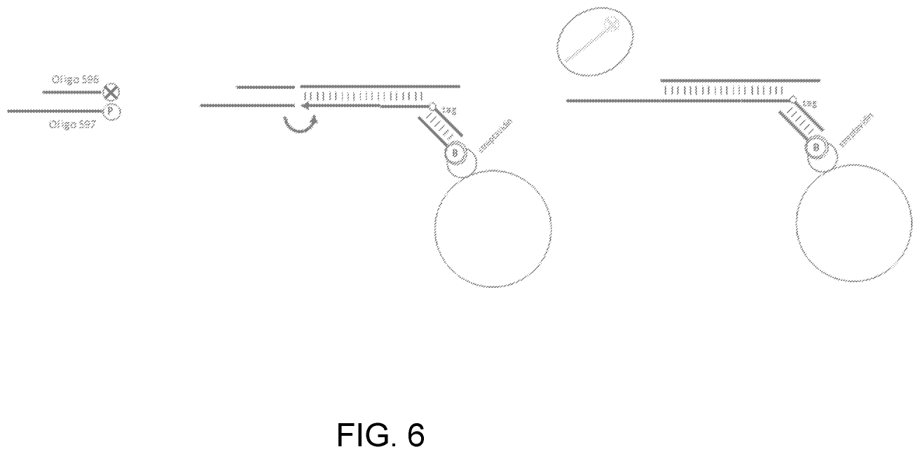

4. As shown in FIG. 5, the genomic fragments that contain a J region and are annealed to J capture probes and purified by binding to streptavidin coated magnetic beads and magnetic capture. After a wash step to remove partially annealed artifact duplexes, the J probe is extended across the captured genomic region using T4 DNA polymerase and T4 gene 32 protein in a solution that contains about 7.5% polyethylene glycol 8000 MW (PEG₈₀₀₀). This creates a blunt end that is used in a subsequent step for blunt end cloning. One of the fortuitous features here is that the reaction conditions for primer extension are also optimal for the ligation step detailed in FIG. 6. Primer extension of the J probe is somewhat unusual. The goal is to produce a perfect blunt end between the primer extended strand and the copied genomic strand (the other end probably gets filled in and becomes blunt ended as well). T4 DNA polymerase excels at making blunt ends, but it is actually a meager polymerase by itself. The addition of T4 gene 32 protein and the molecular crowding agent PEG8000 at 7.5% greatly increases the “apparent” processivity of the DNA polymerase activity (Jarvis T C, Ring D M, Daube S S, and von Hippel P H. Macromolecular crowding: thermodynamic consequences for protein-protein interactions within the T4 DNA replication complex. J Biol Chem. 1990 Sep. 5; 265(25):15160-7, expressly incorporated herein by reference in its entirety).

5. An amplification segment is ligated to J region clones and subsequently PCR amplified (FIG. 6 and FIG. 12B). To amplify the enriched J regions, a specific amplification adaptor is ligated to the extended J regions. The adaptor is a duplex of two oligonucleotides. The one that becomes attached is the phosphorylated ligation strand oligo 597 (/5Phos/GGTAGTGTAGACTTAAGCGGCTATAGG, SEQ ID NO: 234). It is duplexed to a partner oligo 596 (CCGCTTAAGTCTACACTAC/3ddC/, SEQ ID NO: 233) that is blocked on its 3′ end and therefore precluded from ligation reactivity. Following ligation, the (copied) captured J regions now have defined sequences on both ends. Moreover, these terminal sequences are an inverted repeat of the exact same sequence, meaning they can be amplified with a single primer (ACC4_27, oligo 489, CCTATAGCCGCTTAAGTCTACACTACC, SEQ ID NO: 228). Single primer amplification at this step is important to the success of the protocol because it eliminates artifacts in which the ligation adaptor ligates directly to T4 polymerase-modified probes that have no “genomic payload”. This amplification also generates enough enriched J region genomic material that it can be practically carried over to the subsequent V region probe annealing step. Without wishing to be bound by theory, it should be possible to take all hybridized J segments and move straight to the send V probe hybridization. Hence this step is “optional”. In practice, by ligating on a temporary amplification adaptor (temporary since it is lost in legitimate V-CDR3-J clones) and amplifying for 10 cycles, the yield of TCR clones greatly improves.

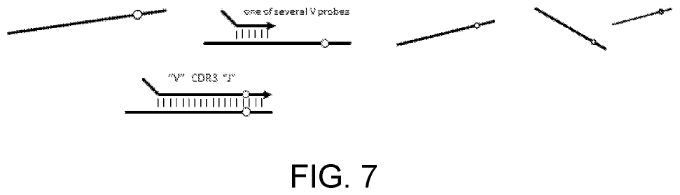

6. As shown in FIG. 7, the J clone pool is denatured and hybridized with V-specific probes (the vast majority of J clones don't have an associated V region—see FIGS. 12C and 12D).

V region probes may be 101 nt long (FIG. 9 left). From left to right they may consist of a 47 nt “tail” sequence that is complementary to a biotinylated oligonucleotide. The biotin is used for purification. This is optionally followed by a 4 nt tag. The next 10 nt may be spacer sequences for efficient sequencing. The 3′ 40 nt sequences are the genomic V region sequences that go up to the triplet coding region of the C residue.

TCR V probes may include a 45 nucleotide tail sequence, followed by a tag of random nucleotides (e.g., NNNN), wherein N is A, T, C, or G, and wherein the tag can be 2-10 nucleotides in length, such as 2, 3, 4, 5, 6, 7, 8, 9, or 10 nucleotides in length, following by a J region probe sequences, as shown in the table below.

TABLE 2
TCR V Probes.

| TCR V Probe | Sequence | SEQ ID NO |
|---|---|---|
| TRAV1-1 | AGCTCATCTGAGATGTGACTGGCACGGGAGTTGATCCTGGTTTTCACNNNN | SEQ ID |
| | ACGTCTAGACACAGGAGCTCCAGATGAAAGACTCTGCCTCTTACTTCTGC | NO: 129 |
| TRAV1-2 | AGCTCATCTGAGATGTGACTGGCACGGGAGTTGATCCTGGTTTTCACNNNN | SEQ ID |
| | CTACGCGATTGAAGGAGCTCCAGATGAAAGACTCTGCCTCTTACCTCTGT | NO: 130 |
| TRAV2 | AGCTCATCTGAGATGTGACTGGCACGGGAGTTGATCCTGGTTTTCACNNNN | SEQ ID |
| | GACATATCGGCCTCCAGGTGCGGGAGGCAGATGCTGCTGTTTACTACTGT | NO: 131 |
| TRAV3 | AGCTCATCTGAGATGTGACTGGCACGGGAGTTGATCCTGGTTTTCACNNNN | SEQ ID |
| | TGTGAGCTCAACCATCTGCCCTTGTGAGCGACTCCGCTTTGTACTTCTGT | NO: 132 |
| TRAV4 | AGCTCATCTGAGATGTGACTGGCACGGGAGTTGATCCTGGTTTTCACNNNN | SEQ ID |
| | AGATTACGGCGCCCCGGGTTTCCCTGAGCGACACTGCTGTGTACTACTGC | NO: 133 |
| TRAV5 | AGCTCATCTGAGATGTGACTGGCACGGGAGTTGATCCTGGTTTTCACNNNN | SEQ ID |
| | CATCCTGAAGTGCAGACACCCAGACTGGGGACTCAGCTATCTACTTCTGT | NO: 134 |
| TRAV6 | AGCTCATCTGAGATGTGACTGGCACGGGAGTTGATCCTGGTTTTCACNNNN | SEQ ID |
| | GTGAAGTCCTCACAGCCTCCCAGCCTGCAGACTCAGCTACCTACCTCTGT | NO: 135 |
| TRAV7 | AGCTCATCTGAGATGTGACTGGCACGGGAGTTGATCCTGGTTTTCACNNNN | SEQ ID |
| | TCCGGCATTATACAGCCGTGCAGCCTGAAGATTCAGCCACCTATTTCTGT | NO: 136 |
| TRAV8-1 | AGCTCATCTGAGATGTGACTGGCACGGGAGTTGATCCTGGTTTTCACNNNN | SEQ ID |
| | ACCGATAGCTACCCTCTGTGCAGTGGAGTGACACAGCTGAGTACTTCTGT | NO: 137 |
| TRAV8-2 | AGCTCATCTGAGATGTGACTGGCACGGGAGTTGATCCTGGTTTTCACNNNN | SEQ ID |
| | GTTAGCGATCACCCTCAGCCCATATGAGCGACGCGGCTGAGTACTTCTGT | NO: 138 |
| TRAV8-3 | AGCTCATCTGAGATGTGACTGGCACGGGAGTTGATCCTGGTTTTCACNNNN | SEQ ID |
| | CAACTGTCGAACCCTCTGTGCATTGGAGTGATGCTGCTGAGTACTTCTGT | NO: 139 |
| TRAV8-6 | AGCTCATCTGAGATGTGACTGGCACGGGAGTTGATCCTGGTTTTCACNNNN | SEQ ID |
| | TGGTCACTAGACCCTCAGTCCATATAAGCGACACGGCTGAGTACTTCTGT | NO: 140 |
| TRAV9-1 | AGCTCATCTGAGATGTGACTGGCACGGGAGTTGATCCTGGTTTTCACNNNN | SEQ ID |
| | AGCGATGTCAAGACTCAGTTCAAGAGTCAGACTCCGCTGTGTACTTCTGT | NO: 141 |
| TRAV9-2 | AGCTCATCTGAGATGTGACTGGCACGGGAGTTGATCCTGGTTTTCACNNNN | SEQ ID |
| | CTTACGACTGAGGCTCAGTTCAAGTGTCAGACTCAGCGGTGTACTTCTGT | NO: 142 |
| TRAV10 | AGCTCATCTGAGATGTGACTGGCACGGGAGTTGATCCTGGTTTTCACNNNN | SEQ ID |
| | GAGCTACAGTCACAGCCTCCCAGCTCAGCGATTCAGCCTCCTACATCTGT | NO: 143 |
| TRAV12-1 | AGCTCATCTGAGATGTGACTGGCACGGGAGTTGATCCTGGTTTTCACNNNN | SEQ ID |
| | TCATGCTGACCAGAGACTCCAAGCTCAGTGATTCAGCCACCTACCTCTGT | NO: 144 |
| TRAV12-2 | AGCTCATCTGAGATGTGACTGGCACGGGAGTTGATCCTGGTTTTCACNNNN | SEQ ID |
| | ACCTTCGAGACAGAGACTCCCAGCCCAGTGATTCAGCCACCTACCTCTGT | NO: 145 |
| TRAV12-3 | AGCTCATCTGAGATGTGACTGGCACGGGAGTTGATCCTGGTTTTCACNNNN | SEQ ID |
| | CTTCGTAGACCAGAGACTCACAGCCCAGTGATTCAGCCACCTACCTCTGT | NO: 146 |
| TRAV13-1 | AGCTCATCTGAGATGTGACTGGCACGGGAGTTGATCCTGGTTTTCACNNNN | SEQ ID |
| | GAGGAACTCTCACAGAGACCCAACCTGAAGACTCGGCTGTCTACTTCTGT | NO: 147 |
| TRAV13-2 | AGCTCATCTGAGATGTGACTGGCACGGGAGTTGATCCTGGTTTTCACNNNN | SEQ ID |
| | TGAACGTCTGTGCAGCTACTCAACCTGGAGACTCAGCTGTCTACTTTTGT | NO: 148 |
| TRAV14/DV4 | AGCTCATCTGAGATGTGACTGGCACGGGAGTTGATCCTGGTTTTCACNNNN | SEQ ID |
| | AGGACTCAGTCTCCGCTTCACAACTGGGGGACTCAGCAATGTATTTCTGT | NO: 149 |
| TRAV14/DV4 | AGCTCATCTGAGATGTGACTGGCACGGGAGTTGATCCTGGTTTTCACNNNN | SEQ ID |
| | CAAGTGTCACCTCCGCTTCACAACTGGGGGACTCAGCAATGTATTTCTGT | NO: 150 |
| TRAV16 | AGCTCATCTGAGATGTGACTGGCACGGGAGTTGATCCTGGTTTTCACNNNN | SEQ ID |
| | GTCTGAGTCAACCATTTGCTCAAGAGGAAGACTCAGCCATGTATTACTGT | NO: 151 |
| TRAV17 | AGCTCATCTGAGATGTGACTGGCACGGGAGTTGATCCTGGTTTTCACNNNN | SEQ ID |
| | TCTCACAGTGCACGGCTTCCCGGGCAGCAGACACTGCTTCTTACTTCTGT | NO: 152 |
| TRAV18 | AGCTCATCTGAGATGTGACTGGCACGGGAGTTGATCCTGGTTTTCACNNNN | SEQ ID |
| | ACCAGGATCTGCCCTCGGTGCAGCTGTCGGACTCTGCCGTGTACTACTGC | NO: 153 |
| TRAV19 | AGCTCATCTGAGATGTGACTGGCACGGGAGTTGATCCTGGTTTTCACNNNN | SEQ ID |
| | GTTGAACGTCCACAGCCTCACAAGTCGTGGACTCAGCAGTATACTTCTGT | NO: 154 |
| TRAV20 | AGCTCATCTGAGATGTGACTGGCACGGGAGTTGATCCTGGTTTTCACNNNN | SEQ ID |
| | CAGTCCTAGACACAGCCCCTAAACCTGAAGACTCAGCCACTTATCTCTGT | NO: 155 |
| TRAV21 | AGCTCATCTGAGATGTGACTGGCACGGGAGTTGATCCTGGTTTTCACNNNN | SEQ ID |
| | TGACTTGCAGTGCAGCTTCTCAGCCTGGTGACTCAGCCACCTACCTCTGT | NO: 156 |
| TRAV22 | AGCTCATCTGAGATGTGACTGGCACGGGAGTTGATCCTGGTTTTCACNNNN | SEQ ID |
| | AGGACGACTTTTCCTCTTCCCAGACCACAGACTCAGGCGTTTATTTCTGT | NO: 157 |
| TRAV23/DV6 | AGCTCATCTGAGATGTGACTGGCACGGGAGTTGATCCTGGTTTTCACNNNN | SEQ ID |
| | CTAGTACTCGCATGGATTCCCAGCCTGGAGACTCAGCCACCTACTTCTGT | NO: 158 |
| TRAV24 | AGCTCATCTGAGATGTGACTGGCACGGGAGTTGATCCTGGTTTTCACNNNN | SEQ ID |
| | GACTGCTAGACAAAGGATCCCAGCCTGAAGACTCAGCCACATACCTCTGT | NO: 159 |
| TRAV25 | AGCTCATCTGAGATGTGACTGGCACGGGAGTTGATCCTGGTTTTCACNNNN | SEQ ID |
| | TCTCATGGACCACAGCCACCCAGACTACAGATGTAGGAACCTACTTCTGT | NO: 160 |
| TRAV26-1 | AGCTCATCTGAGATGTGACTGGCACGGGAGTTGATCCTGGTTTTCACNNNN | SEQ ID |
| | ACGTTCAGCAGCCCCACGCTACGCTGAGAGACACTGCTGTGTACTATTGC | NO: 161 |
| TRAV26-2 | AGCTCATCTGAGATGTGACTGGCACGGGAGTTGATCCTGGTTTTCACNNNN | SEQ ID |
| | CTACGTTAGCGCACCGTGCTACCTTGAGAGATGCTGCTGTGTACTACTGC | NO: 162 |
| TRAV27 | AGCTCATCTGAGATGTGACTGGCACGGGAGTTGATCCTGGTTTTCACNNNN | SEQ ID |
| | GACAAGGCTTCACTGCAGCCCAGCCTGGTGATACAGGCCTCTACCTCTGT | NO: 163 |
| TRAV29/DV5 | AGCTCATCTGAGATGTGACTGGCACGGGAGTTGATCCTGGTTTTCACNNNN | SEQ ID |
| | TGTGCACTAGTGTGCCCTCCCAGCCTGGAGACTCTGCAGTGTACTTCTGT | NO: 164 |
| TRAV30 | AGCTCATCTGAGATGTGACTGGCACGGGAGTTGATCCTGGTTTTCACNNNN | SEQ ID |
| | AGAATGCCTGTACGGCCTCCCAGCTCAGTTACTCAGGAACCTACTTCTGC | NO: 165 |
| TRAV34 | AGCTCATCTGAGATGTGACTGGCACGGGAGTTGATCCTGGTTTTCACNNNN | SEQ ID |
| | CAGTCAGTCACACAGCCTCCCAGCCCAGCCATGCAGGCATCTACCTCTGT | NO: 166 |
| TRAV35 | AGCTCATCTGAGATGTGACTGGCACGGGAGTTGATCCTGGTTTTCACNNNN | SEQ ID |
| | GTTGACTAGCCTCAGCATCCATACCTAGTGATGTAGGCATCTACTTCTGT | NO: 167 |
| TRAV36/DV7 | AGCTCATCTGAGATGTGACTGGCACGGGAGTTGATCCTGGTTTTCACNNNN | SEQ ID |
| | TCCCGTAGATCACAGCCACCCAGACCGGAGACTCGGCCATCTACCTCTGT | NO: 168 |
| TRAV38-1 | AGCTCATCTGAGATGTGACTGGCACGGGAGTTGATCCTGGTTTTCACNNNN | SEQ ID |
| | ACGCTCGTAACTCAGACTCACAGCTGGGGGACACTGCGATGTATTTCTGT | NO: 169 |
| TRAV38- | AGCTCATCTGAGATGTGACTGGCACGGGAGTTGATCCTGGTTTTCACNNNN | SEQ ID |
| 2/DV8 | GTATGGACTCCTCAGACTCACAGCTGGGGGATGCCGCGATGTATTTCTGT | NO: 170 |
| TRAV39 | AGCTCATCTGAGATGTGACTGGCACGGGAGTTGATCCTGGTTTTCACNNNN | SEQ ID |
| | CACGATCAGTCACAGCTGCCGTGCATGACCTCTCTGCCACCTACTTCTGT | NO: 171 |
| TRAV40 | AGCTCATCTGAGATGTGACTGGCACGGGAGTTGATCCTGGTTTTCACNNNN | SEQ ID |
| | TGTACATGCGATATTCAGTCCAGGTATCAGACTCAGCCGTGTACTACTGT | NO: 172 |
| TRAV41 | AGCTCATCTGAGATGTGACTGGCACGGGAGTTGATCCTGGTTTTCACNNNN | SEQ ID |
| | AGACGACTTGCACAGCCTCCCATCCCAGAGACTCTGCCGTCTACATCTGT | NO: 173 |
| TRBV2_01 | AGCTCATCTGAGATGTGACTGGCACGGGAGTTGATCCTGGTTTTCACNNNN | SEQ ID |
| | TCGCCTATAGTCCGGTCCACAAAGCTGGAGGACTCAGCCATGTACTTCTG | NO: 174 |
| TRBV3-1_01 | AGCTCATCTGAGATGTGACTGGCACGGGAGTTGATCCTGGTTTTCACNNNN | SEQ ID |
| | GTCTGACAGTTCAATTCCCTGGAGCTTGGTGACTCTGCTGTGTATTTCTG | NO: 175 |
| TRBV4-1_01 | AGCTCATCTGAGATGTGACTGGCACGGGAGTTGATCCTGGTTTTCACNNNN | SEQ ID |
| | CATAAGTGCCTACACGCCCTGCAGCCAGAAGACTCAGCCCTGTATCTCTG | NO: 176 |
| TRBV4-2_01 | AGCTCATCTGAGATGTGACTGGCACGGGAGTTGATCCTGGTTTTCACNNNN | SEQ ID |
| | AGAGTCGCTATACACACCCTGCAGCCAGAAGACTCGGCCCTGTATCTCTG | NO: 177 |
| TRBV5-1_01 | AGCTCATCTGAGATGTGACTGGCACGGGAGTTGATCCTGGTTTTCACNNNN | SEQ ID |
| | TCGAACTCTGGTGAGCACCTTGGAGCTGGGGGACTCGGCCCTTTATCTTT | NO: 178 |
| TRBV5-4_01 | AGCTCATCTGAGATGTGACTGGCACGGGAGTTGATCCTGGTTTTCACNNNN | SEQ ID |
| | GTCGTGATACGTGAACGCCTTGGAGCTGGACGACTCGGCCCTGTATCTCT | NO: 179 |
| TRBV5-5_01 | AGCTCATCTGAGATGTGACTGGCACGGGAGTTGATCCTGGTTTTCACNNNN | SEQ ID |
| | CAACCTGAGTGTGAACGCCTTGTTGCTGGGGGACTCGGCCCTGTATCTCT | NO: 180 |
| TRBV5- | AGCTCATCTGAGATGTGACTGGCACGGGAGTTGATCCTGGTTTTCACNNNN | SEQ ID |
| 5_01b | AGTTGACGCAGTGAACGCCTTGTTGCTGGGGGACTCGGCCCTGTATCTCT | NO: 181 |
| TRBV5-5_01c | AGCTCATCTGAGATGTGACTGGCACGGGAGTTGATCCTGGTTTTCACNNNN | SEQ ID |
| | TCCCTGAGTAGTGAACGCCTTGTTGCTGGGGGACTCGGCCCTGTATCTCT | NO: 182 |
| TRBV5- | AGCTCATCTGAGATGTGACTGGCACGGGAGTTGATCCTGGTTTTCACNNNN | SEQ ID |
| 5_01d | GTGGACTCATGTGAACGCCTTGTTGCTGGGGGACTCGGCCCTGTATCTCT | NO: 183 |
| TRBV5-6_01 | AGCTCATCTGAGATGTGACTGGCACGGGAGTTGATCCTGGTTTTCACNNNN | SEQ ID |
| | CATAGTCAGCGTGAACGCCTTGTTGCTGGGGGACTCGGCCCTCTATCTCT | NO: 184 |
| TRBV5-8_01 | AGCTCATCTGAGATGTGACTGGCACGGGAGTTGATCCTGGTTTTCACNNNN | SEQ ID |
| | AGATCAGTCGGTGAACGCCTTGGAGCTGGAGGACTCGGCCCTGTATCTCT | NO: 185 |
| TRBV6-1_01 | AGCTCATCTGAGATGTGACTGGCACGGGAGTTGATCCTGGTTTTCACNNNN | SEQ ID |
| | TCAGCGATTCTGGAGTCGGCTGCTCCCTCCCAGACATCTGTGTACTTCTG | NO: 186 |
| TRBV6-2_01 | AGCTCATCTGAGATGTGACTGGCACGGGAGTTGATCCTGGTTTTCACNNNN | SEQ ID |
| | GTCTTCGAAGTGGAGTCGGCTGCTCCCTCCCAAACATCTGTGTACTTCTG | NO: 187 |
| TRBV6-4_01 | AGCTCATCTGAGATGTGACTGGCACGGGAGTTGATCCTGGTTTTCACNNNN | SEQ ID |
| | CATCATCGGATGGCGTCTGCTGTACCCTCTCAGACATCTGTGTACTTCTG | NO: 188 |
| TRBV6-5_01 | AGCTCATCTGAGATGTGACTGGCACGGGAGTTGATCCTGGTTTTCACNNNN | SEQ ID |
| | AGGAGATCCTTGCTGTCGGCTGCTCCCTCCCAGACATCTGTGTACTTCTG | NO: 189 |
| TRBV6-6_01 | AGCTCATCTGAGATGTGACTGGCACGGGAGTTGATCCTGGTTTTCACNNNN | SEQ ID |
| | TCCTTCGAAGTGGAGTTGGCTGCTCCCTCCCAGACATCTGTGTACTTCTG | NO: 190 |
| TRBV6-8_01 | AGCTCATCTGAGATGTGACTGGCACGGGAGTTGATCCTGGTTTTCACNNNN | SEQ ID |
| | GTGAAGCTTCTGGTGTCGGCTGCTCCCTCCCAGACATCTGTGTACTTGTG | NO: 191 |
| TRBV6-9_01 | AGCTCATCTGAGATGTGACTGGCACGGGAGTTGATCCTGGTTTTCACNNNN | SEQ ID |
| | CATGGTACCATGGAGTCAGCTGCTCCCTCCCAGACATCTGTATACTTCTG | NO: 192 |
| TRBV7-2_01 | AGCTCATCTGAGATGTGACTGGCACGGGAGTTGATCCTGGTTTTCACNNNN | SEQ ID |
| | AGACCATGGTTCCAGCGCACACAGCAGGAGGACTCGGCCGTGTATCTCTG | NO: 193 |
| TRBV7-3_01 | AGCTCATCTGAGATGTGACTGGCACGGGAGTTGATCCTGGTTTTCACNNNN | SEQ ID |
| | TCGCTGCAATTCCAGCGCACAGAGCGGGGGGACTCAGCCGTGTATCTCTG | NO: 194 |
| TRBV7-4_01 | AGCTCATCTGAGATGTGACTGGCACGGGAGTTGATCCTGGTTTTCACNNNN | SEQ ID |
| | GTTGACGCTATCCAGCGCACAGAGCAGGGGGACTCAGCTGTGTATCTCTG | NO: 195 |
| TRBV7-6_01 | AGCTCATCTGAGATGTGACTGGCACGGGAGTTGATCCTGGTTTTCACNNNN | SEQ ID |
| | CACAGATTCGTCCAGCGCACAGAGCAGCGGGACTCGGCCATGTATCGCTG | NO: 196 |
| TRBV7-7_01 | AGCTCATCTGAGATGTGACTGGCACGGGAGTTGATCCTGGTTTTCACNNNN | SEQ ID |
| | AGATCTAGGCTTCAGCGCACAGAGCAGCGGGACTCAGCCATGTATCGCTG | NO: 197 |
| TRBV7-8_01 | AGCTCATCTGAGATGTGACTGGCACGGGAGTTGATCCTGGTTTTCACNNNN | SEQ ID |
| | TCGCCATTAGTCCAGCGCACACAGCAGGAGGACTCCGCCGTGTATCTCTG | NO: 198 |
| TRBV7-9_01 | AGCTCATCTGAGATGTGACTGGCACGGGAGTTGATCCTGGTTTTCACNNNN | SEQ ID |
| | GTCGTGAATCTCCAGCGCACAGAGCAGGGGGACTCGGCCATGTATCTCTG | NO: 199 |
| TRBV9_01 | AGCTCATCTGAGATGTGACTGGCACGGGAGTTGATCCTGGTTTTCACNNNN | SEQ ID |
| | CATAACGGCTCTGAGCTCTCTGGAGCTGGGGGACTCAGCTTTGTATTTCT | NO: 200 |
| TRBV10- | AGCTCATCTGAGATGTGACTGGCACGGGAGTTGATCCTGGTTTTCACNNNN | SEQ ID |
| 1_01 | AGATGTCCGATGGAGTCTGCTGCCTCCTCCCAGACATCTGTATATTTCTG | NO: 201 |
| TRBV10- | AGCTCATCTGAGATGTGACTGGCACGGGAGTTGATCCTGGTTTTCACNNNN | SEQ ID |
| 2_01 | TCACTAGGTCTGGAGTCAGCTACCCGCTCCCAGACATCTGTGTATTTCTG | NO: 202 |
| TRBV10- | AGCTCATCTGAGATGTGACTGGCACGGGAGTTGATCCTGGTTTTCACNNNN | SEQ ID |
| 3_01 | GTTAGTCCGATGGAGTCCGCTACCAGCTCCCAGACATCTGTGTACTTCTG | NO: 203 |
| TRBV11- | AGCTCATCTGAGATGTGACTGGCACGGGAGTTGATCCTGGTTTTCACNNNN | SEQ ID |
| 1_01 | CAGTCGAACTTCCAGCCTGCAGAGCTTGGGGACTCGGCCATGTATCTCTG | NO: 204 |
| TRBV11- | AGCTCATCTGAGATGTGACTGGCACGGGAGTTGATCCTGGTTTTCACNNNN | SEQ ID |
| 2_01 | AGCGACTTAGTCCAGCCTGCAAAGCTTGAGGACTCGGCCGTGTATCTCTG | NO: 205 |
| TRBV11- | AGCTCATCTGAGATGTGACTGGCACGGGAGTTGATCCTGGTTTTCACNNNN | SEQ ID |
| 3_01 | TCGCGTCATATCCAGCCTGCAGAGCTTGGGGACTCGGCCGTGTATCTCTG | NO: 206 |
| TRBV12- | AGCTCATCTGAGATGTGACTGGCACGGGAGTTGATCCTGGTTTTCACNNNN | SEQ ID |
| 3_01 | GTTATGACGCTCCAGCCCTCAGAACCCAGGGACTCAGCTGTGTACTTCTG | NO: 207 |
| TRBV12- | AGCTCATCTGAGATGTGACTGGCACGGGAGTTGATCCTGGTTTTCACNNNN | SEQ ID |
| 5_01 | CAATACTGCGTCCAGCCCTCAGAACCCAGGGACTCAGCTGTGTATTTTTG | NO: 208 |
| TRBV13_01 | AGCTCATCTGAGATGTGACTGGCACGGGAGTTGATCCTGGTTTTCACNNNN | SEQ ID |
| | AGCGCAGTATTGAGCTCCTTGGAGCTGGGGGACTCAGCCCTGTACTTCTG | NO: 209 |
| TRBV14_01 | AGCTCATCTGAGATGTGACTGGCACGGGAGTTGATCCTGGTTTTCACNNNN | SEQ ID |
| | TCGTAGACTCTGCAGCCTGCAGAACTGGAGGATTCTGGAGTTTATTTCTG | NO: 210 |
| TRBV15_01 | AGCTCATCTGAGATGTGACTGGCACGGGAGTTGATCCTGGTTTTCACNNNN | SEQ ID |
| | GTAGTCCTGATCCGCTCACCAGGCCTGGGGGACACAGCCATGTACCTGTG | NO: 211 |
| TRBV16_01 | AGCTCATCTGAGATGTGACTGGCACGGGAGTTGATCCTGGTTTTCACNNNN | SEQ ID |
| | CATCGAGACTTCCAGGCTACGAAGCTTGAGGATTCAGCAGTGTATTTTTG | NO: 212 |
| TRBV18_01 | AGCTCATCTGAGATGTGACTGGCACGGGAGTTGATCCTGGTTTTCACNNNN | SEQ ID |
| | AGCACTTGAGTCCAGCAGGTAGTGCGAGGAGATTCGGCAGCTTATTTCTG | NO: 213 |
| TRBV19_01 | AGCTCATCTGAGATGTGACTGGCACGGGAGTTGATCCTGGTTTTCACNNNN | SEQ ID |
| | TCCAAGTTGCTGACATCGGCCCAAAAGAACCCGACAGCTTTCTATCTCTG | NO: 214 |
| TRBV20- | AGCTCATCTGAGATGTGACTGGCACGGGAGTTGATCCTGGTTTTCACNNNN | SEQ ID |
| 1_01 | GTACTCGGTACAGTGACCAGTGCCCATCCTGAAGACAGCAGCTTCTACAT | NO: 215 |
| TRBV20- | AGCTCATCTGAGATGTGACTGGCACGGGAGTTGATCCTGGTTTTCACNNNN | SEQ ID |
| 1_01b | CATGGACCATCAGTGACCAGTGCCCATCCTGAAGACAGCAGCTTCTACAT | NO: 216 |
| TRBV20- | AGCTCATCTGAGATGTGACTGGCACGGGAGTTGATCCTGGTTTTCACNNNN | SEQ ID |
| 1_01c | AGGTCTAACGCAGTGACCAGTGCCCATCCTGAAGACAGCAGCTTCTACAT | NO: 217 |
| TRBV20- | AGCTCATCTGAGATGTGACTGGCACGGGAGTTGATCCTGGTTTTCACNNNN | SEQ ID |
| 1_01d | TGAGATGCTCCAGTGACCAGTGCCCATCCTGAAGACAGCAGCTTCTACAT | NO: 218 |
| TRBV24- | AGCTCATCTGAGATGTGACTGGCACGGGAGTTGATCCTGGTTTTCACNNNN | SEQ ID |
| 1_01 | GATCTACGAGAGAGTCTGCCATCCCCAACCAGACAGCTCTTTACTTCTGT | NO: 219 |
| TRBV25- | AGCTCATCTGAGATGTGACTGGCACGGGAGTTGATCCTGGTTTTCACNNNN | SEQ ID |
| 1_01 | CTCTCGTAGATGGAGTCTGCCAGGCCCTCACATACCTCTCAGTACCTCTG | NO: 220 |
| TRBV27_01 | AGCTCATCTGAGATGTGACTGGCACGGGAGTTGATCCTGGTTTTCACNNNN | SEQ ID |
| | ACGAGCATCTTGGAGTCGCCCAGCCCCAACCAGACCTCTCTGTACTTCTG | NO: 221 |
| TRBV28_01 | AGCTCATCTGAGATGTGACTGGCACGGGAGTTGATCCTGGTTTTCACNNNN | SEQ ID |
| | TGCTTCGAAGTGGAGTCCGCCAGCACCAACCAGACATCTATGTACCTCTG | NO: 222 |
| TRBV29- | AGCTCATCTGAGATGTGACTGGCACGGGAGTTGATCCTGGTTTTCACNNNN | SEQ ID |
| 1_01 | GAGAAGCTTCCTGTGAGCAACATGAGCCCTGAAGACAGCAGCATATATCT | NO: 223 |
| TRBV29- | AGCTCATCTGAGATGTGACTGGCACGGGAGTTGATCCTGGTTTTCACNNNN | SEQ ID |
| 1_01b | CTTGGTACCACTGTGAGCAACATGAGCCCTGAAGACAGCAGCATATATCT | NO: 224 |
| TRBV29- | AGCTCATCTGAGATGTGACTGGCACGGGAGTTGATCCTGGTTTTCACNNNN | SEQ ID |
| 1_01c | ACACCATGGTCTGTGAGCAACATGAGCCCTGAAGACAGCAGCATATATCT | NO: 225 |
| TRBV29- | AGCTCATCTGAGATGTGACTGGCACGGGAGTTGATCCTGGTTTTCACNNNN | SEQ ID |
| 1_01d | TGATCACGTGCTGTGAGCAACATGAGCCCTGAAGACAGCAGCATATATCT | NO: 226 |
| TRBV30_01 | AGCTCATCTGAGATGTGACTGGCACGGGAGTTGATCCTGGTTTTCACNNNN | SEQ ID |
| | GACATGGTACGTTCTAAGAAGCTCCTTCTCAGTGACTCTGGCTTCTATCT | NO: 227 |

7. The annealed V region probes are extended. This copy of a copy is what actually is sequenced, following amplification with V probe and J probe specific primers. The temporary adaptor is lost.

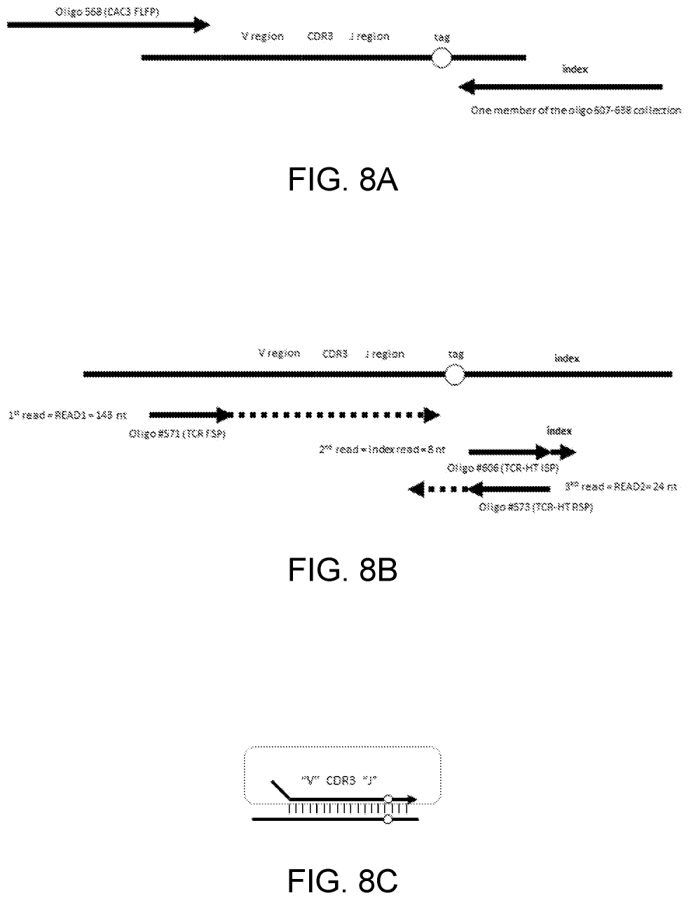

8. As shown in FIGS. 8A-8C, the V-J containing TCR clones are amplified and sequenced. In some embodiments, paired-end sequencing may be performed on an Illumina sequencer, and may consists of a longer first read and a shorter second read. The combined data provides the (potential) V-CDR3-J sequence (READ1) and the unique molecule ID tag from the J probe (READ2)

The clones are first amplified with primers that both add the sequences required for Illumina sequencing and that “index” each sample so that samples may be analyzed together. Indexing is achieved by amplifying each sample with a unique primer pair. Once the clones are amplified, they are sequenced in three separate steps using the specific sequencing primers. One PCR primer (CAC3 FLFP, oligo 568 AATGATACGGCGACCACCGAGATCTACACGTGACTGGCACGGGAGTTGATCCTG GTTTTCAC, SEQ ID NO: 229) is common to all samples. The other primer (chosen from oligos 607-638, SEQ ID NOs: 236-267) is unique to a sample and it marks each independent sample with its own “index.” In FIGS. 8A-8C, FLFP is the full length forward primer, HT is high throughput, FSP is forward sequencing primer, ISP is index sequencing primer, and RSP is reverse sequencing primer.

TABLE 3
TCR Accessory Oligonucleotides

| Oligo # | Name | Sequence | SEQ ID NO |
|---|---|---|---|
| 489 | ACC4_27 | CCTATAGCCGCTTAAGTCTACACTACC | SEQ ID NO: |
| | | | 228 |
| 568 | CAC3 FLFP | AATGATACGGCGACCACCGAGATCTACACGTGACT | SEQ ID NO: |
| | | GGCACGGGAGTTGATCCTGGTTTTCAC | 229 |
| 571 | TCR_FSP | GTGACTGGCACGGGAGTTGATCCTGGTTTTCAC | SEQ ID NO: |
| | | | 230 |
| 573 | TCR-HT_RSP | ACACGTCACCTATAGCCGCTTAAGTCTACACTACC | SEQ ID NO: |
| | | | 231 |
| 588 | J-probe complement | GGTAGTGTAGACTTAAGCGGCTATAGGGACTGGTC | SEQ ID NO: |
| | | ATCGTCATCG/3BioTEG/ | 232 |
| 596 | J-probe-part | CCGCTTAAGTCTACACTAC/3ddC/ | SEQ ID NO: |
| | | | 233 |
| 597 | J-probe-lig | /5Phos/GGTAGTGTAGACTTAAGCGGCTATAGG | SEQ ID NO: |
| | | | 234 |
| 606 | TCR-HT ISP | GGTAGTGTAGACTTAAGCGGCTATAGGTGACGTGT | SEQ ID NO: |
| | | | 235 |
| 607 | TCR-HT ACC4 FLRIP-1 | CAAGCAGAAGACGGCATACGAGATACGATGCTACA | SEQ ID NO: |
| | | CGTCACCTATAGCCGCTTAAGTCTACACTACC | 236 |
| 608 | TCR-HT ACC4 FLRIP-2 | CAAGCAGAAGACGGCATACGAGATAGTCTGACACA | SEQ ID NO: |
| | | CGTCACCTATAGCCGCTTAAGTCTACACTACC | 237 |
| 609 | TCR-HT ACC4 FLRIP-3 | CAAGCAGAAGACGGCATACGAGATCCAGGATTACA | SEQ ID NO: |
| | | CGTCACCTATAGCCGCTTAAGTCTACACTACC | 238 |
| 610 | TCR-HT ACC4 FLRIP-4 | CAAGCAGAAGACGGCATACGAGATTCGGATCAACA | SEQ ID NO: |
| | | CGTCACCTATAGCCGCTTAAGTCTACACTACC | 239 |
| 611 | TCR-HT ACC4 FLRIP-5 | CAAGCAGAAGACGGCATACGAGATAAGCCGTTACA | SEQ ID NO: |
| | | CGTCACCTATAGCCGCTTAAGTCTACACTACC | 240 |
| 612 | TCR-HT ACC4 FLRIP-6 | CAAGCAGAAGACGGCATACGAGATCACGTAGTACA | SEQ ID NO: |
| | | CGTCACCTATAGCCGCTTAAGTCTACACTACC | 241 |
| 613 | TCR-HT ACC4 FLRIP-7 | CAAGCAGAAGACGGCATACGAGATAGTCCTAGACA | SEQ ID NO: |
| | | CGTCACCTATAGCCGCTTAAGTCTACACTACC | 242 |
| 614 | TCR-HT ACC4 FLRIP-8 | CAAGCAGAAGACGGCATACGAGATCGCATTAGA | SEQ ID NO: |
| | | CACGTCACCTATAGCCGCTTAAGTCTACACTACC | 243 |
| 615 | TCR-HT ACC4 FLRIP-9 | CAAGCAGAAGACGGCATACGAGATTTGGACCAA | SEQ ID NO: |
| | | CACGTCACCTATAGCCGCTTAAGTCTACACTACC | 244 |
| 616 | TCR-HT ACC4 FLRIP-10 | CAAGCAGAAGACGGCATACGAGATTGATGCACA | SEQ ID NO: |
| | | CACGTCACCTATAGCCGCTTAAGTCTACACTACC | 245 |
| 617 | TCR-HT ACC4 FLRIP-11 | CAAGCAGAAGACGGCATACGAGATAACGCTGTA | SEQ ID NO: |
| | | CACGTCACCTATAGCCGCTTAAGTCTACACTACC | 246 |
| 618 | TCR-HT ACC4 FLRIP-12 | CAAGCAGAAGACGGCATACGAGATTGATGACCA | SEQ ID NO: |
| | | CACGTCACCTATAGCCGCTTAAGTCTACACTACC | 247 |
| 619 | TCR-HT ACC4 FLRIP-13 | CAAGCAGAAGACGGCATACGAGATCATAGGTCA | SEQ ID NO: |
| | | CACGTCACCTATAGCCGCTTAAGTCTACACTACC | 248 |
| 620 | TCR-HT ACC4 FLRIP-14 | CAAGCAGAAGACGGCATACGAGATCTTCGAGAA | SEQ ID NO: |
| | | CACGTCACCTATAGCCGCTTAAGTCTACACTACC | 249 |
| 621 | TCR-HT ACC4 FLRIP-15 | CAAGCAGAAGACGGCATACGAGATTACTGCGAA | SEQ ID NO: |
| | | CACGTCACCTATAGCCGCTTAAGTCTACACTACC | 250 |
| 622 | TCR-HT ACC4 FLRIP-16 | CAAGCAGAAGACGGCATACGAGATGCTTAGACA | SEQ ID NO: |
| | | CACGTCACCTATAGCCGCTTAAGTCTACACTACC | 251 |
| 623 | TCR-HT ACC4 FLRMIP-1 | CAAGCAGAAGACGGCATACGAGATACGATGCTA | SEQ ID NO: |
| | | CACGTCACCTATAGCCGCTTAAGTCTACACTACC | 252 |
| 624 | TCR-HT ACC4 FLRMIP-2 | CAAGCAGAAGACGGCATACGAGATAGTCTGACA | SEQ ID NO: |
| | | CACGTCACCTATAGCCGCTTAAGTCTACACTACC | 253 |
| 625 | TCR-HT ACC4 FLRMIP-3 | CAAGCAGAAGACGGCATACGAGATCCAGGATTA | SEQ ID NO: |
| | | CACGTCACCTATAGCCGCTTAAGTCTACACTACC | 254 |
| 626 | TCR-HT ACC4 FLRMIP-4 | CAAGCAGAAGACGGCATACGAGATTCGGATCAA | SEQ ID NO: |
| | | CACGTCACCTATAGCCGCTTAAGTCTACACTACC | 255 |
| 627 | TCR-HT ACC4 FLRMIP-5 | CAAGCAGAAGACGGCATACGAGATAAGCCGTTA | SEQ ID NO: |
| | | CACGTCACCTATAGCCGCTTAAGTCTACACTACC | 256 |
| 628 | TCR-HT ACC4 FLRMIP-6 | CAAGCAGAAGACGGCATACGAGATCACGTAGTA | SEQ ID NO: |
| | | CACGTCACCTATAGCCGCTTAAGTCTACACTACC | 257 |
| 629 | TCR-HT ACC4 FLRMIP-7 | CAAGCAGAAGACGGCATACGAGATAGTCCTAGA | SEQ ID NO: |
| | | CACGTCACCTATAGCCGCTTAAGTCTACACTACC | 258 |
| 630 | TCR-HT ACC4 FLRMIP-8 | CAAGCAGAAGACGGCATACGAGATCGCATTAGA | SEQ ID NO: |
| | | CACGTCACCTATAGCCGCTTAAGTCTACACTACC | 259 |
| 631 | TCR-HT ACC4 FLRMIP-9 | CAAGCAGAAGACGGCATACGAGATTTGGACCAA | SEQ ID NO: |
| | | CACGTCACCTATAGCCGCTTAAGTCTACACTACC | 260 |
| 632 | TCR-HT ACC4 FLRMIP-10 | CAAGCAGAAGACGGCATACGAGATTGATGCACA | SEQ ID NO: |
| | | CACGTCACCTATAGCCGCTTAAGTCTACACTACC | 261 |
| 633 | TCR-HT ACC4 FLRMIP-11 | CAAGCAGAAGACGGCATACGAGATAACGCTGTA | SEQ ID NO: |
| | | CACGTCACCTATAGCCGCTTAAGTCTACACTACC | 262 |
| 634 | TCR-HT ACC4 FLRMIP-12 | CAAGCAGAAGACGGCATACGAGATTGATGACCA | SEQ ID NO: |
| | | CACGTCACCTATAGCCGCTTAAGTCTACACTACC | 263 |
| 635 | TCR-HT ACC4 FLRMIP-13 | CAAGCAGAAGACGGCATACGAGATCATAGGTCA | SEQ ID NO: |
| | | CACGTCACCTATAGCCGCTTAAGTCTACACTACC | 264 |
| 636 | TCR-HT ACC4 FLRMIP-14 | CAAGCAGAAGACGGCATACGAGATCTTCGAGAA | SEQ ID NO: |
| | | CACGTCACCTATAGCCGCTTAAGTCTACACTACC | 265 |
| 637 | TCR-HT ACC4 FLRMIP-15 | CAAGCAGAAGACGGCATACGAGATTACTGCGAA | SEQ ID NO: |
| | | CACGTCACCTATAGCCGCTTAAGTCTACACTACC | 266 |
| 638 | TCR-HT ACC4 FLRMIP-16 | CAAGCAGAAGACGGCATACGAGATGCTTAGACA | SEQ ID NO: |
| | | CACGTCACCTATAGCCGCTTAAGTCTACACTACC | 267 |

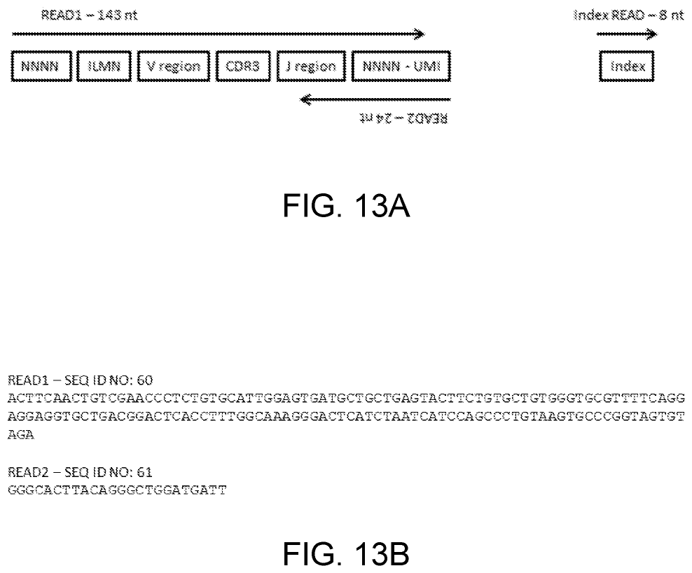

FIG. 13A represents read elements with the actual, observed sequence output shown in FIG. 13B. Most of the observed sequence is derived from probes. Reading left to right, the first four bases of READ1 is a NNNN tag. The next 10 bases are artificial spacer sequences that provide base balancing during the initial part of the sequencing run and they are unique tags for V region probes. The next 40 bases are the actual V region probe sequence. The next string of bases (averaging 45 nt but highly variable in lengths that are divisible by 3) is the core of the CDR3 sequence that is inserted during TCR genomic rearrangement. The next 40 bases are the reverse complement of the J region probe. The final bases are the reverse complement four bases UMI code and vector sequence (length permitting). The first four bases of READ2 are the UMI code followed by 20 bases of J probe sequence.

9. Informatics analysis is then performed on the sequenced clones. Embedded in the sequencing data is the T cell repertoire. “Repertoire” in this case means a quantitative listing of all observed V-CDR3-J sequences. The ID tags were added in order to enable a count of different T cells with the same TCRs as two different events. This is important when assessing an immune response, for example, a T cell response directed against a tumor that is stimulated by immunotherapy.

The overall T cell repertoire data from a single sample is large. For example, in one microgram of whole blood DNA, about 5000 different TCR alpha chain and 5000 different TCR beta chain sequences may be present. One microgram of human genomic DNA has about 167,000 diploid genomes and about 5% of the genomes present are from T cells, it is reasonable to expect to count about 8000 unique T cells (unique α+β TCRs) per analyzed sample. Many times, the exact sequence is observed multiple times, and one function of post-sequence analysis is to condense these into a unique, consensus TCR.

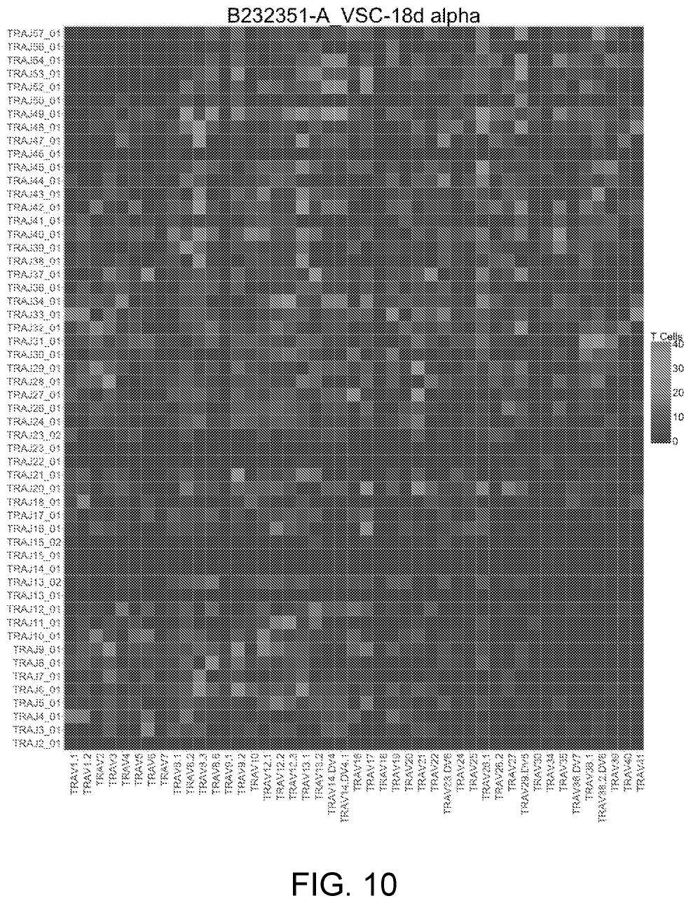

FIG. 10 illustrates an exemplary embodiment of data analysis, showing one way to display these complex datasets. Each alpha TCR is made by joining one of 45 alpha chain V regions with one of 54 possible alpha chain J regions. The heatmap in FIG. 10 shows the number of clones at each of (45×54=) 2430 possible V/J combinations. The pixel shading reflects the number of independent TCRs observed for each possible combination, with darker shading indicating fewer, and lighter shading indicating greater. The exact sequences of all the TCRs that are within each of these pixels can be retrieved.

In some embodiments, a data analysis, including a heatmap of TCRs, may be recognizable within a person's samples that are collected at intervals of weeks. Thus, in some embodiments, the T cell repertoires are reasonably stable over time. They can shift dramatically in response to an infection, a sickness, or in response to immune checkpoint blocker therapy in a cancer patient. In addition, in some embodiments, the heatmaps between different individuals are different from one another.

The primary objective of TCR analysis is counting. Each legitimate sequence is derived from a unique T cell, and the end result is census of all the T cells present in one microgram of whole blood genomic DNA.

Because each α chain is derived from the pairwise combination of 45 possible V regions and 54 possible J regions—representing a total of 2430 possible combinations—classifying the population based on the number of independent α chain clones of a particular V region that is joined to a specific J region in a table format provides a practical overview of the T cell population. Similarly, there are 45 possible β chain V regions and 12 possible β chain J regions—a total of 540 possibilities—that are also amenable to graphical display if provided in table format.

At least four elements may be taken into consideration for counting purposes. These include: 1) the J probe UMI—the first four bases of READ2; 2) the J probe sequence—the last 20 bases of READ2 (in some instances this 20 base sequence is not unique and therefore two or three α chain sequences are condensed together); 3) the V probe sequence—bases 5 through 14 of READ1 (this is the identifier that uniquely tags each V region probe; and 4) the CDR3 sequence (for example, bases 60-69 of READ1)

In addition, there are at least two kinds of artifacts in the data. The artifacts may include: 1) clones generated by probe-probe interactions, reads derived from these clones may be short and have terminal vector sequence (e.g. GCCGTCTTCTGCTTG; SEQ ID NO: 268) or they may possess J probe ACC4 primer sequences (e.g. GGTAGTGTAGACTTA; SEQ ID NO: 269). These artifacts add clones that should not be counted; and 2) clones lost because of single base read errors. The classification system described herein may include 30 error-free bases (20 for J and 10 for V) for a clone to be counted. Analyses that tolerate mismatches may increase the number of clones that are currently removed from counting consideration.

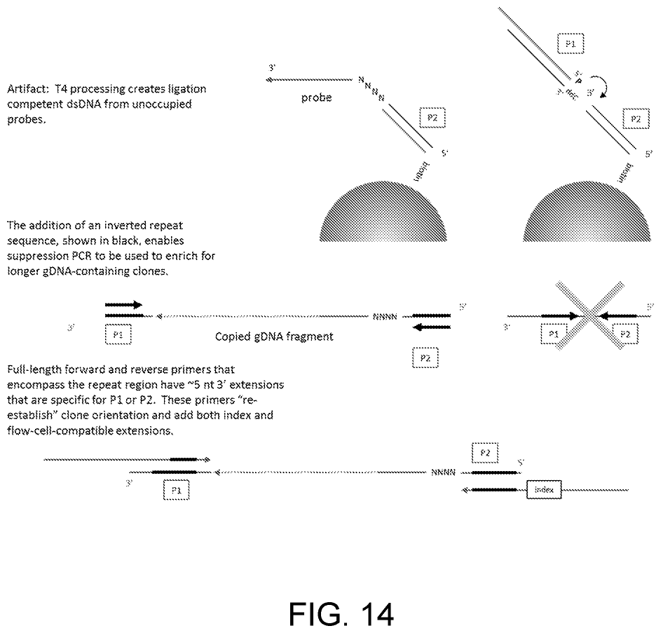

An additional artifact may occur with abundant unoccupied probes. The 3′ to 5′ exonuclease activity of T4 DNA polymerase is capable of generating a blunt end on these molecules, which then becomes a substrate for ligation to the P1 adaptor sequence (FIG. 14). These short “oligo-dimer” products will, without intervention, overwhelm the subsequent PCR reaction. To circumvent such artifacts, in some embodiments, a suppressive PCR design is included in which a 25 nt segment of P2 is included in the P1 adaptor. Following suppression PCR amplification with this segment, forward and reverse primers with P1 or P2-specific extensions may be used to add the index sequence and the flow cell-compatible extensions.

### EXAMPLES

Additional alternatives are disclosed in further detail in the following examples, which are not in any way intended to limit the scope of the claims.

### Example 1

### Library-Free Targeted Genomic Analysis

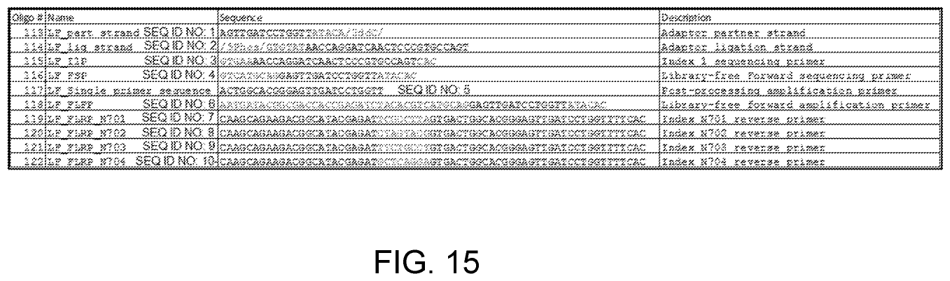

Genomic DNA samples collected from various sources were purified using the Oragene saliva collection kit. The oligonucleotides that enable post-processing suppressive PCR, full-length amplification and sequencing are shown in FIG. 15. The oligonucleotides for enabling post-processing suppressive PCR, full-length amplification, and sequencing include adaptor partner strand (SEQ ID NO: 1), adaptor ligation strand (SEQ ID NO: 2), index 1 sequencing primer (SEQ ID NO: 3), library-free forward sequencing primer (SEQ ID NO: 4), post-processing amplification primer (SEQ ID NO: 5), library-free forward amplification primer (SEQ ID NO: 6), index N701 reverse primer (SEQ ID NO: 7), index N702 reverse primer (SEQ ID NO: 8), index N703 reverse primer (SEQ ID NO: 9), and index N703 reverse primer (SEQ ID NO: 10). The samples that were sequenced in this study are shown in Table 4.

TABLE 4
Samples and Primers Used.

| Sample ID | Primer* |
|---|---|
| F | Index N701 Reverse Primer as set forth in SEQ ID NO: 7 |
| S | Index N702 Reverse Primer as set forth in SEQ ID NO: 8 |
| C | Index N703 Reverse Primer as set forth in SEQ ID NO: 9 |
| L | Index N704 Reverse Primer as set forth in SEQ ID NO: 10 |

*See FIG. 15.

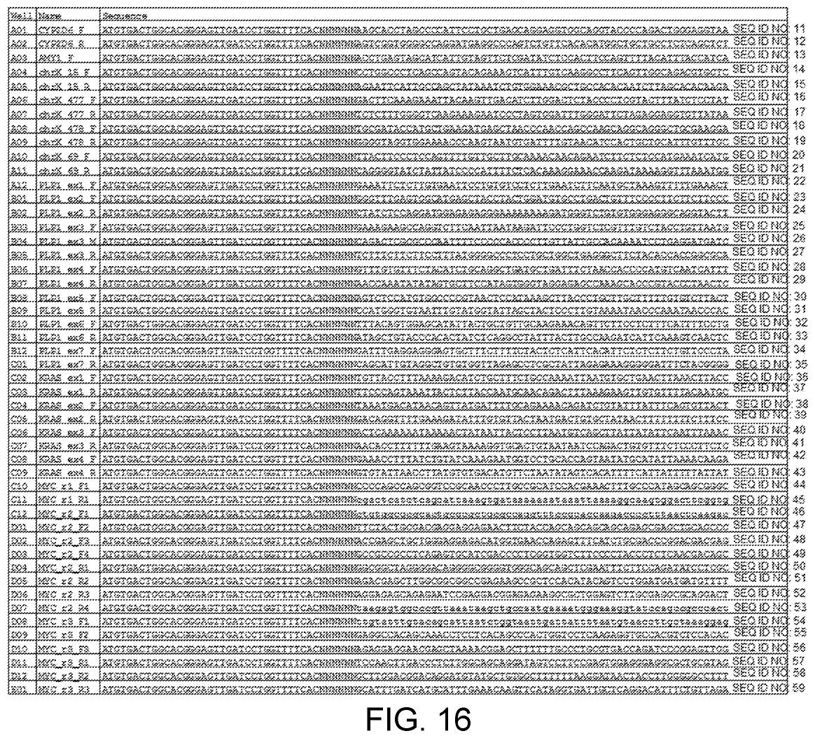

The probes are shown in FIG. 16, and are defined by the sequences set forth in SEQ ID NOs: 11-59. The hexamer tags (identified as NNNNNN, where N is A, T, C, or G) were used to establish independent capture events with the same sequencing start site from sibling clones that arose during post-capture amplification.

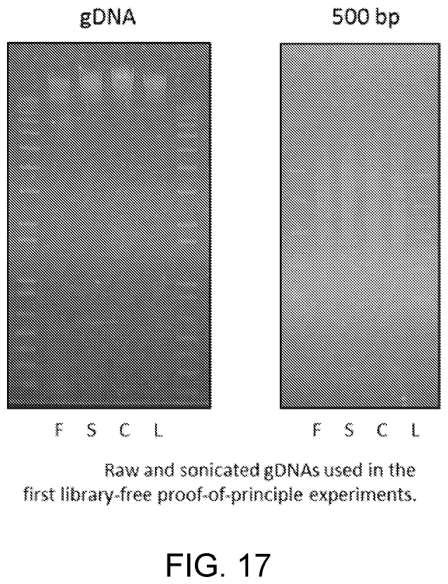

Four gDNAs (F, S, C and L) were diluted to 20 ng/μL in 150 μL final volume. The samples were sonicated to 500 bp and 125 μL was purified with 125 μL of beads. The starting material and purified, fragmented gDNA for each sample was run on a gel shown in FIG. 17. The concentrations of gDNA were 137 ng/μL (sample F), 129 ng/μL (sample S), 153 ng/μL (sample C), and 124 ng/μL (sample L).

For capture, 10 μL of gDNA sample was heated to 98° C. for 2 minutes (to achieve strand dissociation) and cooled on ice. 5 μL of 4× bind and 5 μL of the 49 probe tagged V2 probe pool (probes listed in FIG. 16) (1 nM in each probe combined with 50 nM universal oligo 61) were added and the mix was annealed (98° C. for 2 minutes followed by 4 minute incubations at successive 1° C. lower temperatures down to 69° C.). The complexes were bound to 2 μL of MyOne strep beads that were suspended in 180 μL TEzero (total volume 200 μL) for 30 minutes, washed four times, 5 minutes each with 25% formamide wash, washed once with TEzero, and the supernatants were withdrawn from the bead complexes.

For processing and adaptor ligation, 100 μL of T4 mix was made that contained: 60 μL water, 10 μL NEB “CutSmart” buffer, 15 μL 50% PEG8000, 10 μL 10 mM ATP, 1 μL 1 mM dNTP blend, 1 μL T4 gene 32 protein (NEB), and 0.5 μL T4 DNA polymerase (NEB). 25 μL of this mix was added to each of the four samples and incubated at 20° C. for 15 minutes followed by a 70° C. incubation for 10 minutes to heat inactivate the T4 polymerase. Following this 1.25 μL of adaptor (10 μM in ligation strand, pre-annealed) and 1.25 μL of HC T4 DNA ligase were added. This mixture was further incubated at 22° C. for 30 minutes and 65° C. for 10 minutes.

Here, one attractive feature of library free is that processed complexes are, at least in theory, still attached to beads. Beads were pulled from the ligation buffer and washed once with 200 μL of TEzero. The complexes were then resuspended in 2 μL. For amplification, the idea is to use single primer amplification in a 20 μL volume to both amplify target fragments and to enrich for long genomic fragments over probe “stubs”. Following this, a larger volume PCR reaction with full length primers will be used to create a “sequence-ready” library.

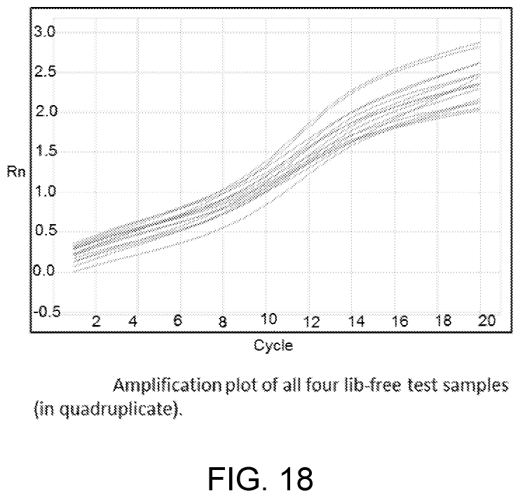

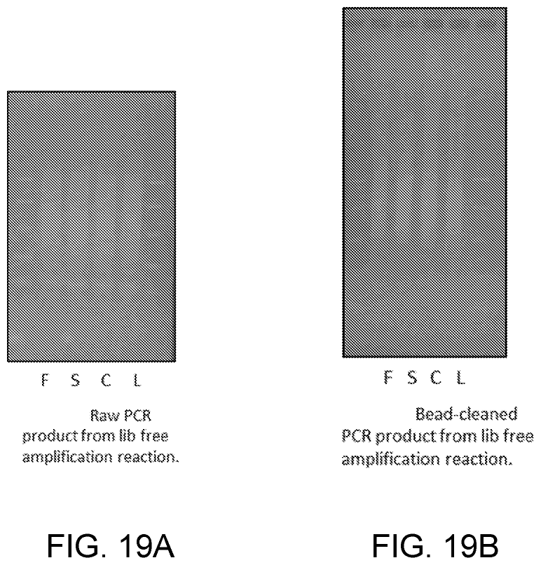

A Q5-based, single primer PCR amplification buffer was made by combining 57 μL water, 20 μL 5×Q5 reaction buffer, 10 μL of single primer 117 (see list above), 2 μL of 10 mM dNTPs, and 1 μL of Q5 hot start polymerase. Eighteen μL was added to each tube followed by amplification for 20 cycles (98° C.-30 seconds; 98° C.-10 seconds, 69° C.-10 seconds, 72° C.-10 seconds for 20 cycles; 10° C. hold). Following this, the beads were pulled out and the 20 μL of pre-amp supernatant was transferred to 280 μL of PCR mix that contained 163.5 μL water, 60 μL 5×Q5 buffer, 15 μL of forward primer 118 (10 μM), 15 μM of reverse primer 119 (10 μM), 6 μL of 10 mM dNTPs, 13.5 μL of EvaGreen+ROX dye blend (1.25 parts EG to 1 part ROX), and 3 μL of Q5 hot start polymerase (adding the dye to all reactions was unintended). Two of 100 μL aliquots were amplified by conventional PCR (98° C.-10 seconds, 69° C.-10 seconds, 72° C.-10 seconds) and quadruplicate ten μL aliquots were amplified under qPCR conditions. The amplification plot shown in FIG. 18 was observed for all four samples. It has the unusual characteristic where fluorescence began to climb immediately. The reaction seems to go through an inflection/plateau reminiscent of PCR and the conventional reactions were stopped at 20 cycles (this is now 40 total cycles of PCR). A 2% agarose gel showing the products of these amplification reactions is shown in FIG. 19A. The results were a pleasant surprise in the sense that they actually look like a sequencing library ought to look. Following bead purification (FIG. 19B) these libraries exhibited “creep”, but this was not unexpected from highly amplified libraries.

qPCR capture assays were used to determine whether gene specific targets were captured and selectively amplified. The target regions for various assays are shown in Table 2.

TABLE 2
Target Regions of qPCR Assays.

| Assay # | Target Region |
|---|---|
| 1 | PLP1 exon 2 |
| 2 | PLP1 exon 2 |
| 3 | PLP1 exon 2 |
| 4 | PLP1 upstream of exon 2 |
| 5 | PLP1 downstream of exon 2 |
| 6 | PLP1 200 bp downstream of exon 2 |
| 7 | PLP1 exon 3 |
| 8 | chr 9 off-target |
| 9 | CYP2D6 |
| 10 | chrX-154376051 |
| 11 | chrX-154376051 |
| 12 | chrX-692964 |
| 13 | KRAS region 1 |
| 14 | KRAS region 2 |
| 15 | MYC region 2 |
| 16 | MYC region 2 |

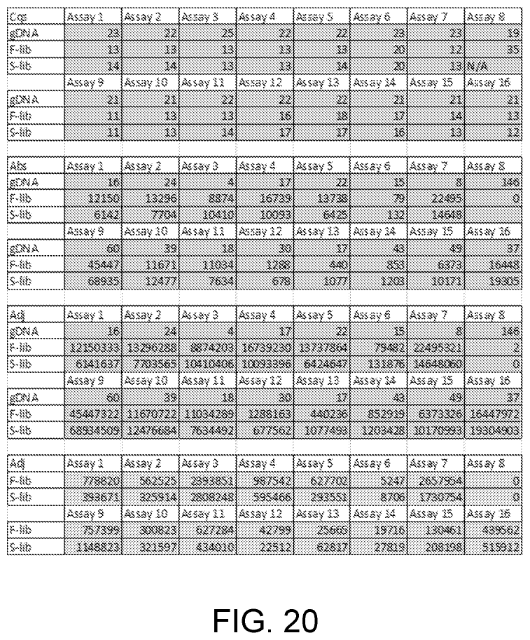

For qPCR analysis, genomic DNA from sample F at 10 ng/μL (2 μL is added to 8 μL of PCR mix to give a final volume and concentration of 10 μL and 2 ng/μL, respectively) was used as control. Purified processed material from the F and S samples was diluted to 0.01 ng/μL=10 pg/μL and 2 μL was added to each 8 μL PCR reaction to give a final concentration of 2 pg/μL. These are more or less standard qPCR assay conditions to evaluate any capture reaction. The results are shown in FIG. 20.

To this point, library-free was a collection of promising-looking smears. The qPCR data indicates that the technology is in fact very effective at retrieving the targeted genomic regions and at leaving off-target regions behind (Assays 6, 8). The fold purifications, often >500,000-fold, are directly comparable to our SOP technology.

### Example 2

### Production of Amplifiable Library Material

The results from the preliminary investigation described in Example 1 were sufficiently compelling for investigation of the enzymatic requirements for complex processing. The design of experiment is shown in Table 3.

TABLE 3
Experimental Design.

| Experiment | 1 | 2 | 3 | 4 | 5 |
|---|---|---|---|---|---|
| T4 DNA Polymerase | no | no | yes | yes | yes |
| T4 Gene 32 Protein | no | yes | no | yes | yes |
| T4 DNA Ligase | no | yes | yes | no | yes |

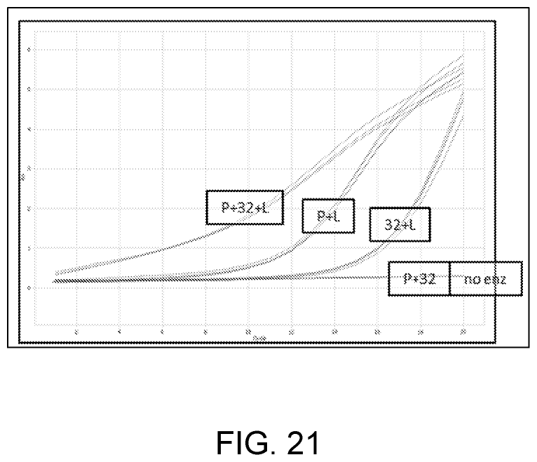

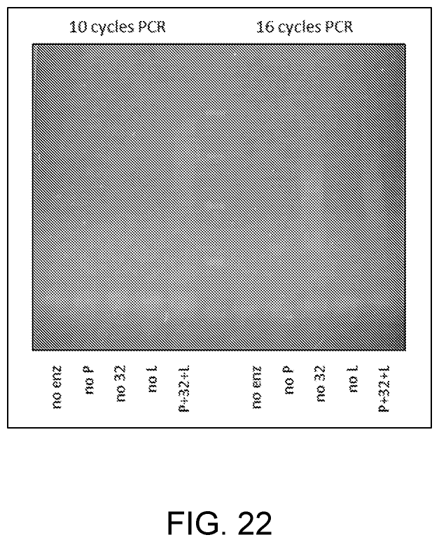

To make capture complexes for analysis, twelve identical reactions were created. Ten μL of 135 ng/μL sonicated gDNA was melted, annealed with tagged V2 probe, stuck to strep coated beads, washed and resuspended in TEzero as described above. Five hundred μL of processing master mix was prepared by combining 270 μL water, 50 μL 10× CutSmart buffer, 50 μL of 10 mM ATP, 75 μL of 50% PEG8000, and 5 μL of 10 mM dNTPs. This buffer was divided into 10 of 90 μL aliquots (duplicate tests were performed) and enzyme was added in the amounts described above (per 90 μL of master mix was added 1 μL of T4 gene 32 protein, 0.5 μL of T4 polymerase, 5 μL of adaptor and/or 5 μL of HC T4 ligase). Following T4 fill-in and ligation as described above, the complexes were washed free of processing mix in TEzero and resuspended in 2 μL TEzero. Complexes were resuspended in 20 μL final volume each of single primer amplification mix and amplified for 20 cycles as described above. The beads were then pulled aside using a magnet and the 20 μL clarified amplification was diluted into 180 μL of full-length F+R (118+119) PCR amplification mix. Fifty μL was pulled aside for qPCR analysis and the remaining 150 μL was split in two and amplified by conventional PCR. The 50 μL qPCR samples were mixed with 2.5 μL of dye blend and 10 μL aliquots were monitored by fluorescence change. The traces of this experiment are shown in FIG. 21. All three enzymes are required for robust production of amplifiable library material. One of the two conventional PCR aliquots was pulled at 10 cycles and the other at 16 cycles of PCR. Aliquots of these raw PCR reactions (5 μL of each reaction) were analyzed on 2% agarose gels. The results are shown in the gel on the following page. The striking result is that all three enzymes are required for the efficient production of amplifiable library material. The more subtle result is that the size distribution of all-three-enzyme-material at 10 cycles is significantly larger than the size distribution of P+L alone that appears at 16 cycles. This is in keeping with research literature suggesting that gene 32 protein assists in processivity and in replication through secondary structures. The fact that the P+L and L alone reactions possess any apparent primer adaptor dimer is also striking given that these reactions went through 20 cycles of highly suppressive PCR. The observation that “primer-dimer” is present would suggest that the vast majority of P+L (no gene 32) product is dimer and not copied genomic clones. These data together with the qPCR from the initial investigation argue that T4 DNA polymerase in conjunction with T4 gene 32 protein in the presence of the molecular crowding agent PEG₈₀₀₀ (the latter contribution has not been evaluated) is capable of efficiently copying captured genomic material onto capture probes.

### Example 3

### Generation of a Library-Free Sequencing Library

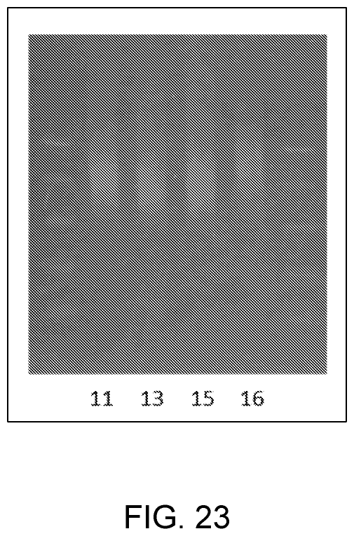

The methods described in Examples 1 and 2 were used to produce a DNA sequencing library with the four Coriell samples. Each one of the four samples was coded with an individual index code in the final PCR step. The creation of such libraries highlights that library-free methods demand that all samples in a collection be processed separately, which is undesirable. The final library constituents (shown separately prior to pooling) are shown in the gel image in FIG. 23. The “normal” library smear usually stretches from 175 bp upward. Here, the smallest fragments are >300 bp. Similarly, the largest fragments appear to be 750 bp or larger. Larger fragments do not give rise to optimal libraries. These samples were all twice purified on 80% bead:sample ratios. These samples were pooled into a 16.9 ng/μL pool that, with an estimated average insert size of 400 bp, is about 65 nM. The samples were sequenced.

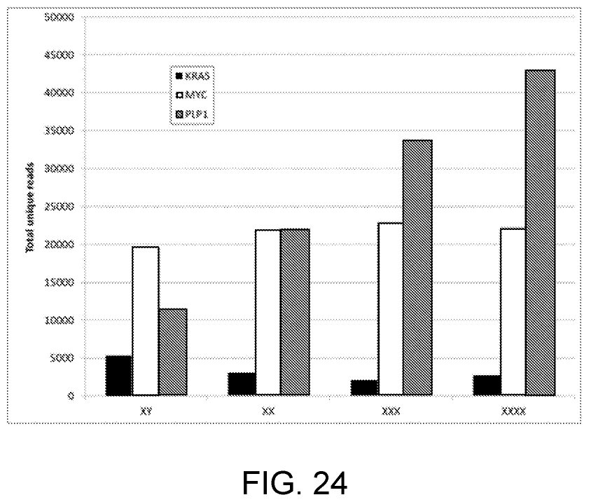

The library-free methods worked well for CNV analyses. Unique read counts for the X-linked gene PLP1 were normalized to the autosomal loci KRAS and MYC and the plot of these data is shown in FIG. 24. The data illustrate that absolute copy number is lost with the library-free procedure (the “copies” of KRAS relative to MYC are no longer comparable). However, relative copy number (the change of PLP1 relative to the autosomal normalizers) is robustly detected. The sequencing results also showed striking features related to read start sites relative to probe.

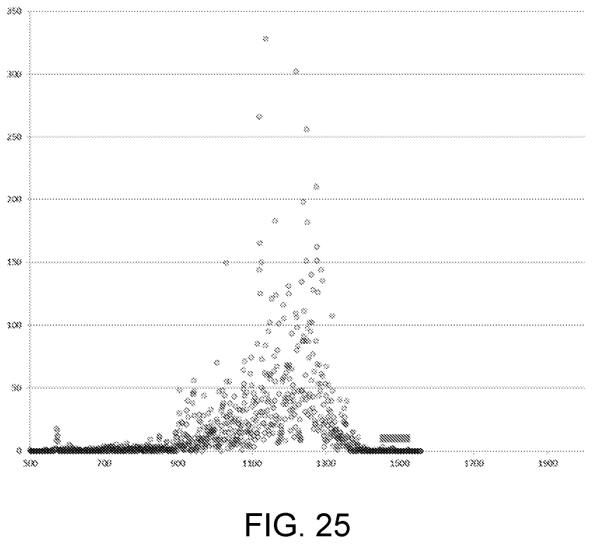

FIG. 25 shows that reads are detected as far as 900 bp from the probe; and between coordinates 1100 and 1300 every single start point is used multiple times. These data indicated that reads start at every single possible base position and that there is little ligation/processing bias. In addition, there are very few reads that start within 100 bp of the probe, consistent with the very large size distribution of the library that was observed on gels.

### Example 4

### Profiling of Genomic DNA

The following example demonstrates the profiling of one microgram of genomic DNA. This genomic DNA can be isolated from whole blood cells, from the buffy coat, from peripheral blood mononuclear cells, or from other samples and tissues as described herein. In reality, all of these are similar sources of nucleated leukocytes that include T cells that have alpha and beta chain TCRs. The steps described in this protocol are illustrated in FIGS. 3-9.

The adaptor for this Example was made from oligos 596 (J-probe-part, CCGCTTAAGTCTACACTAC/3ddC/, SEQ ID NO: 233) and 597 (J-probe-lig, /5Phos/GGTAGTGTAGACTTAAGCGGCTATAGG, SEQ ID NO: 234). 20 μL of each oligo was combined in 160 μL of TEzero+25 mM NaCl to generate a duplex with a final concentration of 10 μM.

The PCR primer for this experiment was oligo 489 (ACC4_27, CCTATAGCCGCTTAAGTCTACACTACC, SEQ ID NO: 228). 50 μL of oligo 489 was combined with 450 μL of TEzero to obtain 10 μM PCR primer.

The following oligonucleotides were also used, as described below: 568 PCR Primer post V-hyb (SEQ ID NO 229); 571 Forward Sequencing Primer (SEQ ID NO: 230); 573 Reverse Sequencing Primer (SEQ ID NO: 231); and 606 Index Sequencing Primer (SEQ ID NO: 235).

In separate reactions, 130 μL of gDNA was sonicated from patient samples VSC7-2, 7-3, 7-4 and 7-5 to 300 bp. 125 μL of sonicated gDNA was added to 150 μL of beads. The mixture was washed twice with 70% EtOH. The pellets were resuspended in 50 μL TEZ. 1000 ng of sonicated gDNA was added to a new tube. Standard end repair was performed (ST1, ST2). Each end repaired sample was captured with: 12.5 μL of 1.0 nM TRAJ Probe+12.5 μL of 1.0 nM TRBJ Probe. The mixture was heated to 98° C. for 2 minutes, and 112.5 μL of hybridization buffer was added. Run on O/N at 65° C. hybridization.

Following hybridization, the mixture was washed as followed. 150 μL of the hybridization reactions was mixed with 40 μL of washed MyOne streptavidin beads in 1 mL TT. The mixture was incubated for 30 minutes with occasional mixing. Beads were pulled out and resuspended in 400 μL TT. Two 200 μL aliquots were separated in PCR strip tubes. The beads were pulled down and resuspended in 200 μL per tube wash buffer, incubated at 45° C. for 5 minutes, pulled out and resuspended in 200 μL TEzero, followed by pulled out and resuspended in 20 μL per tube TEzero.

For T4 extension, 80 μL of T4 mix containing 52.5 μL water, 10 μL 10× CutSmart buffer, 15 μL 50% PEG₈₀₀₀, 1 μL of 10 mM dNTPs, 1 μL T4 Gene 32 protein, and 0.5 μL T4 DNA polymerase was prepared. The mixture was incubated at 20° C. for 15 minutes followed by 70° C. for 10 minutes. The beads were pulled out and resuspended in 200 μL TEzero, pulled out and resuspended in 50 μL TEzero. 20 μL of adaptor was added and 30 μL of standard ligation cocktail (10 μL 10× ligation buffer, 15 μL 50% PEG₈₀₀₀, 5 μL T4 DNA ligation buffer) was added. The standard ligation protocol was run (60 minutes at 20° C., followed by 10 minutes at 65° C.).

The beads were pulled out and resuspended in 20 μL TEzero. 80 μL of “C+P” PCR mix: 50 μL 2× master blend, 10 μL TCR PCR primer 489 (SEQ ID NO: 228), and 20 μL water was added. The sequence was amplified for 5 cycles.

The beads were pulled out, and 60 μL of supernatant was added to 240 μL post C+P PCR mix: 120 μL 2× master blend, 24 μL TCR primer 489 (SEQ ID NO: 228), and 96 μL water. The amplification was monitored by qPCR.

All samples were amplified for 10 cycles (regardless of qPCR results). The beads were purified, and resuspended in 20 μL H₂O for a total of 40 μL H₂0. Each 40 μL sample was captured by adding: 10 μL of 1.0 nM TRAV Probe+10 μL of 1.0 nM TRBV Probe. The mixture was heated to 98° C. for 2 minutes. 90 μL of hybridization buffer was added, and run on O/N 65° C. hybridization.

The mixture was washed post hybridization by combining 150 μL hybridization reactions with 40 μL of washed MyOne streptavidin beads in 1 mL TT. The mixture was incubated for 30 minutes with occasional mixing. The beads were pulled out and resuspended in 400 μL TT. Two 200 μL aliquots were split in PCR strip tubes. The beads were pulled out, resuspended in 200 μL per tube wash buffer, and incubated at 45° C. for 5 minutes. The beads were pulled out and resuspended in 200 μL TEzero, and then pulled out and resuspended in 20 μL per tube TEzero.

80 μL of “C+P” PCR mix was added: 50 μL 2× master blend, 10 μL TCR PCR primer 568 (SEQ ID NO: 229), 10 μL TCR PCR index primer, and 20 μL water. The mixture was amplified for 5 cycles, the beads pulled out, and 60 μL of supernatant was added to 240 μL post C+P PCR mix: 120 μL 2× master blend, 12 μL TCR PCR primer 568 (SEQ ID NO: 229), 12 μL TCR PCR index primer (including index primers 607 (SEQ ID NO: 236), 608 (SEQ ID NO: 237), 623 (SEQ ID NO: 252), and 624 (SEQ ID NO: 253) for patient samples 7-2, 7-3, 7-4 and 7-5, respectively), and 96 μL water. Amplification was monitored by qPCR. Beads were purified by resuspending in 20 μL TEZ for a total of 40 μL TEZ.

Follow standard MiSeq protocol. Use the following primers in the corresponding MiSeq wells. Primer 571 FTCSP (SEQ ID NO: 23) to 18 Primer; 606 ITCSP (SEQ ID NO: 235) to 19 Primer; and 573 RTCSP (SEQ ID NO: 231) to 20 Primer.

The raw output from the Illumina MiSeq run produced approximately 8 million sequencing reads, about 2 million reads per patient sample after parsing the data using the sample index information. The data for each patient was filtered in several steps that included: discarding reads that did not have a legitimate V region or J region probe sequence; discarding reads that did not have a protein coding open reading frame in the CDR3 region between the V and the J probes (Importantly, the observed distribution of CDR3 sequence lengths (average=36 bases for alpha chains and 39 bases for beta chains) was concordant with previous literature reports); identifying redundant reads into a single, consensus TCR “unique sequence”; classifying unique read sets into alpha or beta chains; classifying alpha unique reads or beta unique reads according to their V and J regions; counting the number of TCRs in each V/J intersection (pixel); and presenting the population distribution of TCRs in patient series 7-2 through 7-5 in heat maps.

Approximately 5000 unique alpha and 5000 unique beta TCR sequences were observed in each sample (the range was 3217 to 7684 unique sequences). An example of a heat map for one alpha chain sample is shown in FIG. 10.

One microgram of human genomic DNA is the equivalent of about 150,000 diploid genomes, or, in other words, representative of 150,000 cells. In whole blood, roughly 4-7% of nucleated cells are T cells. Therefore, the expectation is that 6000 to 10,500 unique TCRs in each sample should be observed. The observed density of about 5000 unique TCRs is consistent with this expectation, especially when the fact that cancer patients are often immunosuppressed by therapy is taken into account. The TCR repertoire produced by the methods provided herein is likely to reflect a snapshot of the peripheral, circulating T cells present in a sample. Modifying J probe tags will expand the detection of redundant clones and on profiling of the tumor infiltrating T cells in resected tumor tissue.

Development of the method requires several iterations that were not initially obvious from a priori consideration of the assay. The method has significant clinical utility in applications such as infectious disease monitoring and assessment of the efficacy of immune-oncology therapies.

It is to be understood that the description, specific examples and data, while indicating exemplary embodiments, are given by way of illustration and are not intended to limit the various embodiments of the present disclosure. Various changes and modifications within the present disclosure will become apparent to the skilled artisan from the description and data contained herein, and thus are considered part of the various embodiments of this disclosure.

What is claimed is:

1. A method of identifying a rearranged adaptive immune response gene comprising:
a. obtaining a sample comprising genomic DNA;
b. isolating genomic DNA from the sample;
c. capturing a rearranged adaptive immune response gene from the isolated genomic DNA by sequential hybridization, wherein the sequential hybridization comprises:
i. hybridizing the genomic DNA with a first set of probes specific to a first portion of the rearranged adaptive immune response gene to generate a hybridized sequence;
ii. extending the first set of probes to generate a first extended sequence;
iii. purifying or isolating the first extended sequence;
iv. hybridizing the purified first extended sequence with a second set of probes specific to a second portion of the rearranged adaptive immune response gene;
v. extending the second set of probes to generate a second extended sequence;
d. amplifying the second extended sequence; and
e. sequencing the second extended sequence.

2. The method of claim 1, further comprising fragmenting and end-repairing the genomic DNA prior to sequential hybridization.

3. The method of any one of claims 1-2, wherein the sample is obtained from a tissue or a biofluid.

4. The method of any one of claims 1-3, wherein the sample is obtained from a tumor tissue, a region proximal to a tumor tissue, an organ tissue, peripheral tissue, lymph, urine, cerebral spinal fluid, a buffy coat isolate, whole blood, peripheral blood, bone marrow, amniotic fluid, breast milk, plasma, serum, aqueous humor, vitreous humor, cochlear fluid, saliva, stool, sweat, vaginal secretions, semen, bile, tears, mucus, sputum, or vomit.

5. The method of any one of claims 1-4, wherein the sample comprises adaptive immune cells.

6. The method of any one of claims 1-5, wherein the sample comprises one or more immune cells, such as T cells.

7. The method of any one of claims 1-6, wherein the rearranged adaptive immune response gene is encoded by the T cell receptor (TCR) alpha gene (TRA), the TCR beta gene (TRB), the TCR delta gene (TRD), the TCR gamma gene (TRG), the antibody heavy chain gene (IGH), the kappa light chain antibody gene (IGK), and/or the lambda light chain antibody gene (IGL).

8. The method of any one of claims 1-7, the first portion of the rearranged adaptive immune response gene is a CDR3-encoding region, comprising a V, D, or J region of the rearranged adaptive immune response gene.

9. The method of any one of claims 1-8, wherein the first extended sequence is copied with T4 DNA polymerase and T4 gene 32 protein.

10. The method of claim 9, wherein extending is performed in a solution containing polyethylene glycol (PEG).

11. The method of claim 10, wherein the PEG has an average molecular weight of 8000 daltons (PEG₈₀₀₀).

12. The method of any one of claims 10-11, wherein PEG is present in an amount of about 7.5% w/v.

13. The method of any one of claims 1-12, further comprising ligating an amplification adaptor to the first extended sequence.

14. The method of any one of claims 1-13, wherein amplifying is performed by polymerase chain reaction (PCR).

15. The method of any one of claims 1-14, wherein the first set of probes comprises J region sequences of human TCR alpha (TRA), human TCR beta (TRB), human TCR gamma (TRG), human TCR delta (TRG), a human antibody heavy chain (IGH), a human kappa light chain antibody (IGK), or a human lambda light chain antibody (IGL).

16. The method of any one of claims 1-15, wherein the first set of probes comprises V region sequences of human TRA, human TRB, human TRG, human TRD, human IGH, human IGK, and/or human IGL.

17. The method of any one of claims 1-16, wherein the second set of probes comprises J region sequences of human TRA, human TRB, human TRG, human TRD, human IGH, human IGK, and/or human IGL.

18. The method of any one of claims 1-17, wherein the second set of probes comprises V region sequences of human TRA, human TRB, human TRG, human TRD, human IGH, human IGK, and/or human IGL.

19. The method of any one of claims 1-18, wherein the first set of probes comprises a DNA sequence tag for identification of specific clones.

20. The method of claim 19, wherein the DNA sequence tag comprises a nucleic acid sequence of NN, NNN, NNNN, NNNNN, NNNNNN, NNNNNNN, NNNNNNNN, NNNNNNNNN, or NNNNNNNNNN, wherein N is A, T, G, or C.

21. The method of any one of claims 19-20, wherein the DNA sequence tags, the first and second set of probes, and the captured sequences are all used in informatic identification of clones.

22. The method of any one of claims 1-23, wherein the sample comprises a plurality of rearranged genomic sequences.

23. The method of any one of claims 1-24, further comprising determining the frequency of specific T cell clones, B cell clones, or both in the sample to determine a T cell immune repertoire, a B cell repertoire, or both in the sample.

24. The method of claim 1, further comprising profiling circulating nucleic acids, TCR repertoire, or Ab repertoire in a whole blood sample.

25. The method of claim 24, wherein profiling comprises a determination of the characteristics of a population of nucleic acids, TCR repertoire, or Ab repertoire in a sample.

26. The method of claim 1, further comprising assessing both circulating nucleic acid and immune repertoire from a single whole blood sample.

27. The method of claim 1, wherein an amount of single cell genomic DNA is increased by whole genome amplification prior to analysis.

28. The method of claim 1, wherein single cell analysis is used to identify pairing between alpha and beta chain TCR within a single cell.

29. The method of any one of claims 1-28, wherein the first set of probes comprises a nucleic acid having at least 90% sequence identity to one or more sequences as defined in any one of SEQ ID NOs: 62-128.

30. The method of any one of claims 1-29, wherein the second set of probes comprises a nucleic acid having at least 90% sequence identity to one or more sequences as defined in any one of SEQ ID NO: 129-227.
